SEQUENCE LISTING (52 sequences)

SEQ ID NO: 1 - DNA, 3921 bp, Homo Sapien
cggaagttgcgcgcaggccggcgggcgggagcggacaccgaggccggcgtgcaggcgtgcgggtgtgcgggagccgggctcggggggatcggaccgagagcgagaagcgcggcatggagctccaggcagcccgcgcctgcttcgccctgctgtggggctgtgcgctggccgcggccgcggcggcgcagggcaaggaagtggtactgctggactttgctgcagctggaggggagctcggctggctcacacacccgtatggcaaagggtgggacctgatgcagaacatcatgaatgacatgccgatctacatgtactccgtgtgcaacgtgatgtctggcgaccaggacaactggctccgcaccaactgggtgtaccgaggagaggctgagcgtaacaactttgagctcaactttactgtacgtgactgcaacagcttccctggtggcgccagctcctgcaaggagactttcaacctctactatgccgagtcggacctggactacggcaccaacttccagaagcgcctgttcaccaagattgacaccattgcgcccgatgagatcaccgtcagcagcgacttcgaggcacgccacgtgaagctgaacgtggaggagcgctccgtggggccgctcacccgcaaaggcttctacctggccttccaggatatcggtgcctgtgtggcgctgctctccgtccgtgtctactacaagaagtgccccgagctgctgcagggcctggcccacttccctgagaccatcgccggctctgatgcaccttccctggccactgtggccggcacctgtgtggaccatgccgtggtgccaccggggggtgaagagccccgtatgcactgtgcagtggatggcgagtggctggtgcccattgggcagtgcctgtgccaggcaggctacgagaaggtggaggatgcctgccaggcctgctcgcctggattttttaagtttgaggcatctgagagcccctgcttggagtgccctgagcacacgctgccatcccctgagggtgccacctcctgcgagtgtgaggaaggcttcttccgggcacctcaggacccagcgtcgatgccttgcacacgacccccttccgccccacactacctcacagccgtgggcatgggtgccaaggtggagctgcgctggacgccccctcaggacagcgggggccgcgaggacattgtctacagcgtcacctgcgaacagtgctggcccgagtctggggaatgcgggccgtgtgaggccagtgtgcgctactcggagcctcctcacggactgacccgcaccagtgtgacagtgagcgacctggagccccacatgaactacaccttcaccgtggaggcccgcaatggcgtctcaggcctggtaaccagccgcagcttccgtactgccagtgtcagcatcaaccagacagagccccccaaggtgaggctggagggccgcagcaccacctcgcttagcgtctcctggagcatccccccgccgcagcagagccgagtgtggaagtacgaggtcacttaccgcaagaagggagactccaacagctacaatgtgcgccgcaccgagggtttctccgtgaccctggacgacctggccccagacaccacctacctggtccaggtgcaggcactgacgcaggagggccagggggccggcagcaaggtgcacgaattccagacgctgtccccggagggatctggcaacttggcggtgattggcggcgtggctgtcggtgtggtcctgcttctggtgctggcaggagttggcttctttatccaccgcaggaggaagaaccagcgtgcccgccagtccccggaggacgtttacttctccaagtcagaacaactgaagcccctgaagacatacgtggacccccacacatatgaggaccccaaccaggctgtgttgaagttcactaccgagatccatccatcctgtgtcactcggcagaaggtgatcggagcaggagagtttggggaggtgtacaagggcatgctgaagacatcctcggggaagaaggaggtgccggtggccatcaagacgctgaaagccggctacacagagaagcagcgagtggacttcctcggcgaggccggcatcatgggccagttcagccaccacaacatcatccgcctagagggcgtcatctccaaatacaagcccatgatgatcatcactgagtacatggagaatggggccctggacaagttccttcgggagaaggatggcgagttcagcgtgctgcagctggtgggcatgctgcggggcatcgcagctggcatgaagtacctggccaacatgaactatgtgcaccgtgacctggctgcccgcaacatcctcgtcaacagcaacctggtctgcaaggtgtctgactttggcctgtcccgcgtgctggaggacgaccccgaggccacctacaccaccagtggcggcaagatccccatccgctggaccgccccggaggccatttcctaccggaagttcacctctgccagcgacgtgtggagctttggcattgtcatgtgggaggtgatgacctatggcgagcggccctactgggagttgtccaaccacgaggtgatgaaagccatcaatgatggcttccggctccccacacccatggactgcccctccgccatctaccagctcatgatgcagtgctggcagcaggagcgtgcccgccgccccaagttcgctgacatcgtcagcatcctggacaagctcattcgtgcccctgactccctcaagaccctggctgactttgacccccgcgtgtctatccggctccccagcacgagcggctcggagggggtgcccttccgcacggtgtccgagtggctggagtccatcaagatgcagcagtatacggagcacttcatggcggccggctacactgccatcgagaaggtggtgcagatgaccaacgacgacatcaagaggattggggtgcggctgcccggccaccagaagcgcatcgcctacagcctgctgggactcaaggaccaggtgaacactgtggggatccccatctgagcctcgacagggcctggagccccatcggccaagaatacttgaagaaacagagtggcctccctgctgtgccatgctgggccactggggactttatttatttctagttctttcctccccctgcaacttccgctgaggggtctcggatgacaccctggcctgaactgaggagatgaccagggatgctgggctgggccctctttccctgcgagacgcacacagctgagcacttagcaggcaccgccacgtcccagcatccctggagcaggagccccgccacagccttcggacagacatataggatattcccaagccgaccttccctccgccttctcccacatgaggccatctcaggagatggagggcttggcccagcgccaagtaaacagggtacctcaagccccatttcctcacactaagagggcagactgtgaacttgactgggtgagacccaaagcggtccctgtccctctagtgccttctttagaccctcgggccccatcctcatccctgactggccaaacccttgctttcctgggcctttgcaagatgcttggttgtgttgaggtttttaaatatatattttgtactttgtggagagaatgtgtgtgtgtggcagggggccccgccagggctggggacagagggtgtcaaacattcgtgagctggggactcagggaccggtgctgcaggagtgtcctgcccatgccccagtcggccccatctctcatccttttggataagtttctattctgtcagtgttaaagattttgttttgttggacatttttttcgaatcttaatttattattttttttatatttattgttagaaaatgacttatttctgctctggaataaagttgcagatgattcaaaccg

SEQ ID NO: 2 - protein, 976 aa, Homo Sapien
MELQAARACFALLWGCALAAAAAAQGKEVVLLDFAAAGGELGWLTHPYGKGWDLMQNIMNDMPIYMYSVCNVMSGDQDNWLRTNWVYRGEAERNNFELNFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 3 - DNA, 4366 bp, Homo Sapien
  misc_feature (1)...(4366): n = A,T,C or G
tgcattctttgccccaaaactctttcctttggttgtgctaagaggtgatgcccaaggtgcaccacctttcaagaactggatcatgaacaactttatcctcctggaagaacagctcatcaagaaatcccaacaaaagagaagaacttctccctcgaactttaaagtccgcttcttcgtgttaaccaaagccagcctggcatactttgaagatcgtcatgggaagaagcgcacgctgaaggggtccattgagctctcccgaatcaaatgtgttgagattgtgaaaagtgacatcagcatcccatgccactataaatacccgtttcaggtggtgcatgacaactacctcctatatgtgtttgctccagatcgtgagagccggcagcgctgggtgctggcccttaaagaagaaacgaggaataataacagtttggtgcctaaatatcatcctaatttctggatggatgggaagtggaggtgctgttctcagctggagaagcttgcaacaggctgtgcccaatatgatccaaccaagaatgcttcaaagaagcctcttcctcctactcctgaagacaacaggcgaccactttgggaacctgaagaaactgtggtcattgccttatatgactaccaaaccaatgatcctcaggaactcgcactgcggcgcaacgaagagtactgcctgctggacagttctgagattcactggtggagagtccaggacaggaatgggcatgaaggatatgtaccaagcagttatctggtggaaaaatctccaaataatctggaaacctatgagtggtacaataagagtatcagccgagacaaagctgaaaaacttcttttggacacaggcaaagaaggagccttcatggtaagggattccaggactgcaggaacatacaccgtgtctgttttcaccaaggctgttgtaagtgagaacaatccctgtataaagcattatcacatcaaggaaacaaatgacaatcctaagcgatactatgtggctgaaaagtatgtgttcgattccatccctcttctcatcaactatcaccaacataatggaggaggcctggtgactcgactccggtatccagtttgttttgggaggcagaaagccccagttacagcagggctgagatacgggaaatgggtgatcgacccctcagagctcacttttgtgcaagagattggcagtgggcaatttgggttggtgcatctgggctactggctcaacaaggacaaggtggctatcaaaaccattcgggaaggggctatgtcagaagaggacttcatagaggaggctgaagtaatgatgaaactctctcatcccaaactggtgcagctgtatggggtgtgcctggagcaggcccccatctgcctggtgtttgagttcatggagcacggctgcctgtcagattatctacgcacccagcggggactttttgctgcagagaccctgctgggcatgtgtctggatgtgtgtgagggcatggcctacctggaagaggcatgtgtcatccacagagacttggctgccagaaattgtttggtgggagaaaaccaagtcatcaaggtgtctgactttgggatgacaaggttcgttctggatgatcagtacaccagttccacaggcaccaaattcccggtgaagtgggcatccccagaggttttctctttcagtcgctatagcagcaagtccgatgtgtggtcatttggtgtgctgatgtgggaagttttcagtgaaggcaaaatcccgtatgaaaaccgaagcaactcagaggtggtggaagacatcagtaccggatttcggttgtacaagccccggctggcctccacacacgtctaccagattatgaatcactgctggaaagagagaccagaagatcggccagccttctccagactgctgcgtcaactggctgaaattgcagaatcaggactttagtagagactgagtaccaggccacgggctgcagatcctgaatggaggaaggatatgtcctcattccatagagcattagaagctgccaccagcccaggaccctccagaggcagcctggcctgtggcatcagtccctgagtcaccatggaagcagcatcctgaccacagctggcagtcaagccacagctggagggtcagccaccaagctgggagctgagccagaacaggagtgatgtctctgcccttcctctagcctcttgtcacatgtggtgcacaaacctcaacctgacagctttcagacagcattcttgcacttcttagcaacagagagagacatgagtaagacccagattgctatttttattgttatttttaacatgaatctaaagnttatggttccagggactttttatttgacccaacaacacagtatcccaggatatggaggcaaggggaacaaagagcatgagtctttttccaagaaaactggtgagttaagtaagattagagtgagtgtgctctgttgctgtgatgctgtcagccacagcttcctgccgtagagaatgatagagcagctgctcacacaggaggccggatattctgagaagcagctttatgaggttttacagagtatgctgctacctctctccttgaagggagcatggcgagacccattggatggattggggtgaacagttcaggtcccatgcttggagcattgggtatctgatgtctgcaccagaacaagagaacctctgacggtggagaaccatgtggtgcaagaagagatcttaggtctcttcttttataccaagctcatcttttataccaagctgtgcaggtgactatgcctcctcttctgcacagaatgcttccaccagcatcctgagaagaaatgattacttctgaaaaacatccttttttccagcctctgggaatcagccccccctctctgcactatccgatcctcatcaacagagggcagcattgtgttggtcaatgttcccttggcgagcaattgaaacttgtttaggccctagggttgagcaattttaaggttgagactccaagtctcctaaaattctaggagagaaataaagagtctgtttttgctcaaaccatcaggatggaaacagtcaggcactgactggggtgcttccaagaggcatgagagtgcctactctggcttgagcacttctatatgcaaggtgaatatgtactgagctaggagacttccctgcaaaatctctgttcaccctgggttcacatccccatgaggtaatattattattcccattttacaaataatgtaactgaggctttaaaaagccaagacatctgcccaaagtgatggaactagaaagtctagagctggtattctagcccaaatctgtctgaccgcaatacacagattctttattcctattcgacactggcttctactgaaaatgaaacggattgcagagggaataaatacaaagatggaaagccagtaaagaagtcagtatagaaccactagcgaatagtgttgctctggcacagaccactgtggttgatggcatggccctccaacttggaataggattttccttttcctattctgtatccttaccttggtcatgttaatgactttggagttattcagttaatgaccctttaattctcacaaccaaccagtcatgttgcttgaagccatttatagacgagcttcaaagcaactttaaaagattcttctgtagaagtatgagttcttcctttaattatcattccaactttcagctgtagtcttcttgaacacttcatgaggagggacattccctgatataagagaggatggtgttgcaattggctctttctaaatcatgtgacgttttgactggcttgagattcagatgcataatttttaattataattattgtgaagtggagagcctcaagataaaactctgtcattcagaagatgattttactcagcttatccaaaattatctctgtttactttttagaattttgtacattatcttttgggatccttaattagagatgatttctggaacattcagtctagaaagaaaacattggaattgactgatctctgtggtttggtttagaaaattcccctgtgcatggtattacctttttcaagctcagattcatctaatcctcaactgtacatgtgtacattcttcacctcctggtgccctatcccgcaaaatgggcttcctgcctggtttttctcttctcacattttttaaatggtcccctgtgtttgtagagaactcccttatacagagttttggttctagttttatttcgtagattttgcattttgtaccttttgagactatgtatttatatttggatcagatgcatatttattaatgtacagtcactgctagtgttcaaaataaaaatgttacaaat

SEQ ID NO: 4 - protein, 620 aa, Homo Sapien
MNNFILLEEQLIKKSQQKRRTSPSNFKVRFFVLTKASLAYFEDRHGKKRTLKGSIELSRIKCVEIVKSDISIPCHYKYPFQVVHDNYLLYVFAPDRESRQRWVLALKEETRNNNSLVPKYHPNFWMDGKWRCCSQLEKLATGCAQYDPTKNASKKPLPPTPEDNRRPLWEPEETVVIALYDYQTNDPQELALRRNEEYCLLDSSEIHWWRVQDRNGHEGYVPSSYLVEKSPNNLETYEWYNKSISRDKAEKLLLDTGKEGAFMVRDSRTAGTYTVSVFTKAVVSENNPCIKHYHIKETNDNPKRYYVAEKYVFDSIPLLINYHQHNGGGLVTRLRYPVCFGRQKAPVTAGLRYGKWVIDPSELTFVQEIGSGQFGLVHLGYWLNKDKVAIKTIREGAMSEEDFIEEAEVMMKLSHPKLVQLYGVCLEQAPICLVFEFMEHGCLSDYLRTQRGLFAAETLLGMCLDVCEGMAYLEEACVIHRDLAARNCLVGENQVIKVSDFGMTRFVLDDQYTSSTGTKFPVKWASPEVFSFSRYSSKSDVWSFGVLMWEVFSEGKIPYENRSNSEVVEDISTGFRLYKPRLASTHVYQIMNHCWKERPEDRPAFSRLLRQLAEIAESGL

SEQ ID NO: 5 - DNA, 3967 bp, Homo Sapien
ctgcaggaattccgatccttccgcaggttcacctacggaaaccttgttacgacttttacttcctctagatagtcaagttcgaccgtcttctcagcgctccgccagggccgtgggccgaccccggcggggccgatccgagggcctcactaaaccatccaatcggtagtagcgacgggcggtgtgtacaaagggcagggacttaatcaacgcaagcttatgacccgcacttactgggaattcctcgttcatggggaataattgcaatccccgatccccatcacgaatggggttcaacgggttacccgcgcctgccggcgtagggtaggcacacgctgagccagtcagtgtagcgcgcgtgcagccccggacatctaagggcatcacagacctgttattgctcaatctcgggtggctgaacgccacttgtccctctaagaagttgggggacgccgaccgctcgggggtcgcgtaactagttagcatgccagagtctcgttcgttatcggaattaaccagacaaatcgctccaccaactaagaacggccatgcaccaccacccacggaatcgagaaagagctatcaatctgtcaatcctgtccgtgtccgggccgggtgaggtttcccgtgttgagtcaaattaagccgcaggctccactcctggtggtgcccttccgtcaattcctttaagtttcagctttgcaaccatactccccccggaacccaaagactttggtttcccggaagctgcccggcgggtcatgggaataacgccgccgcatcgccggtcggcatcgtttatggtcggaactacgacggtatctgatcgtcttcgaacctccgactttcgttcttgattaatgaaaacattcttggcaaatgctttcgctctggtccgtcttgcgccggtccaagaatttcggaattccgcagcggcggccagcagggcggaggctgaggcagcaagctcgctagagagggagaagcagtcgggcgcaggcgcctcctccgcagcccgctccatggtcggcgcccacagcccgcggcggcctgtcttgcgctccacttccttcacatcctcctccgcctcctcgttttcaggcgccgccggcggcgctgtgtggaggcccgcgagctgaaattcgcggtgcgacgggagggagtggagaaggaggtgagggggcccaggatcgcggggcgccctgaggcaaggggacgccggcgggccgaagcgcagcccgccgcccgcaggctcggctccgccactgccgccctcccggtctcctcgcctcggccgccgaggcagggagagaatgagccccgggacccgccgggggacggcccgggccaggcccgggatctagacggccgtagggggaagggagccgccctccccacggcgccttttcggaactgccgtggactcgaggacgctggtcgccggcctcctagggctgtgctgttttgttttgaccctcgcattgtgcagaattaaagtgcagtaaaatgtccactaggaccccattgccaacggtgaatgaacgagacactgaaaaccacacgtcacatggagatgggcgtcaagaagttacctctcgtaccagccgctcaggagctcggtgtagaaactctatagcctcctgtgcagatgaacaacctcacatcggaaactacagactgttgaaaacaatcggcaaggggaattttgcaaaagtaaaattggcaagacatatccttacaggcagagaggttgcaataaaaataattgacaaaactcagttgaatccaacaagtctacaaaagctcttcagagaagtaagaataatgaagattttaaatcatcccaatatagtgaagttattcgaagtcattgaaactgaaaaaacactctacctaatcatggaatatgcaagtggaggtgaagtatttgactatttggttgcacatggcaagatgaaggaaaaagaagcaagatctaaatttagacagggttgtcaagctggacagactattaaagttcaagtctcctttgatttgcttagtctgatgtttacatttattgtgtctgcagttcaatactgccatcagaaacggatcgtacatcgagacctcaaggctgaaaatctattgttagatgccgatatgaacattaaaatagcagatttcggttttagcaatgaatttactgttggcggtaaactcgacacgttttgtggcagtcctccatacgcagcacctgagctcttccagggcaagaaatatgacgggccagaagtggatgtgtggagtctgggggtcattttatacacactagtcagtggctcacttccctttgatgggcaaaacctaaaggaactgagagagagagtattaagagggaaatacagaattcccttctacatgtctacagactgtgaaaaccttctcaaacgtttcctggtgctaaatccaattaaacgcggcactctagagcaaatcatgaaggacaggtggatcaatgcagggcatgaagaagatgaactcaaaccatttgttgaaccagagctagacatctcagaccaaaaaagaatagatattatggtgggaatgggatattcacaagaagaaattcaagaatctcttagtaagatgaaatacgatgaaatcacagctacatatttgttattggggagaaaatcttcagagctggatgctagtgattccagttctagcagcaatctttcacttgctaaggttaggccgagcagtgatctcaacaacagtactggccagtctcctcaccacaaagtgcagagaagtgtttcttcaagccaaaagcaaagacgctacagtgaccatgctggaccagctattccttctgttgtggcgtatccgaaaaggagtcagaccagcactgcagatagtgacctcaaagaagatggaatttcctcccggaaatcaagtggcagtgctgttggaggaaagggaattgctccagccagtcccatgcttgggaatgcaagtaatcctaataaggcggatattcctgaacgcaagaaaagctccactgtccctagtagtaacacagcatctggtggaatgacacgacgaaatacttatgtttgcagtgagagaactacagctgatagacactcagtgattcagaatggcaaagaaaacagcactattcctgatcagagaactccagttgcttcaacacacagtatcagtagtgcagccaccccagatcgaatccgcttcccaagaggcactgccagtcgtagcactttccacggccagccccgggaacggcgaaccgcaacatataatggccctcctgcctctcccagcctgtcccatgaagccacaccattgtcccagactcgaagccgaggctccactaatctctttagtaaattaacttcaaaactcacaaggagaaacatgtcattcaggtttatcaaaaggcttccaactgaatatgagaggaacgggagatatgagggctcaagtcgcaatgtatctgctgagcaaaaagatgaaaacaaagaagcaaagcctcgatccctacgcttcacctggagcatgaaaaccactagttcaatggatcccggggacatgatgcgggaaatccgcaaagtgttggacgccaataactgcgactatgagcagagggagcgcttcttgctcttctgcgtccacggagatgggcacgcggagaacctcgtgcagtgggaaatggaagtgtgcaagctgccaagactgtctctgaacggggtccggtttaagcggatatcggggacatccatagccttcaaaaatattgcttccaaaattgccaatgagctaaagctgtaacccagtgattatgatgtaaattaagtagcaagtaaagtgttttcctgaacactgatggaaatgtatagaataatatttaggcaataacgtctgcatcttctaaatcatgaaattaaagtctgaggacgagagc

SEQ ID NO: 6 - protein, 776 aa, Homo Sapien
MSTRTPLPTVNERDTENHTSHGDGRQEVTSRTSRSGARCRNSIASCADEQPHIGNYRLLKTIGKGNFAKVKLARHILTGREVAIKIIDKTQLNPTSLQKLFREVRIMKILNHPNIVKLFEVIETEKTLYLIMEYASGGEVFDYLVAHGKMKEKEARSKFRQGCQAGQTIKVQVSFDLLSLMFTFIVSAVQYCHQKRIVHRDLKAENLLLDADMNIKIADFGFSNEFTVGGKLDTFCGSPPYAAPELFQGKKYDGPEVDVWSLGVILYTLVSGSLPFDGQNLKELRERVLRGKYRIPFYMSTDCENLLKRFLVLNPIKRGTLEQIMKDRWINAGHEEDELKPFVEPELDISDQKRIDIMVGMGYSQEEIQESLSKMKYDEITATYLLLGRKSSELDASDSSSSSNLSLAKVRPSSDLNNSTGQSPHHKVQRSVSSSQKQRRYSDHAGPAIPSVVAYPKRSQTSTADSDLKEDGISSRKSSGSAVGGKGIAPASPMLGNASNPNKADIPERKKSSTVPSSNTASGGMTRRNTYVCSERTTADRHSVIQNGKENSTIPDQRTPVASTHSISSAATPDRIRFPRGTASRSTFHGQPRERRTATYNGPPASPSLSHEATPLSQTRSRGSTNLFSKLTSKLTRRNMSFRFIKRLPTEYERNGRYEGSSRNVSAEQKDENKEAKPRSLRFTWSMKTTSSMDPGDMMREIRKVLDANNCDYEQRERFLLFCVHGDGHAENLVQWEMEVCKLPRLSLNGVRFKRISGTSIAFKNIASKIANELKL

SEQ ID NO: 7 - DNA, 1559 bp, Homo Sapien
caggatgtaaatgagcacactgctggcccatgcgcctcggggctgtagagggcagcctcagaggcactgggcattcctggcaccatggatgacgctgctgtcctcaagcgacgaggctacctcctggggataaatttaggagagggctcctatgcaaaagtaaaatctgcttactctgagcgcctgaagttcaatgtggcgatcaagatcatcgaccgcaagaaggcccccgcagacttcttggagaaattccttccccgggaaattgagattctggccatgttaaaccactgctccatcattaagacctacgagatctttgagacatcacatggcaaggtctacatcgtcatggagctcgcggtccagggcgacctcctcgagttaatcaaaacccggggagccctgcatgaggacgaagctcgcaagaagttccaccagctttccttggccatcaagtactgccacgacctggacgtcgtccaccgggacctcaagtgtgacaaccttctccttgacaaggacttcaacatcaagctgtccgacttcagcttctccaagcgctgcctgcgggatgacagtggtcgaatggccttaagcaagaccttctgtgggtcaccagcgtatgcggccccagaggtgctgcagggcattccctaccagcccaaggtgtacgacatctggagcctaggcgtgatcctctacatcatggtctgcggctccatgccctacgacgactccaacatcaagaagatgctgcgtatccagaaggagcaccgcgtcaacttcccacgctccaagcacctgacaggcgagtgcaaggacctcatctaccacatgctgcagcccgacgtcaaccggcggctccacatcgacgagatcctcagccactgctggatgcagcccaaggcacggggatctccctctgtggccatcaacaaggagggggagagttcccggggaactgaacccttgtggacccccgaacctggctctgacaagaagtctgccaccaagctggagcctgagggagaggcacagccccaggcacagcctgagacaaaacccgaggggacagcaatgcaaatgtccaggcagtcggagatcctgggtttccccagcaagccgtcgactatggagacagaggaagggcccccccaacagcctccagagacgcgggcccagtgagcttcttgcggcccagggaatgagatggagctcacggcttaaagcccaagctctgaagaagtcaagggtggagccagagaaggaaggcagtcccagatgagcctctattttcatcagcttcttctctctccccttgaacttggtaacccacatggttctcccgtggcccctaggtggatgaggccaaagtcaaatccaaggctgagacagtcgtgcgactcctactcccccagagcgtgacccggagcaggtgctggacacagagcctgtctcagcagagggtccccactggccgcaacggctcagtgacagcaagagcaggaagagcagcaggaaggcaccgctgtccaccttgggcaccatttatcctc

SEQ ID NO: 8 - protein, 367 aa, Homo Sapien
MDDAAVLKRRGYLLGINLGEGSYAKVKSAYSERLKFNVAIKIIDRKKAPADFLEKFLPREIEILAMLNHCSIIKTYEIFETSHGKVYIVMELAVQGDLLELIKTRGALHEDEARKKFHQLSLAIKYCHDLDVVHRDLKCDNLLLDKDFNIKLSDFSFSKRCLRDDSGRMALSKTFCGSPAYAAPEVLQGIPYQPKVYDIWSLGVILYIMVCGSMPYDDSNIKKMLRIQKEHRVNFPRSKHLTGECKDLIYHMLQPDVNRRLHIDEILSHCWMQPKARGSPSVAINKEGESSRGTEPLWTPEPGSDKKSATKLEPEGEAQPQAQPETKPEGTAMQMSRQSEILGFPSKPSTMETEEGPPQQPPETRAQ

SEQ ID NO: 9 - DNA, 3151 bp, Homo Sapien
ttttccttgagcacagcactttgtgacctttgatgtaaacatcaaacacagccccctttcctgtcttcgcatccaggaaataggttagtttcagacaagcctgcttgccggagctcagcagacaccaggccttccgggcaggcctggcccaccgtgggcctcagagctgctgctggggcattcagaaccggctctccattggcattgggaccagagaccccgcaagtggcctgtttgcctggacatccacctgtacgtccccaggtttcgggaggcccaggggcgatgccagaccccgcggcgcacctgcccttcttctacggcagcatctcgcgtgccgaggccgaggagcacctgaagctggcgggcatggcggacgggctcttcctgctgcgccagtgcctgcgctcgctgggcggctatgtgctgtcgctcgtgcacgatgtgcgcttccaccactttcccatcgagcgccagctcaacggcacctacgccattgccggcggcaaagcgcactgtggaccggcagagctctgcgagttctactcgcgcgaccccgacgggctgccctgcaacctgcgcaagccgtgcaaccggccgtcgggcctcgagccgcagccgggggtcttcgactgcctgcgagacgccatggtgcgtgactacgtgcgccagacgtggaagctggagggcgaggccctggagcaggccatcatcagccaggccccgcaggtggagaagctcattgctacgacggcccacgagcggatgccctggtaccacagcagcctgacgcgtgaggaggccgagcgcaaactttactctggggcgcagaccgacggcaagttcctgctgaggccgcggaaggagcagggcacatacgccctgtccctcatctatgggaagacggtgtaccactacctcatcagccaagacaaggcgggcaagtactgcattcccgagggcaccaagtttgacacgctctggcagctggtggagtatctgaagctgaaggcggacgggctcatctactgcctgaaggaggcctgccccaacagcagtgccagcaacgcctcaggggctgctgctcccacactcccagcccacccatccacgttgactcatcctcagagacgaatcgacaccctcaactcagatggatacacccctgagccagcacgcataacgtccccagacaaaccgcggccgatgcccatggacacgagcgtgtatgagagcccctacagcgacccagaggagctcaaggacaagaagctcttcctgaagcgcgataacctcctcatagctgacattgaacttggctgcggcaactttggctcagtgcgccagggcgtgtaccgcatgcgcaagaagcagatcgacgtggccatcaaggtgctgaagcagggcacggagaaggcagacacggaagagatgatgcgcgaggcgcagatcatgcaccagctggacaacccctacatcgtgcggctcattggcgtctgccaggccgaggccctcatgctggtcatggagatggctgggggcgggccgctgcacaagttcctggtcggcaagagggaggagatccctgtgagcaatgtggccgagctgctgcaccaggtgtccatggggatgaagtacctggaggagaagaactttgtgcaccgtgacctggcggcccgcaacgtcctgctggttaaccggcactacgccaagatcagcgactttggcctctccaaagcactgggtgccgacgacagctactacactgcccgctcagcagggaagtggccgctcaagtggtacgcacccgaatgcatcaacttccgcaagttctccagccgcagcgatgtctggagctatggggtcaccatgtgggaggccttgtcctacggccagaagccctacaagaagatgaaagggccggaggtcatggccttcatcgagcagggcaagcggatggagtgcccaccagagtgtccacccgaactgtacgcactcatgagtgactgctggatctacaagtgggaggatcgccccgacttcctgaccgtggagcagcgcatgcgagcctgttactacagcctggccagcaaggtggaagggcccccaggcagcacacagaaggctgaggctgcctgtgcctgagctcccgctgcccaggggagccctccacgccggctcttccccaccctcagccccaccccaggtcctgcagtctggctgagccctgcttggttgtctccacacacagctgggctgtggtagggggtgtctcaggccacaccggccttgcattgcctgcctggccccctgtcctctctggctggggagcagggaggtccgggagggtgcggctgtgcagcctgtcctgggctggtggctcccggagggccctgagctgagggcattgcttacacggatgccttcccctgggccctgacattggagcctgggcatcctcaggtggtcaggcgtagatcaccagaataaacccagcttccctcttgtctgagcgccctcatcttttccggggtgagggtaggtgtcaggggaggggtgggttatgaaaagtgcatggaggtgactgttcttgtgtggactgagcctggaagtgacccctgggaagatggggctgggtcccactctccaccctagggacaccttcatgtgagtgagcggctggggtggagtggcgaacctgacgccaggcgggtgtgggcccaagggctgcgtgcctggctgagcccagctcccctgtgtggagatgagtgtgccccatgccaggtgcacgttaggatcacctggagctattttaacaaacgctaaccccccctcccccatccagctggggtcttcaccccccaggggtgccttgcaaatcatgagatacaatgcttttcattggtagatgcacgtattagctcccgtatgaaccgtattactgaatttgaatctgaaaaataccaaaatgcaaacattgtttttaaattaattgttttaaaagctaatgaacgaattaagacaaattgcatcaatttagtggtttcttaaatcgtggtttgcaggtagttcagttttattaaaattcatttgtgagtcgctgaacgacttacattatgtaaaatatatcagacagtaaatgaattgtggc

SEQ ID NO: 10 - protein, 619 aa, Homo Sapien
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 11 - DNA, 2564 bp, Homo Sapien
gatttcagttgaaagatgtgtttttgtgagtagagcaccgcagaagaactgaagactgttgtgtgctccccgcagaaggggctaccatgatcctttcctcctataacaccatccagtcggttttctgttgctgctgttgctgttcagtgcagaagcgacaaatgagaacacagataagcctgagcacagatgaagagcttccagaaaaatacacccagcatcgcaggccgtggctcagccaattgtcaaataagaagcaatccaacacgggccgtgtgcagccgtcaaaacgaaagccactgcctcccctcccaccctctgaggttgctgaagagaagatccaagtcaaggcactttatgattttctgcccagagaaccctgtaatttagccttaaggagagcagaagaatacctgatactggagaaatacaatcctcactggtggaaggcaagagaccgtttggggaatgaaggcttaatcccaagcaactatgtgactgaaaacaaaataactaatttagaaatatatgagtggtaccatagaaacattaccagaaatcaggcagaacatctattgagacaagagtctaaagaaggtgcatttattgtcagagattcaagacatttaggatcctacacaatttccgtatttatgggagctagaagaagtacggaggctgccataaaacattatcagataaaaaagaatgactcaggacagtggtatgtggctgaaagacacgcctttcaatcaatccctgagttaatctggtatcaccagcacaatgcagccggtctcatgactcgtctccgatatccagttgggctgatgggcagttgtttaccagccacagctgggtttagctacgaaaagtgggagatagatccatctgagttggcttttataaaggagattggaagcggtcagtttggagtggtccatttaggtgaatggcggtcacatatccaggtagctatcaaggccatcaatgaaggctccatgtctgaagaggatttcattgaagaggccaaagtgatgatgaaattatctcattcaaagctagtgcaactttatggagtctgtatacagcggaagcccctttacattgtgacagagttcatggaaaatggctgcctgcttaactatctcagggagaataaaggaaagcttaggaaggaaatgctactgagtgtatgccaggatatatgtgaaggaatggaatatctggagaggaatggctatattcatagggatttggcggcaaggaattgtttggtcagttcaacatgcatagtaaaaatttcagactttggaatgacaaggtacgttttggatgatgagtatgtcagttcttttggagccaagttcccaatcaagtggtcccctcctgaagtttttcttttcaataagtacagcagtaaatctgatgtctggtcatttggagttttaatgtgggaagtttttacagaaggaaaaatgccttttgaaaataagtcaaatttgcaagtcgtggaagctatttctgaaggcttcaggctatatcgccctcacctggcaccaatgtccatatatgaagtcatgtacagctgctggcatgagaaacctgaaggccgccctacatttgcggagctgctgcgggctgtcacagagattgcggaaacctggtgaccggaaacagaatgccaacccaaagagtcatcttgcaaaactgtcatttattgtgaatatcttcaccatatggggtcacttatggtgaatatctttcttcagagttgctgactcttgaaaacagtgcaaagatcacagtttttaaaagttttaaaaatttaagaatattcacacaatcgtttttctatgtgtgagagggatttgcacactcttatttttctgtaaaatatttcacatcccaaatgtgaagaagtgaaaaagacttcgcagcagtcttcattgtggtgctcttcatgatcatagccccaggaacccttgaggttcttcttcacaaggctgagagtgcttccttcttgaagacgagtgtcattcatcacttcagtgatccatgcatagaatatgaaaataaattcttccaactcatgggataaaggggactcccttgaagaatttcatgtttttgggctgtatagctctttacagaaaatgcacctttataaatcacatgaatgttagtattctggaaatgtcttttgttaatataatcttcccatgttatttaacaaattgtttttgcacatatctgattatattgaaagcagtttttttgcattcgagttttaaacactgttataaaatgtagccaaagctcacctttgaacagatcccggtgacattctatttccaggaaaatccggaacctgattttagttctgtgattttacactttttacatgtgagattggacagtttcagaggccttattttgtcatactaagtgtctcctgtaattttcaggaagatgatttgttctttccagaagaggagacaaaagcaagatagccaaatgtgacatcaagctccattgtttcggaaatccaggattttgaattc

SEQ ID NO: 12 - protein, 527 aa, Homo Sapien
MILSSYNTIQSVFCCCCCCSVQKRQMRTQISLSTDEELPEKYTQHRRPWLSQLSNKKQSNTGRVQPSKRKPLPPLPPSEVAEEKIQVKALYDFLPREPCNLALRRAEEYLILEKYNPHWWKARDRLGNEGLIPSNYVTENKITNLEIYEWYHRNITRNQAEHLLRQESKEGAFIVRDSRHLGSYTISVFMGARRSTEAAIKHYQIKKNDSGQWYVAERHAFQSIPELIWYHQHNAAGLMTRLRYPVGLMGSCLPATAGFSYEKWEIDPSELAFIKEIGSGQFGVVHLGEWRSHIQVAIKAINEGSMSEEDFIEEAKVMMKLSHSKLVQLYGVCIQRKPLYIVTEFMENGCLLNYLRENKGKLRKEMLLSVCQDICEGMEYLERNGYIHRDLAARNCLVSSTCIVKISDFGMTRYVLDDEYVSSFGAKFPIKWSPPEVFLFNKYSSKSDVWSFGVLMWEVFTEGKMPFENKSNLQVVEAISEGFRLYRPHLAPMSIYEVMYSCWHEKPEGRPTFAELLRAVTEIAETW

SEQ ID NO: 13 - DNA, 2562 bp, Homo Sapien
cagtttctggagcaaattcagtttgccttcctggatttgtaaattgtaatgacctcaaaactttagcagttcttccatctgactcaggtttgcttctctggcggtcttcagaatcaacatccacacttccgtgattatctgcgtgcattttggacaaagcttccaaccaggatacgggaagaagaaatggctggtgatctttcagcaggtttcttcatggaggaacttaatacataccgtcagaagcagggagtagtacttaaatatcaagaactgcctaattcaggacctccacatgataggaggtttacatttcaagttataatagatggaagagaatttccagaaggtgaaggtagatcaaagaaggaagcaaaaaatgccgcagccaaattagctgttgagatacttaataaggaaaagaaggcagttagtcctttattattgacaacaacgaattcttcagaaggattatccatggggaattacataggccttatcaatagaattgcccagaagaaaagactaactgtaaattatgaacagtgtgcatcgggggtgcatgggccagaaggatttcattataaatgcaaaatgggacagaaagaatatagtattggtacaggttctactaaacaggaagcaaaacaattggccgctaaacttgcatatcttcagatattatcagaagaaacctcagtgaaatctgactacctgtcctctggttcttttgctactacgtgtgagtcccaaagcaactctttagtgaccagcacactcgcttctgaatcatcatctgaaggtgacttctcagcagatacatcagagataaattctaacagtgacagtttaaacagttcttcgttgcttatgaatggtctcagaaataatcaaaggaaggcaaaaagatctttggcacccagatttgaccttcctgacatgaaagaaacaaagtatactgtggacaagaggtttggcatggattttaaagaaatagaattaattggctcaggtggatttggccaagttttcaaagcaaaacacagaattgacggaaagacttacgttattaaacgtgttaaatataataacgagaaggcggagcgtgaagtaaaagcattggcaaaacttgatcatgtaaatattgttcactacaatggctgttgggatggatttgattatgatcctgagaccagtgatgattctcttgagagcagtgattatgatcctgagaacagcaaaaatagttcaaggtcaaagactaagtgccttttcatccaaatggaattctgtgataaagggaccttggaacaatggattgaaaaaagaagaggcgagaaactagacaaagttttggctttggaactctttgaacaaataacaaaaggggtggattatatacattcaaaaaaattaattcatagagatcttaagccaagtaatatattcttagtagatacaaaacaagtaaagattggagactttggacttgtaacatctctgaaaaatgatggaaagcgaacaaggagtaagggaactttgcgatacatgagcccagaacagatttcttcgcaagactatggaaaggaagtggacctctacgctttggggctaattcttgctgaacttcttcatgtatgtgacactgcttttgaaacatcaaagtttttcacagacctacgggatggcatcatctcagatatatttgataaaaaagaaaaaactcttctacagaaattactctcaaagaaacctgaggatcgacctaacacatctgaaatactaaggaccttgactgtgtggaagaaaagcccagagaaaaatgaacgacacacatgttagagcccttctgaaaaagtatcctgcttctgatatgcagttttccttaaattatctaaaatctgctagggaatatcaatagatatttaccttttattttaatgtttcctttaattttttactatttttactaatctttctgcagaaacagaaaggttttcttctttttgcttcaaaaacattcttacattttactttttcctggctcatctctttattttttttttttttttttaaagacagagtctcgctctgttgcccaggctggagtgcaatgacacagtcttggctcactgcaacttctgcctcttgggttcaagtgattctcctgcctcagcctcctgagtagctggattacaggcatgtgccacccacccaactaatttttgtgtttttaataaagacagggtttcaccatgttggccaggctggtctcaaactcctgacctcaagtaatccacctgcctcggcctcccaaagtgctgggattacagggatgagccaccgcgcccagcctcatctctttgttctaaagatggaaaaaccacccccaaattttctttttatactattaatgaatcaatcaattcatatctatttattaaatttctaccgcttttaggccaaaaaaatgtaagatcgttctctgcctcacatagcttacaagccagctggagaaatatggtactcattaaaaaaaaaaaaaaaaagtgatgtacaacc

SEQ ID NO: 14 - protein, 551 aa, Homo Sapien
MAGDLSAGFFMEELNTYRQKQGVVLKYQELPNSGPPHDRRFTFQVIIDGREFPEGEGRSKKEAKNAAAKLAVEILNKEKKAVSPLLLTTTNSSEGLSMGNYIGLINRIAQKKRLTVNYEQCASGVHGPEGFHYKCKMGQKEYSIGTGSTKQEAKQLAAKLAYLQILSEETSVKSDYLSSGSFATTCESQSNSLVTSTLASESSSEGDFSADTSEINSNSDSLNSSSLLMNGLRNNQRKAKRSLAPRFDLPDMKETKYTVDKRFGMDFKEIELIGSGGFGQVFKAKHRIDGKTYVIKRVKYNNEKAEREVKALAKLDHVNIVHYNGCWDGFDYDPETSDDSLESSDYDPENSKNSSRSKTKCLFIQMEFCDKGTLEQWIEKRRGEKLDKVLALELFEQITKGVDYIHSKKLIHRDLKPSNIFLVDTKQVKIGDFGLVTSLKNDGKRTRSKGTLRYMSPEQISSQDYGKEVDLYALGLILAELLHVCDTAFETSKFFTDLRDGIISDIFDKKEKTLLQKLLSKKPEDRPNTSEILRTLTVWKKSPEKNERHTC

SEQ ID NO: 15 - DNA, 3866 bp, Homo Sapien
ggaattcctttttttttttttttgagatggagtttcactcttgttggccaggctggagtgcaatggcacaatctcagcttactgcaacctccgcctcccgggttcaagcgattctcctgcctcagcctctcaagtagctgggattacaggcatgtgccaccacccctggctaactaatttcttttctatttagtagagatggggtttcaccatgttggtcaggctggtcttgaactcctgacctcaggtgatccacttgccttggcctcccaaagtgctaggattacagccgtgaaactgtgcctggctgattctttttttgttgttggatttttgaaacagggtctcccttggtcgcccaggctggagtgcagtggtgcgatcttggctcactataacctccacctcctggtttcaagtgatcctcccactttagcctcctgagtagctgtgattacaggcgtgcaccaccacacccggctaatttttgtatttttattagagacagggtttcaccatgttggccaggctgttctcaaactcctggactcaagggatccgcctgcctccacttcccaaagtcccgagattacaggtgtgagtcaccatgcctgaccttataattcttaagtcattttttctggtccatttcttccttagggtcctcacaacaaatctgcattaggcggtacaataatccttaacttcatgattcacaaaaggaagatgaagtgattcatgatttagaaaggggaagtagtaagcccactgcacactcctggatgatgatcctaaatccagatacagtaaaaatggggtatgggaaggtagaatacaaaatttggtttaaattaattatctaaatatctaaaaacatttttggatacattgttgatgtgaatgtaagactgtacagacttcctagaaaacagtttgggttccatcttttcatttccccagtgcagttttctgtagaaatggaatccgaggatttaagtggcagagaattgacaattgattccataatgaacaaagtgagagacattaaaaataagtttaaaaatgaagaccttactgatgaactaagcttgaataaaatttctgctgatactacagataactcgggaactgttaaccaaattatgatgatggcaaacaacccagaggactggttgagtttgttgctcaaactagagaaaaacagtgttccgctaagtgatgctcttttaaataaattgattggtcgttacagtcaagcaattgaagcgcttcccccagataaatatggccaaaatgagagttttgctagaattcaagtgagatttgctgaattaaaagctattcaagagccagatgatgcacgtgactactttcaaatggccagagcaaactgcaagaaatttgcttttgttcatatatcttttgcacaatttgaactgtcacaaggtaatgtcaaaaaaagtaaacaacttcttcaaaaagctgtagaacgtggagcagtaccactagaaatgctggaaattgccctgcggaatttaaacctccaaaaaaagcagctgctttcagaggaggaaaagaagaatttatcagcatctacggtattaactgcccaagaatcattttccggttcacttgggcatttacagaataggaacaacagttgtgattccagaggacagactactaaagccaggtttttatatggagagaacatgccaccacaagatgcagaaataggttaccggaattcattgagacaaactaacaaaactaaacagtcatgcccatttggaagagtcccagttaaccttctaaatagcccagattgtgatgtgaagacagatgattcagttgtaccttgttttatgaaaagacaaacctctagatcagaatgccgagatttggttgtgcctggatctaaaccaagtggaaatgattcctgtgaattaagaaatttaaagtctgttcaaaatagtcatttcaaggaacctctggtgtcagatgaaaagagttctgaacttattattactgattcaataaccctgaagaataaaacggaatcaagtcttctagctaaattagaagaaactaaagagtatcaagaaccagaggttccagagagtaaccagaaacagtggcaagctaagagaaagtcagagtgtattaaccagaatcctgctgcatcttcaaatcactggcagattccggagttagcccgaaaagttaatacagagcagaaacataccacttttgagcaacctgtcttttcagtttcaaaacagtcaccaccaatatcaacatctaaatggtttgacccaaaatctatttgtaagacaccaagcagcaataccttggatgattacatgagctgttttagaactccagttgtaaagaatgactttccacctgcttgtcagttgtcaacaccttatggccaacctgcctgtttccagcagcaacagcatcaaatacttgccactccacttcaaaatttacaggttttagcatcttcttcagcaaatgaatgcatttcggttaaaggaagaatttattccatattaaagcagataggaagtggaggttcaagcaaggtatttcaggtgttaaatgaaaagaaacagatatatgctataaaatatgtgaacttagaagaagcagataaccaaactcttgatagttaccggaacgaaatagcttatttgaataaactacaacaacacagtgataagatcatccgactttatgattatgaaatcacggaccagtacatctacatggtaatggagtgtggaaatattgatcttaatagttggcttaaaaagaaaaaatccattgatccatgggaacgcaagagttactggaaaaatatgttagaggcagttcacacaatccatcaacatggcattgttcacagtgatcttaaaccagctaactttctgatagttgatggaatgctaaagctaattgattttgggattgcaaaccaaatgcaaccagatacaacaagtgttgttaaagattctcaggttggcacagttaattatatgccaccagaagcaatcaaagatatgtcttcctccagagagaatgggaaatctaagtcaaagataagccccaaaagtgatgtttggtccttaggatgtattttgtactatatgacttacgggaaaacaccatttcagcagataattaatcagatttctaaattacatgccataattgatcctaatcatgaaattgaatttcccgatattccagagaaagatcttcaagatgtgttaaagtgttgtttaaaaagggacccaaaacagaggatatccattcctgagctcctggctcatccatatgttcaaattcaaactcatccagttaaccaaatggccaagggaaccactgaagaaatgaaatatgttctgggccaacttgttggtctgaattctcctaactccattttgaaagctgctaaaactttatatgaacactatagtggtggtgaaagtcataattcttcatcctccaagacttttgaaaaaaaaaggggaaaaaaatgatttgcagttattcgtaatgtcagataggaggtataaaatatattggactgttatactcttgaatccctgtggaaatctacatttgaagacaacatcactctgaagtgttatcagcaaaaaaaattcagtgagattatctttaaaagaaaactgtaaaaatagcaaccacttatggcactgtatatattgtagacttgttttctctgttttatgctcttgtgtaatctacttgacatcattttactcttggaatagtgggtggatagcaagtatattctaaaaaactttgtaaataaagttttgtggctaaaatga

SEQ ID NO: 16 - protein, 841 aa, Homo Sapien
MNKVRDIKNKFKNEDLTDELSLNKISADTTDNSGTVNQIMMMANNPEDWLSLLLKLEKNSVPLSDALLNKLIGRYSQAIEALPPDKYGQNESFARIQVRFAELKAIQEPDDARDYFQMARANCKKFAFVHISFAQFELSQGNVKKSKQLLQKAVERGAVPLEMLEIALRNLNLQKKQLLSEEEKKNLSASTVLTAQESFSGSLGHLQNRNNSCDSRGQTTKARFLYGENMPPQDAEIGYRNSLRQTNKTKQSCPFGRVPVNLLNSPDCDVKTDDSVVPCFMKRQTSRSECRDLVVPGSKPSGNDSCELRNLKSVQNSHFKEPLVSDEKSSELIITDSITLKNKTESSLLAKLEETKEYQEPEVPESNQKQWQAKRKSECINQNPAASSNHWQIPELARKVNTEQKHTTFEQPVFSVSKQSPPISTSKWFDPKSICKTPSSNTLDDYMSCFRTPVVKNDFPPACQLSTPYGQPACFQQQQHQILATPLQNLQVLASSSANECISVKGRIYSILKQIGSGGSSKVFQVLNEKKQIYAIKYVNLEEADNQTLDSYRNEIAYLNKLQQHSDKIIRLYDYEITDQYIYMVMECGNIDLNSWLKKKKSIDPWERKSYWKNMLEAVHTIHQHGIVHSDLKPANFLIVDGMLKLIDFGIANQMQPDTTSVVKDSQVGTVNYMPPEAIKDMSSSRENGKSKSKISPKSDVWSLGCILYYMTYGKTPFQQIINQISKLHAIIDPNHEIEFPDIPEKDLQDVLKCCLKRDPKQRISIPELLAHPYVQIQTHPVNQMAKGTTEEMKYVLGQLVGLNSPNSILKAAKTLYEHYSGGESHNSSSSKTFEKKRGKK

SEQ ID NO: 17 - DNA, 3650 bp, Homo Sapien
cggcggccgcggatcccggcggcgatccgacctcgcagtctccccaggtccgccagcagccggttcagccagaatactgggatcttcagtggcaggaggagtaatcagaagacggagatgaattttaacactattttggaggagattcttattaagaggtcacagcagaaaaagaagacatcgcccttaaactacaaagagagactttttgtacttacaaagtccatgctaacctactatgagggtcgagcagagaagaaatacagaaaggggtttattgatgtttcaaaaatcaagtgtgtggaaatagtgaagaatgatgatggtgtcattccctgtcaaaataagtatccatttcaggttgttcatgatgctaacacactttacatttttgcacctagtccacaaagcagggacctgtgggtgaagaagttaaaagaagaaataaagaacaacaataatattatgattaaatatcatcctaaattctggacagatggaagttatcagtgttgtagacaaactgaaaaattagcacccggatgtgaaaaatacaatctttttgagagcagtataagaaaagcactacctccagcaccagaaacaaagaagcgaaggcctcccccaccaattccactagaagaagaagataatagtgaagaaatcgttgtagccatgtatgatttccaagcagcagaaggacatgatctcagattagagagaggccaagagtatctcattttagaaaagaatgatgtgcattggtggagagcaagagataaatatgggaatgaaggatatatcccaagtaattacgtaacgggaaagaaatcaaacaacttagatcaatatgaatggtattgcagaaatatgaatagaagcaaggcagagcaactcctccgcagtgaagataaagaaggtggttttatggtaagggattccagtcaaccaggcttgtacacagtctccctttataccaagtttggaggagaaggttcatcgggttttaggcattatcatataaaggaaacaacaacatctccaaagaagtattacctagctgaaaaacatgcttttggctccattcctgagattattgaatatcataagcacaatgcagcaggacttgtcaccaggcttcggtacccagttagtgtgaaagggaagaatgcacccaccactgcaggattcagctatgagaaatgggagattaacccttcagaactgacctttatgagggaattgggaagtggactgtttggagtggtgaggcttggcaaatggcgagcccagtacaaagtcgcaatcaaagctattcgggaaggtgcaatgtgcgaggaggactttatagaagaagctaaagtgatgatgaagctgacacacccgaagttagtgcagctttatggtgtgtgcacccagcagaaaccaatatacattgttactgagttcatggaaaggggctgccttctgaatttcctccgacagagacaaggtcatttcagtagagacgtactgctgagcatgtgtcaggatgtgtgtgaagggatggagtatctggagagaaacagcttcatccacagagatctggctgccagaaattgtctagtaagtgaggcgggagttgtaaaagtatctgattttggaatggccaggtattttctggatgatcagtacacaagttcttctggtgctaagtttcctgtgaagtggtgtccacctgaagtgtttaattacagccgcttcagcagcaaatcagatgtctggtcatttggtgttttaatgtgggaagtattcacggaaggcagaatgccttttgaaaaatacaccaattatgaagtggtaaccatggttactcgaggccaccgactctaccagccgaagttggcgtccaactatgtgtatgaggtgatgctgagatgttggcaggagaaaccagagggaaggccttctttcgaagatctgctgcgcacaatagatgaactagttgaatgtgaagaaacttttggaagataagtgatgtgtgaccagtggctcccagattcccaagcacaaggaaggatgggcattttgtggcttttaatttattgagcacttggacatgtagatcattttacttatacagtggaaacacataaataatttgcttctagaccagcctctgtctagacttgcttctagacagaatctcccagagtgtggaaatgttgccttagaaatggtgattaaaatcactcatttctattcattcctcaggcacttgagtgacagttgtttaccaggcactgtgtgtagccccagggtttggccattcaggggtgcacacatgggaccatgttagctgatgccagttgaaggccagggtatttgggaaggggaagggtattagagtcatgaccaagcaacccttctttttccctttgacttctacagaaatctgggcctgagacattgtctacaattgggttctagatacatcaggaacccatcttggataaataaatacctatcttttgttttgaaaacatctcagttttcaagactgctcttagtattacatgaacaatatttgtatgctgtatatattgtaaatatatataatatataaagttatatatttatgagaaacacgaattgtcttttaattgaaacttttaatcctgtagtataggagttcaccttcttaggactagagactgtgccttatagctgttaattcatttccccctgaacatcaaatatgcctgaagagaagaaagtctagattcttctatgagtaacgccccctcctcactcaggtaaatgtgtctggggatgcctgtccagcttaaccacgtgcatttggcctatgtaatcctgcccatggtggccgcagctaatcagaatcagatggaaaattaaaccgggtaatctacttctaagccttaagaatattccctgggacacagacactataattggaagtgctgagctctggggcagaaggatcaggtgaccttcgcaacaaagtttgcccccacctcacataggacccggaagcagcctgagctgtggcggaggatccaggaagctacggagagaagcagccagcatggtgttccgtgcctcccggacgtttttcaggaggcctggttggacttgggttcctggatggtgggattgttgtacagcctctcaggagaccctgctgtcaagactgtgtgtgtggatttcccacccttagaagctctactaagacatcaacggaattagggccttcctttttgccttgtgagcgccaaggaaaagaaactatctcggtcacgtgagcgccacgaaagaaactgtatcagtcatccagagaccgtttattgcccaacacgttattcttgctgttggtggggtaactagccgaggaagacacagcgccttcccttcaggagttgcgtctcctctgcaggccacgatggtctgctctggagcattgggtgaacacacaggctggctgctctgggcagcgccttcactctgaccctggagaaccatttcatttcatcctggtcagtctagagtctgtgcaccaggcagtccatccactgaaggctgtgtttattcttttcctgtgcccctcataatggaagaaagtaaactgcttatcccgagccttaaaaaaa

SEQ ID NO: 18 - protein, 631 aa, Homo Sapien
MNFNTILEEILIKRSQQKKKTSPLNYKERLFVLTKSMLTYYEGRAEKKYRKGFIDVSKIKCVEIVKNDDGVIPCQNKYPFQVVHDANTLYIFAPSPQSRDLWVKKLKEEIKNNNNIMIKYHPKFWTDGSYQCCRQTEKLAPGCEKYNLFESSIRKALPPAPETKKRRPPPPIPLEEEDNSEEIVVAMYDFQAAEGHDLRLERGQEYLILEKNDVHWWRARDKYGNEGYIPSNYVTGKKSNNLDQYEWYCRNMNRSKAEQLLRSEDKEGGFMVRDSSQPGLYTVSLYTKFGGEGSSGFRHYHIKETTTSPKKYYLAEKHAFGSIPEIIEYHKHNAAGLVTRLRYPVSVKGKNAPTTAGFSYEKWEINPSELTFMRELGSGLFGVVRLGKWRAQYKVAIKAIREGAMCEEDFIEEAKVMMKLTHPKLVQLYGVCTQQKPIYIVTEFMERGCLLNFLRQRQGHFSRDVLLSMCQDVCEGMEYLERNSFIHRDLAARNCLVSEAGVVKVSDFGMARYFLDDQYTSSSGAKFPVKWCPPEVFNYSRFSSKSDVWSFGVLMWEVFTEGRMPFEKYTNYEVVTMVTRGHRLYQPKLASNYVYEVMLRCWQEKPEGRPSFEDLLRTIDELVECEETFGR

SEQ ID NO: 19 - DNA, 1782 bp, Homo Sapien
tagcgtgccatggcctgctacatctaccagctgccctcctgggtgctggacgacctgtgccgcaacatggacgcgctcagcgagtgggactggatggagttcgcctcctacgtgatcacagacctgacccagctgcggaagatcaagtccatggagcgggtgcagggtgtgagcatcacgcgggagctgctgtggtggtggggcatgcggcaggccaccgtccagcaacttgtggacctcctgtgccgcctggagctstaccgggctgcccagatcatcctgaactggaaaccggctcctgaaatcaggtgtcccattccagccttccctgactctgtgaagccagaaaagcctttggcagcttctgtaagaaaggctgaggatgaacaggaagaggggcagcctgtgaggatggccacctttccaggcccagggtcctctccagccagagcccaccagccggcctttctccagcctcctgaagaagatgcccctcattccttgagaagcgacctccccacttcgtctgattcaaaggacttcagcacctccattcctaagcaggaaaaacttttgagcttggctggagacagccttttctggagtgaggcagacgtggtccaggcaaccgatgacttcaatcaaaaccgcaaaatcagccaggggacctttgctgacgtctacagagggcacaggcacgggaagccattcgtcttcaagaagctcagagagacagcctgttcaagtccaggatcaatcgaaagattcttccaggcagagttgcagatttgtcttagatgctgccaccccaatgtcttacctgtgctgggcttctgtgctgcaagacagtttcacagcttcatctacccctacatggcaaatggttccctacaggacagactgcagggtcagggtggctcggaccccctcccctggccccagcgtgtcagcatctgctcagggctgctctgtgccgtcgagtacctgcatggtctggagatcatccacagcaacgtcaagagctctaatgtcttgctggaccaaaatctcacccccaaacttgctcacccaatggctcatctgtgtcctgtcaacaaaaggtcaaaatacaccatgatgaagactcacctgctccggacgtcagccgcgtatctgccagaggatttcatccgggtggggcagctgacaaagcgagtggacatcttcagctgtggaatagtgttggccgaggtcctcacgggcatccctgcaatggataacaaccgaagcccggtttacctgaaggacttactcctcagtgaaattccaagcagcaccgcctcgctctgctccaggaagacgggcgtggagaacgtgatggcaaaggagatctgccagaagtacctggagaagggcgcagggaggcttccggaggactgcgccgaggccctggccacggctgcctgcctgtgcctgcggaggcgtaacaccagcctgcaggaggtgtgtggctctgtggctgctgtggaagagcggctccgaggtcgggagacgttgctcccttggagtgggctttctgagggtacaggctcttcttccaacaccccagaggaaacagacgacgttgacaattccagccttgatgcctcctcctccatgagtgtggcaccctgggcaggggctgccaccccacttctccccacagagaatggggaaggaaggctgcgggtcatcgtgggaagggaggctgactcctcctctgaggcctgtgttggcctggagcctccccaggatgttacataa

SEQ ID NO: 20 - protein, 590 aa, Homo Sapien [sequence omitted]

SEQ ID NO: 21 - DNA, 2408 bp, Homo Sapien
ccgcgcctcctcggccgcctgtcgggcatgaaaaccaaattctgcaccgggggcgaggcggagccctcgccgctcgggctgctgctgagctgcggtagcggcagcgcggccccggcgcccggcgtggggcagcagcgcgacgccgccagcgacctcgagtccaagcagctggcgccaacagccgcgctcgcgctgccccctccgccgccgctgccgctgccgctgccgctgccccagcccccgccgccgcagccgcccgcagacgagcagccggagccccgggcgcggcgcagggcctatctgtggtgcaaggagttcctgcccggcgcctggcggggcctccgcgaggacgagttccacatcagtgtcatcagaggcggccttagcaacatgctgttccagtgctccctacctgacaccacagccacccttggtgatgagcctcggaaagtgctcctgcggctgtatggagcgattttgcagatgaggtcctgtaataaagagggatccgaacaagctcagaaagaaaatgaatttcaaggggctgaggccatggttctggagagcgttatgtttgccattctcgcagagaggtcacttgggccaaaactctatggcatctttccccaaggccgactggagcagttcatcccgagccggcgattagatactgaagaattaagtttgccagatatttctgcagaaatcgccgagaaaatggctacatttcatggtatgaaaatgccattcaataaggaaccaaaatggctttttggcacaatggaaaagtatctaaaggaagtgctgagaattaaatttactgaggaatccagaattaaaaagctccacaaattgctcagttacaatctgcccttggaactggaaaacctgagatcattgcttgaatctactccatctccagttgtattttgtcataatgactgtcaagaaggtaatatcttgttgctggaaggccgagagaattctgaaaaacagaaactgatgctcattgatttcgaatacagcagttacaattacaggggattcgacattggaaatcacttctgtgagtggatgtatgattatagctatgaaaaatacccttttttcagagcaaacatccggaagtatcccaccaagaaacaacagctccattttatttccagttacttgcctgcattccaaaatgactttgaaaacctcagtactgaagaaaaatccattataaaagaagaaatgttgcttgaagttaataggtttgcccttgcatctcatttcctctggggactgtggtccattgtacaagccaagatttcatctattgaatttgggtacatggactacgcccaagcaaggtttgatgcctatttccaccagaagaggaagcttggggtgtgactgtggggaggactccatccacctcatcactggactgcatggggaggcagcagagcgcggtcccctctgtgcttcgactactgctcctgtggcaggaggctttgggtggctcactactgaacacatgtgtatgatactaaagacggtattaaaatggagcgacgtttatttcatctcttgtttacgatttcactaggactcagaaacgagatcgggaagacgaaatatagtgcaatagtgcaacatctctgaatccttttaatctagagaaggcatttcatatttgggggctaaggtttccagtcagatgaggcaaacagcaagagtaagcagtgttacttgcaggtactttggttaatgttgactttaaattttcatgaatgtgctggtgaacactgtgaccaggcttttgtagatggcgactgtgttatagacggtgctcactcccaagggacagcaagtgagcagagatgtactgcaaagtcgccagtcactgcgtgcaaggtggcctctgcctggggccgtccagaagctgctcctttaccctcttggtcccatggctgaagcggagcagctggattgctctggagcagccaaggccgccactgtggagacagagctctcccctcctgctgggcgtgtgtgacactgtagagtttcactgtactcgatgtgacttctcccctgcccttcctcctgatggagtgtgcagacagccatgcgtggccacgggggcagtgtgaggacctccctgtctcccgctcccctcccagggagcagctgcttgacctagctctttgggcctctcctgccctctgctctgcctggagtgtcggatcctgtgagtaggctgggcctcccctgggcagggttctccaaggccggtttcccggcccttaccaaacctgatgcccctgacatcatcattcttgtgggagacagcagcctgtatgtggtgtggggcgtggatcgagtgtagctgtgaaatccatatatatgaaatgtccaat

SEQ ID NO: 22 - protein, 456 aa, Homo Sapien
MKTKFCTGGEAEPSPLGLLLSCGSGSAAPAPGVGQQRDAASDLESKQLAPTAALALPPPPPLPLPLPLPQPPPPQPPADEQPEPRARRRAYLWCKEFLPGAWRGLREDEFHISVIRGGLSNMLFQCSLPDTTATLGDEPRKVLLRLYGAILQMRSCNKEGSEQAQKENEFQGAEAMVLESVMFAILAERSLGPKLYGIFPQGRLEQFIPSRRLDTEELSLPDISAEIAEKMATFHGMKMPFNKEPKWLFGTMEKYLKEVLRIKFTEESRIKKLHKLLSYNLPLELENLRSLLESTPSPVVFCHNDCQEGNILLLEGRENSEKQKLMLIDFEYSSYNYRGFDIGNHFCEWMYDYSYEKYPFFRANIRKYPTKKQQLHFISSYLPAFQNDFENLSTEEKSIIKEEMLLEVNRFALASHFLWGLWSIVQAKISSIEFGYMDYAQARFDAYFHQKRKLGV

SEQ ID NO: 23 - DNA, 3707 bp, Homo Sapien
cccccattcgcatctaacaaggaatctgcgccccagagagtcccggacgccgccggtcggtgcccggcgcgccgggccatgcagcgacggccgccgcggagctccgagcagcggtagcgcccccctgtaaagcggttcgctatgccgggaccactgtgaaccctgccgcctgccggaacactcttcgctccggaccagctcagcctctgataagctggactcggcacgcccgcaacaagcaccgaggagttaagagagccgcaagcgcagggaaggcctccccgcacgggtgggggaaagcggccggtgcagcgcggggacaggcactcgggctggcactggctgctagggatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaaggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggctagggcccttttccccagaccgatccttcccaacgtactcctcagacgggctgagaggatgaacatcttttaactgccgctggaggccaccaagctgctctccttcactctgacagtattaacatcaaagactccgagaagctctcgagggaagcagtgtgtacttcttcatccatagacacagtattgacttctttttggcattatctctttctctctttccatctcccttggttgttcctttttctttttttaaattttctttttcttcttttttttcgtcttccctgcttcacgattcttaccctttcttttgaatcaatctggcttctgcattactattaactctgcatagacaaaggccttaacaaacgtaatttgttatatcagcagacactccagtttgcccaccacaactaacaatgccttgttgtattcctgcctttgatgtggatgaaaaaaagggaaaacaaatatttcacttaaactttgtcacttctgctgtacagatatcgagagtttctatggattcacttctatttatttattattattactgttcttattgtttttggatggcttaagcctgtgtataaaaaagaaaacttgtgttcaatctgtgaagcctttatctatgggagattaaaaccagagagaaagaagatttattatgaaccgcaatatgggaggaacaaagacaaccactgggatcagctggtgtcagtccctacttaggaaatactcagcaactgttagctgggaagaatgtattcggcaccttcccctgaggacctttctgaggagtaaaaagactactggcctctgtgccatggatgattcttttcccatcaccagaaatgatagcgtgcagtagagagcaaagatggctt

SEQ ID NO: 24 - protein, 822 aa, Homo Sapien
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 25 - DNA, 2180 bp, Homo Sapien
gtgaaattctgctccggacatgtcgggccctcgcgccggcttctaccggcaggagctgaacaagaccgtgtgggaggtgccgcagcggctgcaggggctgcgcccggtgggctccggcgcctacggctccgtctgttcggcctacgacgcccggctgcgccagaaggtggcggtgaagaagctgtcgcgccccttccagtcgctgatccacgcgcgcagaacgtaccgggagctgcggctgctcaagcacctgaagcacgagaacgtcatcgggcttctggacgtcttcacgccggccacgtccatcgaggacttcagcgaagtgtacttggtgaccaccctgatgggcgccgacctgaacaacatcgtcaagtgccaggcgggcgcccatcagggtgcccgcctggcacttgacgagcacgttcaattcctggtttaccagctgctgcgcgggctgaagtacatccactcggccgggatcatccaccgggacctgaagcccagcaacgtggctgtgaacgaggactgtgagctcaggatcctggatttcgggctggcgcgccaggcggacgaggagatgaccggctatgtggccacgcgctggtaccgggcacctgagatcatgctcaactggatgcattacaaccaaacagtggatatctggtccgtgggctgcatcatggctgagctgctccagggcaaggccctcttcccgggaagcgactacattgaccagctgaagcgcatcatggaagtggtgggcacacccagccctgaggttctggcaaaaatctcctcggaacacgcccggacatatatccagtccctgccccccatgccccagaaggacctgagcagcatcttccgtggagccaaccccctggccatagacctccttggaaggatgctggtgctggacagtgaccagagggtcagtgcagctgaggcactggcccacgcctacttcagccagtaccacgaccccgaggatgagccagaggccgagccatatgatgagagcgttgaggccaaggagcgcacgctggaggagtggaaggagctcacttaccaggaagtccttagcttcaagcccccagagccaccgaagccacctggcagcctggagattgagcagtgaggtgctgcccagcagcccctgagagcctgtggaggggcttgggcctgcacccttccacagctggcctggtttcctcgagaggcacctcccacactcctatggtcacagacttctggcctaggacccctcgccttcaggagaatctacacgcatgtatgcatgcacaaacatgtgtgtacatgtgcttgccatgtgtaggagtctgggcacaagtgtccctgggcctaccttggtcctcctgtcctcttctggctactgcactctccactgggacctgactgtggggtcctagatgccaaaggggttcccctgcggagttcccctgtctgtcccaggccgacccaagggagtgtcagccttgggctctcttctgtcccagggctttctggagggcgcgctggggccgggaccccgggagactcaaagggagaggtctcagtggttagagctgctcagcctggaggtagggcgctgtcttggtcactgctgagacccacaggtctaagaggagaggcagagccagtgtgccaccaggctgggcagggacaaccaccaggtgtcaaatgagaaaagctgcctggagtcttgtgttcacccgtgggtgtgtgtgggcacgtgtggatgagcgtgcactccccgtgttcatatgtcagggcacatgtgatgtggtgcgtgtgaatctgtgggcgcccaaggccagcagccatatctggcaagaagctggagccggggtgggtgtgctgttgccttccctctcctcggttcctgatgccttgaggggtgtttcagactggcggcaccgttgtggccctgcagccggagatctgaggtgctctggtctgtgggtcagtcctctttccttgtcccaggatggagctgatccagtaacctcggagacgggaccctgcccagagctgagttgggggtgtggctctgccctggaaagggggtgacctcttgcctcgaggggcccagggaagcctgggtgtcaagtgcctgcaccaggggtgcacaataaagggggttctctctcagaaaaaaa

SEQ ID NO: 26 - protein, 372 aa, Homo Sapien
MSGPRAGFYRQELNKTVWEVPQRLQGLRPVGSGAYGSVCSAYDARLRQKVAVKKLSRPFQSLIHARRTYRELRLLKHLKHENVIGLLDVFTPATSIEDFSEVYLVTTLMGADLNNIVKCQAGAHQGARLALDEHVQFLVYQLLRGLKYIHSAGIIHRDLKPSNVAVNEDCELRILDFGLARQADEEMTGYVATRWYRAPEIMLNWMHYNQTVDIWSVGCIMAELLQGKALFPGSDYIDQLKRIMEVVGTPSPEVLAKISSEHARTYIQSLPPMPQKDLSSIFRGANPLAIDLLGRMLVLDSDQRVSAAEALAHAYFSQYHDPEDEPEAEPYDESVEAKERTLEEWKELTYQEVLSFKPPEPPKPPGSLEIEQ

SEQ ID NO: 27 - DNA, 2032 bp, Homo Sapien
cgcctggaccatgtgaatggggccagagggctcccgggctgggcagggaccatgggctgtggctgcagctcacacccggaagatgactggatggaaaacatcgatgtgtgtgagaactgccattatcccatagtcccactggatggcaagggcacgctgctcatccgaaatggctctgaggtgcgggacccactggttacctacgaaggctccaatccgccggcttccccactgcaagacaacctggttatcgctctgcacagctatgagccctctcacgacggagatctgggctttgagaagggggaaccactccgcatcctggagcagagcggcgagtggtggaaggcgcagtccctgaccacgggccaggaaggcttcatccccttcaattttgtggccaaagcgaacagcctggagcccgaaccctggttcttcaagaacctgagccgcaaggacgcggagcggcagctcctggcgcccgggaacactcacggctccttcctcatccgggagagcgagagcaccgccgggtccttttcactgtcggtccgggacttcgaccaaaaccagggagaggtggtgaaacattacaagatccgtaatctggacaacggtggcttctacatctcccctcgaatcacttttcccggcctgcatgaactggtccgccattacaccaatgcttcagatgggctgtgcacacggttgagccgcccctgccagacccagaagccccagaagccgtggtgggaggacgagtgggaggttcccagggagacgctgaagctggtggagcggctgggggctggacagttcggggaggtgtggatggggtactacaacgggcacacgaaggtggcggtgaagagcctgaagcagggcagcatgtccccggacgccttcctggccgaggccaacctcatgaagcagctgcaacaccagcggctggttcggctctacgctgtggtcacccaggagcccatctacatcatcactgaatacatggagaatgggagtctagtggattttctcaagaccccttcaggcatcaagttgaccatcaacaaactcctggacatggcagcccaaattgcagaaggcatggcattcattgaagagcggaattatattcatcgtgaccttcgggctgccaacattctggtgtctgacaccctgagctgcaagattgcagactttggcctagcacgcctcattgaggacaacgagtacacagccagggagggggccaagtttcccattaagtggacagcgccagaagccattaactacgggacattcaccatcaagtcagatgtgtggtcttttgggatcctgctgacggaaattgtcacccacggccgcatcccttacccagggatgaccaacccggaggtgattcagaacctggagcgaggctaccgcatggtgcgccctgacaactgtccagaggagctgtaccaactcatgaggctgtgctggaaggagcgcccagaggaccggcccacctttgactacctgcgcagtgtgctggaggacttcttcacggccacagagggccagtaccagcctcagccttgagaggaggccttgagaggccctggggttctccccctttctctccagcctgacttggggagatggagttcttgtgccatagtcacatggcctatgcacatatggactctgcacatgaatcccacccacatgtgacacatatgcaccttgtgtctgtacacgtgtcctgtagttgcgtggactctgcacatgtcttgtgcatgtgtagcctgtgcatgtatgtcttggacactgtacaaggtacccctttctggctctcccatttcctgagaccaccagagagaggggagaagcctgggattgacagaagcttctgcccacctacttttctttcctcagatcatccagaagttcctcaagggccaggactttatctaatacctctgtgtgctcctccttggtgcctggcctggcacacatcaggagttcaataaatgtctgttgatgactgccg

SEQ ID NO: 28 - protein, 509 aa, Homo Sapien
MGCGCSSHPEDDWMENIDVCENCHYPIVPLDGKGTLLIRNGSEVRDPLVTYEGSNPPASPLQDNLVIALHSYEPSHDGDLGFEKGEPLRILEQSGEWWKAQSLTTGQEGFIPFNFVAKANSLEPEPWFFKNLSRKDAERQLLAPGNTHGSFLIRESESTAGSFSLSVRDFDQNQGEVVKHYKIRNLDNGGFYISPRITFPGLHELVRHYTNASDGLCTRLSRPCQTQKPQKPWWEDEWEVPRETLKLVERLGAGQFGEVWMGYYNGHTKVAVKSLKQGSMSPDAFLAEANLMKQLQHQRLVRLYAVVTQEPIYIITEYMENGSLVDFLKTPSGIKLTINKLLDMAAQIAEGMAFIEERNYIHRDLRAANILVSDTLSCKIADFGLARLIEDNEYTAREGAKFPIKWTAPEAINYGTFTIKSDVWSFGILLTEIVTHGRIPYPGMTNPEVIQNLERGYRMVRPDNCPEELYQLMRLCWKERPEDRPTFDYLRSVLEDFFTATEGQYQPQP

SEQ ID NO: 29 - DNA, 3007 bp, Homo Sapien
  misc_feature (1)...(3007): n = A,T,C or G
gtcgacccacgcgtccgcggacgcgtgggcggacgcgtgggcgacccacgcgtccggtgatggtgcctcaaagcagtaactttttgcttagagcttgagagtcaaagttaaggacccacatgtatacttcggctctagcgagtctaaggatgataatatggatacaaaatctattctagaagaacttcttctcaaaagatcacagcaaaagaagaaaatgtcaccaaataattacaaagaacggctttttgttttgaccaaaacaaacctttcctactatgaatatgacaaaatgaaaaggggcagcagaaaaggatccattgaaattaagaaaatcagatgtgtggagaaagtaaatctcgaggagcagacgcctgtagagagacagtacccatttcagattgtctataaagatgggcttctctatgtctatgcatcaaatgaagagagccgaagtcagtggttgaaagcattacaaaaagagataaggggtaacccccacctgctggtcaagtaccatagtgggttcttcgtggacgggaagttcctgtgttgccagcagagctgtaaagcagccccaggatgtaccctctgggaagcatatgctaatctgcatactgcagtcaatgaagagaaacacagagttcccaccttcccagacagagtgctgaagatacctcgggcagttcctgttctcaaaatggatgcaccatcttcaagtaccactctagcccaatatgacaacgaatcaaagaaaaactatggctcccagccaccatcttcaagtaccagtctagcgcaatatgacagcaactcaaagaaaatctatggctcccagccaaacttcaacatgcagtatattccaagggaagacttccctgactggtggcaagtaagaaaactgaaaagtagcagcagcagtgaagatgttgcaagcagtaaccaaaaagaaagaaatgtgaatcacaccacctcaaagatttcatgggaattccctgagtcaagttcatctgaagaagaggaaaacctggatgattatgactggtttgctggtaacatctccagatcacaatctgaacagttactcagacaaaagggaaaagaaggagcatttatggttagaaattcgagccaagtgggaatgtacacagtgtccttatttagtaaggctgtgaatgataaaaaaggaactgtcaaacattaccacgtgcatacaaatgctgagaacaaattatacctggcagaaaactactgttttgattccattccaaagcttattcattatcatcaacacaattcagcaggcatgatcacacggctccgccaccctgtgtcaacaaaggccaacaaggtccccgactctgtgtccctgggaaatggaatctgggaactgaaaagagaagagattaccttgttgaaggagctgggaagtggccagtttggagtggtccagctgggcaagtggaaggggcagtatgatgttgctgttaagatgatcaaggagggctccatgtcagaagatgaattctttcaggaggcccagactatgatgaaactcagccatcccaagctggttaaattctatggagtgtgttcaaaggaataccccatatacatagtgactgaatatataagcaatggctgcttgctgaattacctgaggagtcacggaaaaggacttgaaccttcccagctcttagaaatgtgctacgatgtctgtgaaggcatggccttcttggagagtcaccaattcatacaccgggacttggctgctcgtaactgcttggtggacagagatctctgtgtgaaagtatctgactttggaatgacaaggtatgttcttgatgaccagtatgtcagttcagtcggaacaaagtttccagtcaagtggtcagctccagaggtgtttcattacttcaaatacagcagcaagtcagacgtatgggcatttgggatcctgatgtgggaggtgttcagcctggggaagcagccctatgacttgtatgacaactcccaggtggttctgaaggtctcccagggccacaggctttaccggccccacctggcatcggacaccatctaccagatcatgtacagctgctggcacgagcttccagaaaagcgtcccacatttcagcaactcctgtcttccattgaaccacttcgggaaaaagacaagcattgaagaagaaattaggagtgctgataagaatgaatatagatgctggccagcattttcattcattttaaggaaagtagcaaggcataatgtaatttagctagtttttaatagtgttctctgtattgtctattatttagaaatgaacaaggcaggaaacaaaagattcccttgaaatttagatcaaattagtaattttgtttatgctgctcctgatataacactttccagcctatagcagaagcacattttcagactgcaatatagagactgtgttcatgtgtaaagactgagcagaactgaaaaattacttattggatattcattcttttctttatattgtcattgtcacaacaattaaatatactaccaagtaaaaaaaaaaaaaaaagggcggccgctctagagtatccctcgaggggcccaagcttacgcgtacccagctttcttgtacaaagtggtnnnnnnnnnnnnnnnnnnnnnnnnnnnnnnnnnnnnnnnnnnnnnnnnnnnnnnnnnnnnnnnnnnntgctagcttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatatgcttgctgcttgagagttttgcttactgagtatgatttatgaaaatattatacacaggagnnnnnnnnnnnnnnnnnnnnnnnnnnnnnnnnnnacagtcccaaggctcatttcaggcccctcagtcctcacagtctgttcatgatcataatcagccataccac

SEQ ID NO: 30 - protein, 697 aa, Homo Sapien [sequence omitted]

SEQ ID NO: 31 - DNA, 2588 bp, Homo Sapien
  misc_feature (1)...(2588): n = A,T,C or G
gggaggggtgcgaggctagccacgcaggcggggccctgggtcattttaaactctcagagtgaacgtcttgataggaccgacaagacgcatgacatgtacttagatagcttatcttagagccacactgagattggaacccgcaaaatatgccagggaggaaggtgagcaagggacacgacactcacccggataaacccaacaagcgcagcgaggctgtggggaaaccgganccctgcacaccgccgggggaaggtgggccnccgccaccaccgtggaagaacagcgcggangcaccccacgagatgagacggaactgccgtgagatccagcaatnccnactgtgggtctgacccaggatancggaaagcagggacgtgaacagccctcctcatgttcttgacaccgtcattctcagcagctcagctaaggcacagaggcagccgagcgtctgtcagcagagtcgtggctgagcagaacacgccacacgccacacgccacacgccacacgtgcaggattgctcaagatggaagggcacagtggaatatatatatatatttatatttttggcgagaccctggaggacacactgaatacaatggaataccatcccgcctttgaaaggaagggaaatcctggcacacgctgcaacaggagggagcttgaggacactgtggtgagtggagcacgtgagacacggaaggacacacgctgaagacacgcagagatgcccacccacgtggggaggtgacaggggagcccagcgcacagagacaaagtggaatggaggcctgggggctgggagcaaatgcggagcgagtgcttcctggggcagagtctccgtttgggaagatgagaaggttctgccgacggatgctggcgatggttgcagaagaatgtgaatgtgcccaatgctactgaaaaacggttacaatggaaacgccaccccagtgaccaccactgccccgtgggcctccctgggcctctccgccaagacctgcaacaacgtgtccttcgaagagagcaggatagtcctggtcgtggtgtacagcgcggtgtgcacgctgggggtgccggccaactgcctgactgcgtggctggcgctgctgcaggtactgcagggcaacgtgctggccgtctacctgctctgcctggcactctgcgagctgctgtacacaggcacgctgccactctgggtcatctatatccgcaaccagcaccgctggaccctaggcctgctggcctgcaaggtgaccgcctacatcttcttctgcaacatctacgtcagcatcctcttcctgtgctgcatctcctgcgaccgcttcgtggccgtggtgtacgcgctggagagtcggggccgccgccgccggaggaccgccatcctcatctccgcctgcatcttcatcctcgtcgggatcgttcactacccggtgttccagacggaagacaaggagacctgctttgacatgctgcagatggacagcaggattgccgggtactactacgccaggttcaccgttggctttgccatccctctctccatcatcgccttcaccaaccaccggattttcaggagcatcaagcagagcatgggcttaagcgctgcccagaaggccaaggtgaagcactcggccatcgcggtggttgtcatcttcctagtctgcttcgccccgtaccacctggttctcctcgtcaaagccgctgccttttcctactacagaggagacaggaacgccatgtgcggcttggaggaaaggctgtacacagcctctgtggtgtttctgtgcctgtccacggtgaacggcgtggctgaccccattatctacgtgctggccacggaccattcccgccaagaagtgtccagaatccataaggggtggaaagagtggtccatgaagacagacgtcaccaggctcacccacagcagggacaccgaggagctgcagtcgcccgtggcccttgcagaccactacaccttctccaggcccgtgcacccaccagggtcaccatgccctgcaaagaggctgattgaggagtcctgctgagcccactgtgtggcagggggatggcaggttgggggtcctggggccagcaatgtggttcctgtgcactgagcccaccagccacagtgcccatgtcccctctggaagacaaactaccaatttctcgttcctgaagccactccctccgtgaccactggccccangctttcccacatggaaggtggctgcatgccaaggggaagagcgacacctccaggcttccgggagcccanagagcatgtggcangcagtggggcctcttcatcatcancctgcctggctggctcccttggctgtgggcangtacacccctgctggcagaagtacctggtggctgccctgttcgcatcagtggcgatgactttatttgcggagcatttctgcaagcgttgcctggatgcggtggtgcattgtgggccctctgggctcctgcctcaaaatgtcagtgagcaccatgctggaagtcaccatcactgtggcagcgcccaggaaggcatagggcancctaccacctccaanggggcangcgccctcatctggggttgggt

SEQ ID NO: 32 - protein, 380 aa, Homo Sapien
MCPMLLKNGYNGNATPVTTTAPWASLGLSAKTCNNVSFEESRIVLVVVYSAVCTLGVPANCLTAWLALLQVLQGNVLAVYLLCLALCELLYTGTLPLWVIYIRNQHRWTLGLLACKVTAYIFFCNIYVSILFLCCISCDRFVAVVYALESRGRRRRRTAILISACIFILVGIVHYPVFQTEDKETCFDMLQMDSRIAGYYYARFTVGFAIPLSIIAFTNHRIFRSIKQSMGLSAAQKAKVKHSAIAVVVIFLVCFAPYHLVLLVKAAAFSYYRGDRNAMCGLEERLYTASVVFLCLSTVNGVADPIIYVLATDHSRQEVSRIHKGWKEWSMKTDVTRLTHSRDTEELQSPVALADHYTFSRPVHPPGSPCPAKRLIEESC

SEQ ID NO: 33 - DNA, 2261 bp, Homo Sapien
cccgggaggcggacgttgtagagagctgagatcgcaccactgcactccagcctgggtgacaaagcgagattctgtctcaaaaaaaaaaaaaaaaaaaaaaaaactataaacaatggatggacaagaaaatcatgctggtgtgcgaagctaatgtgtttccctctcttccagatgctggccaagaagagctgaaataagaaaacagcctagaacctaacactatttactgtaaaaatttttgcaccaggatggaaggggattcttaccacaatgcaaccaccgtcaatggcaccccagtaaatcaccagcctttggaacgccacaggttgtgggaagtcatcaccattgcagctgtgactgctgtggtaagcctgatcaccattgtgggcaatgtcttggtcatgatctccttcaaagtcaacagccagctcaagacagttaacaactattacctgctcagcttagcctgtgcagatctcatcattggaatcttctccatgaacctctacaccacctacatcctcatgggacgctgggctctcgggagtctggcttgtgacctttggcttgcactggactacgtggccagcaacgcttctgtcatgaaccttctggtgatcagttttgaccgttacttttccatcacaagacccttgacatatcgggccaagcgtactccgaaaagggctggcatcatgattggcttggcctggctgatctccttcatcctctgggccccagcaatcctctgctggcagtacttggttgggaagcggacagttccactggatgagtgccagatccagtttctctctgagcccaccatcacttttggcactgccattgctgccttctacatccctgtttctgtcatgaccatcctctactgtcgaatctaccgggaaacagagaagcgaaccaaggacctggctgacctccagggttctgactctgtgaccaaagctgagaagagaaagccagctcatagggctctgttcagatcctgcttgcgctgtcctcgacccaccctggcccagcgggaaaggaaccaggcctcctggtcatcctcccgcaggagcacctccaccactgggaagccatcccaagccactggcccaagcgccaattgggccaaagctgagcagctcaccacctgtagcagctacccttcctcagaggatgaggacaagcccgccactgaccctgtcctccaagtggtctacaagagtcagggtaaggaaagcccaggggaagaattcagtgctgaagagactgaggaaacttttgtgaaagctgaaactgaaaaaagtgactatgacaccccaaactaccttctgtctccagcagctgctcatagacccaagagtcagaaatgtgtggcctataagttccgattggtggtaaaagctgacgggaaccaggagaccaacaatggctgtcacaaggtgaaaatcatgccctgccccttcccagtggccaaggaaccttcaacgaaaggcctcaatcccaaccccagccatcaaatgaccaaacgaaagagagtggtcctagtcaaagagaggaaagcagcccagacactgagtgccattctcctggccttcatcatcacatggaccccgtataacatcatggtcctggtttctaccttctgtgacaagtgtgtcccagtcaccctgtggcacttgggctattggttgtgctatgtcaatagcactgtcaaccccatctgctatgccctctgcaacagaaccttcaggaagacctttaagatgctgcttctctgccgatggaaaaagaaaaaagtggaagagaagttgtactggcaggggaacagcaagctaccctgaaaagtcaacaactcctctcgaaagaacaatgaccacagtcaacatcctctgaggatgagcaagctgattctggtttgtatattttcaaaaagaagacatctcattttgagtccttgaagatttttgtaaaggctcaagtttggttgccaaatggaaggggccatagctgcagcaattgctgacatattaaatgactcttgcctatgaccaaggccatttgatgccaggggagtttgccaatgaagtaaagggataggctcatggcccttcacaagaggaagcacactgggtaacaatgaacagtgactcagggaacttatgccccttctgtaggaaacagcagagaccaggtggaaaccttttcctgtggaaacctgtcatagaattttgtgcaatatgtatgtgtctatgaag

SEQ ID NO: 34 - protein, 532 aa, Homo Sapien
MEGDSYHNATTVNGTPVNHQPLERHRLWEVITIAAVTAVVSLITIVGNVLVMISFKVNSQLKTVNNYYLLSLACADLIIGIFSMNLYTTYILMGRWALGSLACDLWLALDYVASNASVMNLLVISFDRYFSITRPLTYRAKRTPKRAGIMIGLAWLISFILWAPAILCWQYLVGKRTVPLDECQIQFLSEPTITFGTAIAAFYIPVSVMTILYCRIYRETEKRTKDLADLQGSDSVTKAEKRKPAHRALFRSCLRCPRPTLAQRERNQASWSSSRRSTSTTGKPSQATGPSANWAKAEQLTTCSSYPSSEDEDKPATDPVLQVVYKSQGKESPGEEFSAEETEETFVKAETEKSDYDTPNYLLSPAAAHRPKSQKCVAYKFRLVVKADGNQETNNGCHKVKIMPCPFPVAKEPSTKGLNPNPSHQMTKRKRVVLVKERKAAQTLSAILLAFIITWTPYNIMVLVSTFCDKCVPVTLWHLGYWLCYVNSTVNPICYALCNRTFRKTFKMLLLCRWKKKKVEEKLYWQGNSKLP

SEQ ID NO: 35 - DNA, 2372 bp, Homo Sapien
gggccgccgtcggcgcgctgggtgcgggaagggggctctggatttcggtccctcccctttttcctctgagtctcggaacgctccagctctcagaccctcttcctcccaggtaaaggccgggagaggagggcgcatctcttttccaggcaccccaccatgggcaatgcctccaatgactcccagtctgaggactgcgagacgcgacagtggcttcccccaggcgaaagcccagccatcagctccgtcatgttctcggccggggtgctggggaacctcatagcactggcgctgctggcgcgccgctggcggggggacgtggggtgcagcgccggccgcaggagctccctctccttgttccacgtgctggtgaccgagctggtgttcaccgacctgctcgggacctgcctcatcagcccagtggtactggcttcgtacgcgcggaaccagaccctggtggcactggcgcccgagagccgcgcgtgcacctacttcgctttcgccatgaccttcttcagcctggccacgatgctcatgctcttcgccatggccctggagcgctacctctcgatcgggcacccctacttctaccagcgccgcgtctcggcctccgggggcctggccgtgctgcctgtcatctatgcagtctccctgctcttctgctcgctgccgctgctggactatgggcagtacgtccagtactgccccgggacctggtgcttcatccggcacgggcggaccgcttacctgcagctgtacgccaccctgctgctgcttctcattgtctcggtgctcgcctgcaacttcagtgtcattctcaacctcatccgcatgcaccgccgaagccggagaagccgctgcggaccttccctgggcagtggccggggcggccccggggcccgcaggagaggggaaagggtgtccatggcggaggagacggaccacctcattctcctggctatcatgaccatcaccttcgccgtctgctccttgcctttcacgatttttgcatatatgaatgaaacctcttcccgaaaggaaaaatgggacctccaagctcttaggtttttatcaattaattcaataattgacccttgggtctttgccatccttaggcctcctgttctgagactaatgcgttcagtcctctgttgtcggatttcattaagaacacaagatgcaacacaaacttcctgttctacacagtcagatgccagtaaacaggctgacctttgaggtcagtagtttaaaagttcttagttatatagcatctggaagatcattttgaaattgttccctggagaaatgaaaacagtgtgtaaacaaaatgaagctgccctaataaaaaggagtatacaaacatttaagctgtggtcaaggctacagatgtgctgacaaggcacttcatgtaaagtgtcagaaggagctacaaaacctaccctcaatgagcatggtacttggcctttggaggaacaatcggctgcattgaagatccagctgcctattgatttaagctttcctgttgaatgacaaagtatgtggttttgtaatttgtttgaaaccccaaacagtgactgtactttctattttaatcttgctactaccgttatacacatatagtgtacagccagaccagattaaacttcatatgtaatctctaggaagtcaatatgtggaagcaaccaagcctgctgtcttgtgatcacttagcgaaccctttatttgaacaatgaagttgaaaatcataggcaccttttactgtgatgtttgtgtatgtgggagtactctcatcactacagtattactcttacaagagtggactcagtgggttaacatcagttttgtttactcatcctccaggaactgcaggtcaagttgtcaggttatttattttataatgtccatatgctaatagtgatcaagaagactttaggaatggttctctcaacaagaaataatagaaatgtctcaaggcagttaattctcattaatactcttattatcctatttctgggggaggatgtacgtggccatgtatgaagccaaatattaggcttaaaaactgaaaaatctggttcattcttcagatatactggaacccttttaaagttgatattggggccatgagtaaaatagattttataagatgactgtgttgtaccaaaattcatctgtctatattttatttaggggaacatggtttgactcatcttatatgggaaaccatgtagcagtgagtcatatcttaatatatttctaaatgtttggcatgtaaatgtaaactcagcatcaaaatatttcagtgaatttgcactgtttaatcatagttactgtgtaaactcatctgaaatgttacaaaaataaactataaaaca

SEQ ID NO: 36 - protein, 358 aa, Homo Sapien
MGNASNDSQSEDCETRQWLPPGESPAISSVMFSAGVLGNLIALALLARRWRGDVGCSAGRRSSLSLFHVLVTELVFTDLLGTCLISPVVLASYARNQTLVALAPESRACTYFAFAMTFFSLATMLMLFAMALERYLSIGHPYFYQRRVSASGGLAVLPVIYAVSLLFCSLPLLDYGQYVQYCPGTWCFIRHGRTAYLQLYATLLLLLIVSVLACNFSVILNLIRMHRRSRRSRCGPSLGSGRGGPGARRRGERVSMAEETDHLILLAIMTITFAVCSLPFTIFAYMNETSSRKEKWDLQALRFLSINSIIDPWVFAILRPPVLRLMRSVLCCRISLRTQDATQTSCSTQSDASKQADL

SEQ ID NO: 37 - DNA, 1200 bp, Homo Sapien
caagtggacctgtactgaaaatgggtccaataggtgcagaggctgatgagaaccagacagtggaagaaatgaaggtggaacaatacgggccacaaacaactcctagaggtgaactggtccctgaccctgagccagagcttatagatagtaccaagctgattgaggtacaagttgttctcatattggcctactgctccatcatcttgcttggggtaattggcaactccttggtgatccatgtggtgatcaaattcaagagcatgcgcacagtaaccaactttttcattgccaatctggctgtggcagatcttttggtgaacactctgtgtctaccgttcactcttacctataccttaatgggggagtggaaaatgggtcctgtcctgtgccacctggtgccctatgcccagggcctggcagtacaagtatccacaatcaccttgacagtaattgccctggaccggcacaggtgcatcgtctaccacctagagagcaagatctccaagcgaatcagcttcctgattattggcttggcctggggcatcagtgccctgctggcaagtcccctggccatcttccgggagtattcgctgattgagatcatcccggactttgagattgtggcctgtactgaaaagtggcctggcgaggagaagagcatctatggcactgtctatagtctttcttccttgttgatcttgtatgttttgcctctgggcattatatcattttcctacactcgcatttggagtaaattgaagaaccatgtcagtcctggagctgcaaatgaccactaccatcagcgaaggcaaaaaaccaccaaaatgctggtgtgtgtggtggtggtgtttgcggtcagctggctgcctctccatgccttccagcttgccgttgacattgacagccaggtcctggacctgaaggagtacaaactcatcttcacagtgttccacatcatcgccatgtgctccacttttgccaatccccttctctatggctggatgaacagcaactacagaaaggctttcctctcggccttccgctgtgagcagcggttggatgccattcactctgaggtgtccgtgacattcaaggctaaaaagaacctggaggtcagaaagaacagtggccccaatgactctttcacagaggctaccaatgtctaaggaagctgtggtgtgaaaatgtatggatgaattc

SEQ ID NO: 38 - protein, 381 aa, Homo Sapien
MGPIGAEADENQTVEEMKVEQYGPQTTPRGELVPDPEPELIDSTKLIEVQVVLILAYCSIILLGVIGNSLVIHVVIKFKSMRTVTNFFIANLAVADLLVNTLCLPFTLTYTLMGEWKMGPVLCHLVPYAQGLAVQVSTITLTVIALDRHRCIVYHLESKISKRISFLIIGLAWGISALLASPLAIFREYSLIEIIPDFEIVACTEKWPGEEKSIYGTVYSLSSLLILYVLPLGIISFSYTRIWSKLKNHVSPGAANDHYHQRRQKTTKMLVCVVVVFAVSWLPLHAFQLAVDIDSQVLDLKEYKLIFTVFHIIAMCSTFANPLLYGWMNSNYRKAFLSAFRCEQRLDAIHSEVSVTFKAKKNLEVRKNSGPNDSFTEATNV

SEQ ID NO: 39 - DNA, 2635 bp, Homo Sapien
cgatcgccacggtccttccgccctctccttcgtccgctccatgcccaagagctgcgctccggagctggggcgaggagagccatggaggaaccgggtgctcagtgcgctccaccgccgcccgcgggctccgagacctgggttcctcaagccaacttatcctctgctccctcccaaaactgcagcgccaaggactacatttaccaggactccatctccctaccctggaaagtactgctggttatgctattggcgctcatcaccttggccaccacgctctccaatgcctttgtgattgccacagtgtaccggacccggaaactgcacaccccggctaactacctgatcgcctctctggcggtcaccgacctgcttgtgtccatcctggtgatgcccatcagcaccatgtacactgtcaccggccgctggacactgggccaggtggtctgtgacttctggctgtcgtcggacatcacttgttgcactgcctccatcctgcacctctgtgtcatcgccctggaccgctactgggccatcacggacgccgtggagtactcagctaaaaggactcccaagagggcggcggtcatgatcgcgctggtgtgggtcttctccatctctatctcgctgccgcccttcttctggcgtcaggctaaggccgaagaggaggtgtcggaatgcgtggtgaacaccgaccacatcctctacacggtctactccacggtgggtgctttctacttccccaccctgctcctcatcgccctctatggccgcatctacgtagaagcccgctcccggattttgaaacagacgcccaacaggaccggcaagcgcttgacccgagcccagctgataaccgactcccccgggtccacgtcctcggtcacctctattaactcgcgggttcccgacgtgcccagcgaatccggatctcctgtgtatgtgaaccaagtcaaagtgcgagtctccgacgccctgctggaaaagaagaaactcatggccgctagggagcgcaaagccaccaagaccctagggatcattttgggagcctttattgtgtgttggctacccttcttcatcatctccctagtgatgcctatctgcaaagatgcctgctggttccacctagccatctttgacttcttcacatggctgggctatctcaactccctcatcaaccccataatctataccatgtccaatgaggactttaaacaagcattccataaactgatacgttttaagtgcacaagttgacttgccatttgcagtggggtcgcctaagcgacctttggggaccaagttgtgtctggttccacaggtaggtcgaatcttctttcgcggtttctgggtcccagcgaggctctctctcctgggcaagggcaatggatcctgagaagccagaatagtcctgagagagagctctgaaaggagaagtgttgaaactaaatgtagagcttccctgcccaggaggaggctcacttcctcccctcaagccccgggctcagcactgaccctgcggtagccaatcccaaagggggttgcaacttttaaaaattgataatggaagggaatccctgccctgctttggtatcgtggataatgcccactagaagcagtgtacttgtaattgttgtctgaagcctgtctgagacagatctacatacagcctggcagtacttgaactagacgcttaatgccctgtgtttttggggagaactttgtgttacagcttaatttaagaacagttactttggcatcattcagtcttcactttttgtctatttaaacttggttggagaaacttgtggatttggtgcttcaaaccctatgtgtggcttggatggcgcagagaaaccttgaagagttaacagcaaaattctgatgctgagatctctatttttattatacttgaaactatatgggggtgggtgggtgggaatgggagatgaggagtgttaaactgagaatcaacacctatgattgtttgttttctgcagatttacaattttgtaattcctgtttagcgattgtcaagccacaactctaacaaacaaaccattatgtgtgctagtgccaaagtctgcagactgctttattttttctcttaatttcatgtacctgtcactttacacatttaaatccccataaatgaagggtatgatgggtgactcagcccacactgctgctatatttcttactaatgcaattggtaaaaccgattagtattggaaatatactgtttcttaacaagaaaagtgtctttatttcttatccaatttagtgagatgtgaaggagactgatgacatggggatagttcttacacaattgaggaatggggtgggggcaataggaggatgtatattttgacttgtaaaaaaatcttaaagtgcatgaaacttttatctgatagtcatttgcactctccttcccatctgtgattccttgtgtgctaacatataaagaaaccaagagaactatcttccttctccagaaaccttaaaaatacagttaagggccctaaaaacgatattgaaaagaaaataaacttgtttcttttttgttgttgttgttattgaagtttgggcaggagaaaagattgctagaaaatgacatataagaactttagaaaagctt

SEQ ID NO: 40 - protein, 390 aa, Homo Sapien
MEEPGAQCAPPPPAGSETWVPQANLSSAPSQNCSAKDYIYQDSISLPWKVLLVMLLALITLATTLSNAFVIATVYRTRKLHTPANYLIASLAVTDLLVSILVMPISTMYTVTGRWTLGQVVCDFWLSSDITCCTASILHLCVIALDRYWAITDAVEYSAKRTPKRAAVMIALVWVFSISISLPPFFWRQAKAEEEVSECVVNTDHILYTVYSTVGAFYFPTLLLIALYGRIYVEARSRILKQTPNRTGKRLTRAQLITDSPGSTSSVTSINSRVPDVPSESGSPVYVNQVKVRVSDALLEKKKLMAARERKATKTLGIILGAFIVCWLPFFIISLVMPICKDACWFHLAIFDFFTWLGYLNSLINPIIYTMSNEDFKQAFHKLIRFKCTS

SEQ ID NO: 41 - DNA, 1651 bp, Homo Sapien
tcctcttccaggatatagctgtgatgacgagtcagaagacacttggtctggtatcttcccacttgatagtgctgggaggcctccaccctcttcagccagccaggctcttagggacagagtgagctgcagagtcagtacaacccaaatacacgggctgcctgcctgagccccagcactgcctgctgcccaccaacttcccaagctggaccaagggaggcttgggtaggggccaggctagcctgagtgcacccagatgcgcttctgtcagctctccctagtgcttcaaccactgctctccctgctctactttttttgctccagctcagggatgggggtgggcagggaaatcctgccaccctcacttctccccttcccatctccaggggggccatggccagtacagagtcctccctgttgagatccctaggcctcagcccaggtcctggcagcagtgaggtggagctggactgttggtttgatgaggatttcaagttcatcctgctgcctgtgagctatgcagttgtctttgtgctgggcttgggccttaacgccccaaccctatggctcttcatcttccgcctccgaccctgggatgcaacggccacctacatgttccacctggcattgtcagacaccttgtatgtcgtgtcgctgcccaccctcatctactattatgcagcccacaaccactggccctttggcactgagatctgcaagttcgtccgctttcttttctattggaacctctactgcagtgtccttttcctcacctgcatcagcgtgcaccgctacctgggcatctgccacccacttcgggcactacgctggggccgccctcgcctcgcaggccttctctgcctggcagtttggttggtcgtagccggctgcctcgtgcccaacctgttctttgtcacaaccagcaccaaagggaccaccgtcctgtgccatgacaccactcggcctgaagagtttgaccactatgtgcacttcagctcggcggtcatggggctgctctttggcgtgccctgcctggtcactcttgtttgctatggactcatggctcgtcgcctgtatcagcccttgccaggcgctgcacagtcgtcttctcgcctccgatctctccgcaccatagctgtggtgctgactgtctttgctgtctgcttcgtgcctttccacatcacccgcaccatttactacctggccaggctgttggaagctgactgccgagtactgaacattgtcaacgtggtctataaagtgactcggcccctggccagtgccaacagctgcctggatcctgtgctctacttgctcactggggacaaatatcgacgtcagctccgtcagctctgtggtggtggcaagccccagccccgcacggctgcctcttccctggcactagtgtccctgcctgaggatagcagctgcaggtgggcggccaccccccaggacagtagctgctctactcctagggcagatagattgtaacacgggaagccgggaagtgagagaaaaggggatgagtgcagggcagaggtgagggaacccaatagtgatacctggtaaggtgcttcttccctcttttcccagggctcctggagagaagccctcaccctgaggttgcatttattgatttatatcatgggtgacc

SEQ ID NO: 42 - protein, 365 aa, Homo Sapien [sequence omitted]

SEQ ID NO: 43 - DNA, 1746 bp, Homo Sapien
agaattcggcacgacggggttctggccatgaagcccacctcaggcccagaggaggcccggcggccagcctcggacatccgcgtgttcgccagcaactgctcgatgcacgggctgggccacgtcttcgggccaggcagcctgagcctgcgccgggggatgtgggcagcggccgtggtcctgtcagtggccaccttcctctaccaggtggctgagagggtgcgctactacagggagttccaccaccagactgccctggatgagcgagaaagccaccggctcatcttcccggctgtcaccctgtgcaacatcaacccactgcgccgctcgcgcctaacgcccaacgacctgcactgggctgggtctgcgctgctgggcctggatcccgcagagcacgccgccttcctgcgcgccctgggccggccccctgcaccgcccggcttcatgcccagtcccacctttgacatggcgcaactctatgcccgtgctgggcactccctggatgacatgctgctggactgtcgcttccgtggccaaccttgtgggcctgagaacttcaccacgatcttcacccggatgggaaagtgctacacatttaactctggcgctgatggggcagagctgctcaccactactaggggtggcatgggcaatgggctggacatcatgctggacgtgcagcaggaggaatatctacctgtgtggagggacaatgaggagaccccgtttgaggtggggatccgagtgcagatccacagccaggaggagccgcccatcatcgatcagctgggcttgggggtgtccccgggctaccagacctttgtttcttgccagcagcagcagctgagcttcctgccaccgccctggggcgattgcagttcagcatctctgaaccccaactatgagccagagccctctgatcccctaggctcccccagccccagccccagccctccctatacccttatggggtgtcgcctggcctgcgaaacccgctacgtggctcggaagtgcggctgccgaatggtgtacatgccaggcgacgtgccagtgtgcagcccccagcagtacaagaactgtgcccacccggccatagatgccatgcttcgcaaggactcgtgcgcctgccccaacccgtgcgccagcacgcgctacgccaaggagctctccatggtgcggatcccgagccgcgccgccgcgcgcttcctggcccggaagctcaaccgcagcgaggcctacatcgcggagaacgtgctggccctggacatcttctttgaggccctcaactatgagaccgtggagcagaagaaggcctatgagatgtcagagctgcttggtgacattgggggccagatggggctgttcatcggggccagcctgctcaccatcctcgagatcctagactacctctgtgaggtgttccgagacaaggtcctgggatatttctggaaccgacagcactcccaaaggcactccagcaccaatctgcttcaggaagggctgggcagccatcgaacccaagttccccacctcagcctgggccccagacctcccacccctccctgtgccgtcaccaagactctctccgcctcccaccgcacctgctaccttgtcacacagctctagacctgctgtctgtgtcctcggagccccgccctgacatcctggacatgcctagcctgcacgtagcttttccgtcttcaccccaaataaagtcctaatgcatcaaaaaaaaaaaaaaaaaaaaaa

SEQ ID NO: 44 - protein, 531 aa, Homo Sapien
MKPTSGPEEARRPASDIRVFASNCSMHGLGHVFGPGSLSLRRGMWAAAVVLSVATFLYQVAERVRYYREFHHQTALDERESHRLIFPAVTLCNINPLRRSRLTPNDLHWAGSALLGLDPAEHAAFLRALGRPPAPPGFMPSPTFDMAQLYARAGHSLDDMLLDCRFRGQPCGPENFTTIFTRMGKCYTFNSGADGAELLTTTRGGMGNGLDIMLDVQQEEYLPVWRDNEETPFEVGIRVQIHSQEEPPIIDQLGLGVSPGYQTFVSCQQQQLSFLPPPWGDCSSASLNPNYEPEPSDPLGSPSPSPSPPYTLMGCRLACETRYVARKCGCRMVYMPGDVPVCSPQQYKNCAHPAIDAMLRKDSCACPNPCASTRYAKELSMVRIPSRAAARFLARKLNRSEAYIAENVLALDIFFEALNYETVEQKKAYEMSELLGDIGGQMGLFIGASLLTILEILDYLCEVFRDKVLGYFWNRQHSQRHSSTNLLQEGLGSHRTQVPHLSLGPRPPTPPCAVTKTLSASHRTCYLVTQL

SEQ ID NO: 45 - DNA, 1393 bp, Homo Sapien
ccttctcttcgtgggctatctactcagttgatccctccctcgctggcttggctctgactcctgctcagacccatcacctttgccggggaatgatgtctggagaacccctgcacgtgaagacccccatccgtgacagcatggccctgtccaaaatggccggcaccagcgtctacctcaagatggacagtgcccagccctccggctccttcaagatccggggcattgggcacttctgcaagaggtgggccaagcaaggctgtgcacattttgtctgctcctcggcgggcaacgcaggcatggcggctgcatatgcggccaggcaactcggcgtccccgccaccatcgtagtgcccggcaccacacctgctctcaccattgagcgcctcaagaatgaaggtgccacatgcaaggtggtgggtgagttattggatgaagccttcgagctggccaaggccctagcgaagaacaacccgggttgggtctacattcccccctttgatgaccccctcatctgggaaggccacgcttccatcgtgaaagagctgaaggagacactgtgggaaaagccgggggccatcgcgctgtcagtgggcggcgggggcctgctgtgtggagtggtccaggggctgcaggagtgtggctggggggacgtgcctgtcatcgccatggagacttttggtgcccacagcttccacgctgccaccaccgcaggcaaacttgtctccctgcccaagatcaccagtgttgccaaggccctgggcgtgaagactgtggggtctcaggccctgaagctgtttcaggaacaccccattttctctgaagttatctcggaccaggaggctgtggccgccattgagaagttcgtggatgatgagaagatcctggtggagcccgcctggggcgcagccctggccgctgtctatagccacgtgatccagaagctccaactggaggggaatctccgaaccccgctgccatccctcgtggtcatcgtctgcgggggcagcaacatcagcctggcccagctgcgggcgctcaaggaacagctgggcatgacaaataggttgcccaagtgaggacggaccccttaccgatctgtgctctcctagcccaagagacccctggaggggctggagtttatccagcgcctcgtcgtatgtttggctgagcacctgtggccctgggtgcaggttaacttcttgttatcaggagcccactatgcagaggccaaaggtcggcagccagcgaggctatgaattggacctttttggtatctgtgtgactgctctgtgcccatccttagccaacttgctggcgtgacaagtgcccacaagtaacacaccaggtacccagagcagggtggacaggagagacctgaatcacagcagtgagg

SEQ ID NO: 46 - protein, 328 aa, Homo Sapien
MMSGEPLHVKTPIRDSMALSKMAGTSVYLKMDSAQPSGSFKIRGIGHFCKRWAKQGCAHFVCSSAGNAGMAAAYAARQLGVPATIVVPGTTPALTIERLKNEGATCKVVGELLDEAFELAKALAKNNPGWVYIPPFDDPLIWEGHASIVKELKETLWEKPGAIALSVGGGGLLCGVVQGLQECGWGDVPVIAMETFGAHSFHAATTAGKLVSLPKITSVAKALGVKTVGSQALKLFQEHPIFSEVISDQEAVAAIEKFVDDEKILVEPAWGAALAAVYSHVIQKLQLEGNLRTPLPSLVVIVCGGSNISLAQLRALKEQLGMTNRLPK

SEQ ID NO: 47 - DNA, 1047 bp, Homo Sapien
ggagtcaacaccaacagctctgacctgggcagccttcctgagaaaatgcagccattcctcctcctgttggcctttcttctgacccctggggctgggacagaggagatcatcgggggccatgaggccaagccccactcccgcccctacatggcctttgttcagtttctgcaagagaagagtcggaagaggtgtggcggcatcctagtgagaaaggactttgtgctgacagctgctcactgccagggaagctccataaatgtcaccttgggggcccacaatatcaaggaacaggagcggacccagcagtttatccctgtgaaaagacccatcccccatccagcctataatcctaagaacttctccaacgacatcatgctactgcagctggagagaaaggccaagtggaccacagctgtgcggcctctcaggctacctagcagcaaggcccaggtgaagccagggcagctgtgcagtgtggctggctggggttatgtctcaatgagcactttagcaaccacactgcaggaagtgttgctgacagtgcagaaggactgccagtgtgaacgtctcttccatggcaattacagcagagccactgagatttgtgtgggggatccaaagaagacacagaccggtttcaagggggactccggggggcccctcgtgtgtaaggacgtagcccaaggtattctctcctatggaaacaaaaaagggacacctccaggagtctacatcaaggtctcacacttcctgccctggataaagagaacaatgaagcgcctctaacagcaggcatgagactaaccttcctctgggcctgaccatctctgggacagaggcaagaatccccaaggggtgggcagtcagggttgcaggactgtaataaatggatctctggtgtagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 48 - protein, 246 aa, Homo Sapien
MQPFLLLLAFLLTPGAGTEEIIGGHEAKPHSRPYMAFVQFLQEKSRKRCGGILVRKDFVLTAAHCQGSSINVTLGAHNIKEQERTQQFIPVKRPIPHPAYNPKNFSNDIMLLQLERKAKWTTAVRPLRLPSSKAQVKPGQLCSVAGWGYVSMSTLATTLQEVLLTVQKDCQCERLFHGNYSRATEICVGDPKKTQTGFKGDSGGPLVCKDVAQGILSYGNKKGTPPGVYIKVSHFLPWIKRTMKRL

SEQ ID NO: 49 - DNA, 884 bp, Homo Sapien
cagccacaatgaggaactcctatagatttctggcatcctctctctcagttgtcgtttctctcctgctaattcctgaagatgtctgtgaaaaaattattggaggaaatgaagtaactcctcattcaagaccctacatggtcctacttagtcttgacagaaaaaccatctgtgctggggctttgattgcaaaagactgggtgttgactgcagctcactgtaacttgaacaaaaggtcccaggtcattcttggggctcactcaataaccagggaagagccaacaaaacagataatgcttgttaagaaagagtttccctatccatgctatgacccagccacacgcgaaggtgaccttaaacttttacagctgacggaaaaagcaaaaattaacaaatatgtgactatccttcatctacctaaaaagggggatgatgtgaaaccaggaaccatgtgccaagttgcagggtggggcaggactcacaatagtgcatcttggtccgatactctgagagaagtcaatatcaccatcatagacagaaaagtctgcaatgatcgaaatcactataattttaaccctgtgattggaatgaatatggtttgtgctggaagcctccgaggtggaagagactcgtgcaatggagattctggaagccctttgttgtgcgagggtgttttccgaggggtcacttcctttggccttgaaaataaatgcggagaccctcgtgggcctggtgtctatattcttctctcaaagaaacacctcaactggataattatgactatcaagggagcagtttaaataaccgtttcctttcatttactgtggcttcttaatcttttcacaaataaaatcaatttgcatgactgtaaaaaaaaaaaaaaaaaa

SEQ ID NO: 50 - protein, 262 aa, Homo Sapien
MRNSYRFLASSLSVVVSLLLIPEDVCEKIIGGNEVTPHSRPYMVLLSLDRKTICAGALIAKDWVLTAAHCNLNKRSQVILGAHSITREEPTKQIMLVKKEFPYPCYDPATREGDLKLLQLTEKAKINKYVTILHLPKKGDDVKPGTMCQVAGWGRTHNSASWSDTLREVNITIIDRKVCNDRNHYNFNPVIGMNMVCAGSLRGGRDSCNGDSGSPLLCEGVFRGVTSFGLENKCGDPRGPGVYILLSKKHLNWIIMTIKGAV

SEQ ID NO: 51 - DNA, 1454 bp, Homo Sapien
accagcggcagaccacaggcagggcagaggcacgtctgggtcccctccctccttcctatcggcgactcccagatcctggccatgagagctccgcacctccacctctccgccgcctctggcgcccgggctctggcgaagctgctgccgctgctgatggcgcaactctgggccgcagaggcggcgctgctcccccaaaacgacacgcgcttggaccccgaagcctatggcgccccgtgcgcgcgcggctcgcagccctggcaggtctcgctcttcaacggcctctcgttccactgcgcgggtgtcctggtggaccagagttgggtgctgacggccgcgcactgcggaaacaagccactgtgggctcgagtaggggatgatcacctgctgcttcttcagggcgagcagctccgccggacgactcgctctgttgtccatcccaagtaccaccagggctcaggccccatcctgccaaggcgaacggatgagcacgatctcatgttgctaaagctggccaggcccgtagtgccggggccccgcgtccgggccctgcagcttccctaccgctgtgctcagcccggagaccagtgccaggttgctggctggggcaccacggccgcccggagagtgaagtacaacaagggcctgacctgctccagcatcactatcctgagccctaaagagtgtgaggtcttctaccctggcgtggtcaccaacaacatgatatgtgctggactggaccggggccaggacccttgccagagtgactctggaggccccctggtctgtgacgagaccctccaaggcatcctctcgtggggtgtttacccctgtggctctgcccagcatccagctgtctacacccagatctgcaaatacatgtcctggatcaataaagtcatacgctccaactgatccagatgctacgctccagctgatccagatgttatgctcctgctgatccagatgcccagaggctccatcgtccatcctcttcctccccagtcggctgaactctccccttgtctgcactgttcaaacctctgccgccctccacacctctaaacatctcccctctcacctcattcccccacctatccccattctctgcctgtactgaagctgaaatgcaggaagtggtggcaaaggtttattccagagaagccaggaagccggtcatcacccagcctctgagagcagttactggggtcacccaacctgacttcctctgccactccccgctgtgtgactttgggcaagccaagtgccctctctgaacctcagtttcctcatctgcaaaatgggaacaatgacgtgcctacctcttagacatgttgtgaggagactatgatataacatgtgtatgtaaatcttcatgtgattgtcatgtaaggcttaacacagtgggtggtgagttctgactaaaggttacctgttgtcgtgaaaaaaaaaaaaaa

SEQ ID NO: 52 - protein, 276 aa, Homo Sapien
MRAPHLHLSAASGARALAKLLPLLMAQLWAAEAALLPQNDTRLDPEAYGAPCARGSQPWQVSLFNGLSFHCAGVLVDQSWVLTAAHCGNKPLWARVGDDHLLLLQGEQLRRTTRSVVHPKYHQGSGPILPRRTDEHDLMLLKLARPVVPGPRVRALQLPYRCAQPGDQCQVAGWGTTAARRVKYNKGLTCSSITILSPKECEVFYPGVVTNNMICAGLDRGQDPCQSDSGGPLVCDETLQGILSWGVYPCGSAQHPAVYTQICKYMSWINKVIRSN

(19) United States
(12) Patent Application Publication
(10) Pub. No.: US 2003/0216288 A1
(43) Pub. Date: Nov. 20, 2003

(54) Methods and compositions for treating aids and HIV-related disorders using 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784, or 2045 molecules

(75) Inventors: Douglas M. Powell, Littleton, MA (US); Nadine S. Weich, Brookline, MA (US)

Correspondence Address: Paul J. Paglierani, Millennium Pharmaceuticals, Inc., 75 Sidney Street, Cambridge, MA, 02139, US

(73) Assignee: Millennium Pharmaceuticals, Inc.

(21) Appl. No.: 10/366,288

(22) Filed: Feb. 13, 2003

Related U.S. Application Data
(60) Provisional application No. 60/357,391, filed on Feb. 15, 2002.
(60) Provisional application No. 60/380,249, filed on May 13, 2002.
(60) Provisional application No. 60/391,306, filed on Jun. 25, 2002.
(60) Provisional application No. 60/406,297, filed on Aug. 27, 2002.
(60) Provisional application No. 60/412,007, filed on Sep. 19, 2002.
(60) Provisional application No. 60/417,508, filed on Oct. 10, 2002.
(60) Provisional application No. 60/432,318, filed on Dec. 10, 2002.

(51) Int. Cl. A61K 31/00; A61K 38/16; A61K 48/00; A61K 39/395; C12Q 1/70

(52) U.S. Cl. 514/2; 514/44; 514/1; 424/143.1; 435/5

(57) ABSTRACT

The present invention relates to methods for the diagnosis and treatment of AIDS or an HIV-related disorder or disorders. Specifically, the present invention identifies the differential expression of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 and 2045 genes in tissues relating to AIDS or an HIV-related disorder, relative to their expression in normal, or non-AIDS or HIV-related disease states, and/or in response to manipulations relevant to AIDS or an HIV-related disorder. The present invention describes methods for the diagnostic evaluation and prognosis of various HIV-related disorders, and for the identification of subjects exhibiting a predisposition to such conditions. The invention also provides methods for identifying a compound capable of modulating AIDS or an HIV-related disorder or disorders. The present invention also provides methods for the identification and therapeutic use of compounds as treatments of AIDS or an HIV-related disorder.

### RELATED APPLICATIONS

The present application claims the benefit of U.S. Provisional Application serial No. 60/357,391, filed on Feb. 15, 2002, of U.S. Provisional Application serial No. 60/380,249, filed on May 13, 2002, of U.S. Provisional Application serial No. 60/391,306, filed on Jun. 25, 2002, of U.S. Provisional Application serial No. 60/406,297, filed on Aug. 27, 2002, of U.S. Provisional Application serial No. 60/412,007, filed on Sep. 19, 2002, of U.S. Provisional Application serial No. 60/417,508, filed on Oct. 10, 2002, and of U.S. Provisional Application serial No. 60/432,318 filed on Dec. 10, 2002. The entire contents of these provisional patent applications are hereby incorporated by this reference.

### BACKGROUND OF THE INVENTION

Human Immunodeficiency Virus (HIV) is a member the lentivirus genus of the Retroviridae family. On the basis of serologic properties and sequence analysis of molecularly cloned genomes human lentivirus isolates are designated HIV-1 and HIV-2. A classification scheme based on the sequence of the viral envelope (env) protein recognizes several subtypes/clades (e.g. HIV-1 A-I). Viral diversification is a key feature of HIV phylogeny. Each subtyp displays a high degree of variability. Mutations introduced by the error-prone viral reverse transcriptase represent the major factor for variation, but also recombination occurs within individuals infected with different clades. Molecular epidemiology studies indicate, that viral migration/trafficking rather than viral mutation is the ecologic driving force for the pattern of global variation and distribution.

HIV represents an enveloped virus with two identical copies of a (+)-stranded RNA genome of 9.2 kb in length coding for 9 structural and regulatory viral proteins. Initial steps of infection are mediated through specific interaction of the viral envelope glycoprotein and the major host cell receptor CD4 as well as specific coreceptors CXCR4 (T-troph)/CCR5 (M-troph). After penetration virion RNA is converted into double-stranded DNA by the viral reverse transcriptase. Concomitantly, viral integrase and host cell proteins carry out integration of the linear DNA into the host cell genome to produce the provirus. This intracellular genomic form represents the template for synthesis of full length genomic or subgenomic (spliced and unspliced forms) single-stranded viral RNAs catalyzed by the cellular RNA polymerase II.

HIV encodes precursor polyproteins as well as additional open reading frames. The gag, pol and env genes encode precursors for the virion capsid proteins, several virion enzymes (protease, reverse transcriptase/RNAse H, integrase) as well as the envelope glycoprotein, respectively. The transcriptional activator (tat) and regulator of viral transcription (rev) encode nonstructural essential proteins. In contrast vif, vpr (HIV-1), vpu (HIV-2) and nef encoded genes represent nonessential ‘accessory’ proteins, which are thought to exert their pleiotrophic regulatory/modulatory effects through specific interactions with several different host cell encoded proteins.

Based on an intimate host/virus relationship at each step the viral life cycle is susceptible to inhibiting host cell functions. A summary of examples (see section 4.2) will illustrate the mutual relation. With the exception of the lentiviruses productive infection of target cells by most retroviruses is dependent upon proliferation and concomitant nuclear membrane dissolution of the infected cell. Lentiviruses such as HIV can infect nonproliferating cell types such as macrophages and other terminally differentiated cells overcoming the need for cell division. Activated and resting CD4-positive T helper cells as well as macrophages represent the major target cells for HIV. The role of dendritic cells as well as glia cells in HIV propagation and (neuro)-pathogenesis is discussed controversially.

HIV has been shown to be the etiologic agent of the acquired immunodeficiency syndrome (AIDS). The virus is transmitted by exposure to body fluids of an infected person. Sexual transmission, blood transfusions as well as intravenous drug abuse comprise the major routes. Infection with HIV is characterized by relentless and progressive decline in both number and function of CD4-positive T helper lymphocytes, which play a central role in coordinating immune responses. Ultimately, the weakended immune system is unable to control and eradicate the virus, AIDS develops, which is often accompanied with other opportunistic infections. In the four decades that HIV has afflicted the human population virus spread led to the death of over 22 Million people. It is estimated that about 36 million people worldwide are infected with HIV.

Antiretroviral drug therapy mainly encompassing different combinations of nucleosidic, non-nucleosidic inhibitors of the viral reverse transcriptase as well as protease inhibitors has dramatically improved the lives of those who receive drug treatment. However, current therapies only delay progression of illness and are unable to eradicate the virus. Moreover, drug resistance reappears as a significant problem, close to 50% of the patients fail to efficiently suppress viral replication on treatment mainly due to resistance issues and tolerability/compliance of current drug regimens. Thus, additional HIV therapies are urgently required.

### DETAILED DESCRIPTION OF THE INVENTION

The present invention provides methods and compositions for the diagnosis and treatment of AIDS and HIV-related disorders.

“Treatment”, as used herein, is defined as the application or administration of a therapeutic agent to a patient, or application or administration of a therapeutic agent to an isolated tissue or cell line from a patient, who has a disease or disorder, a symptom of disease or disorder or a predisposition toward a disease or disorder, with the purpose of curing, healing, alleviating, relieving, altering, remedying, ameliorating, improving or affecting the disease or disorder, at least one symptom of disease or disorder or the predisposition toward a disease or disorder. A therapeutic agent includes, but is not limited to, small molecules, peptides, antibodies, ribozymes and antisense oligonucleotides. Representative molecules are described herein.

The present invention is based, at least in part, on the discovery that nucleic acid and protein molecules, (described infra), are differentially expressed in disease states relative to their expression in normal, or non-disease states. The modulators of the molecules of the present invention, identified according to the methods of the invention can be used to modulate (e.g., inhibit, treat, or prevent) or diagnose a disease, including, but not limited to, AIDS and HIV-related disorders.

“Differential expression”, as used herein, includes both quantitative as well as qualitative differences in the temporal and/or tissue expression pattern of a gene. Thus, a differentially expressed gene may have its expression activated or inactivated in normal versus disease conditions. The degree to which expression differs in normal versus disease or control versus experimental states need only be large enough to be visualized via standard characterization techniques, e.g., quantitative PCR, Northern analysis, subtractive hybridization. The expression pattern of a differentially expressed gene may be used as part of a prognostic or diagnostic a disease, e.g., AIDS and HIV-related disorders, evaluation, or may be used in methods for identifying compounds useful for the treatment of a disease, e.g., AIDS and HIV-related disorders. In addition, a differentially expressed gene involved in a disease may represent a target gene such that modulation of the level of target gene expression or of target gene product activity will act to cure, heal, alleviate, relieve, alter, remedy, ameliorate, improve or affect a disease condition, e.g., AIDS and HIV-related disorders. Compounds that modulate target gene expression or activity of the target gene product can be used in the treatment of a disease. Although the genes described herein may be differentially expressed with respect to a disease, and/or their products may interact with gene products important to a disease, the genes may also be involved in mechanisms important to additional disease cell processes.

Molecules of the Present Invention

Gene ID 1414

The human 1414 sequence, known also as ephrin type-A receptor 2 precursor, is approximately 3921 nucleotides long including untranslated regions (SEQ ID NO:1). The coding sequence, located at about nucleic acid 114 to 3044 of SEQ ID NO:1, encodes a 976 amino acid protein (SEQ ID NO:2).

As assessed by TaqMan® analysis, 1414 mRNA was upregulated in T-cells infected with HIV. Additional TaqMan® analyses indicated that 1414 mRNA was expressed in human tissues which contain a large number of endothelial cells, e.g. human skin, intestine, lung and ovary. 1414 mRNA expression was induced in stimulated CD3 positive and CD4 positive T-cells.

1414 protein associates with src-like kinases and results in cell activation, growth and differentiation. Due to 1414 mRNA expression in T-cells infected with HIV along with its functional role, modulators of 1414 activity are useful in treating AIDS and HIV-related disorders. 1414 polypeptides of the present invention are useful to screen for modulators of 1414 activity.

Gene ID 1481

The human 1481 sequence, known also as T-cell specific kinase (Emt/Itk/Tsk), is approximately 4366 nucleotides long including untranslated regions (SEQ ID NO:3). The coding sequence, located at about nucleic acid 83 to 1945 of SEQ ID NO:3, encodes a 620 amino acid protein (SEQ ID NO:4).

As assessed by TaqMan® analysis, 1481 mRNA was expressed at very high levels in thymocytes, T-cells and macrophages. Further TaqMan® analysis indicated that 1481 mRNA was upregulated following T cell activation, in HIV infected primary CD4+ T lymphocytes.

Due to 1481 mRNA expression in thymocytes, T-cells and macrophages, along with its functional role, modulators of 1481 activity are useful in treating AIDS and HIV-related disorders. 1481 polypeptides of the present invention are useful to screen for modulators of 1481 activity.

Gene ID 1553

The human 1553 sequence, known also as microtubule affinity regulating kinase (MARK3), is approximately 3967 nucleotides long including untranslated regions (SEQ ID NO:5). The coding sequence, located at about nucleic acid 1504 to 3834 of SEQ ID NO:5, encodes a 776 amino acid protein (SEQ ID NO:6).

As assessed by TaqMan® analysis, 1553 mRNA was expressed in human adrenal gland, B-cells, brain, breast, heart, lymphocyte, osteoblast, spinal cord, T-cells, testis, thymus and thyroid. 1553 mRNA was induced in stimulated T-cells and in T-cells infected with HIV.

1553 protein is involved in the regulation of cell cycle progression. HIV infection has been shown to cause cell cycle arrest. Due to its role in vivo, and that 1553 mRNA was induced in T-cells infected with HIV, modulators of 1553 activtiy would be useful therapeutics in treating AIDS and HIV-related disorders. 1553 polypeptides of the present invention would be useful to screen for modulators of 1553 activity.

Gene ID 34021

The human 34021 sequence, also known as a serine/threonine kinase (FKSG81), is approximately 1559 nucleotides long including untranslated regions (SEQ ID NO:7). The coding sequence, located at about nucleic acid 85 to 1188 of SEQ ID NO:7, encodes a 367 amino acid protein (SEQ ID NO:8).

As assessed by TaqMan® analysis, 34021 mRNA was expressed at high levels in the two primary targets for HIV infection, T lymphocytes and macrophages. Additional TaqMan® analyses showed that 34021 mRNA expression was induced in primary T lymphocytes, T cell lines and macrophages in response to HIV infection.

T lymphocyte activation is required for viral replication. A number of kinases are involved in T cell activation following stimulation through the T cell receptor. 34021 gene expression was induced in response to T cell activation and HIV infection, suggesting that 34021 is required for viral replication. Therefore, inhibiting of the function of 34021 will inhibit T cell activation and viral replication. Due to 34021 mRNA expression in HIV-infected T-cells, along with its functional role, modulators of 34021 activity are useful in treating AIDS and HIV-related disorders. 34021 polypeptides of the present invention are useful to screen for modulators of 34021 activity

Gene ID 1720

The human 1720 sequence, known also as human tyrosine-protein kinase (ZAP-70), is approximately 3151 nucleotides long including untranslated regions (SEQ ID NO:9). The coding sequence, located at about nucleic acid 286 to 2145 of SEQ ID NO:9, encodes a 619 amino acid protein (SEQ ID NO:10).

As assessed by TaqMan® analysis, 1720 mRNA expression was restricted to T lymphoctes and T cell lines. 1720 gene expression was not induced in response to T cell activation and HIV infection, however this kinase, like many of the kinases in the T cell signaling pathway, is regulated primarily by phosphorylation, not at the level of transcription. An ORF (Open Reading Frame) analysis identified a pkinase domain as well as two SH2 domains with very high scores of 258 and 209, respectively.

T lymphocyte activation is required for viral replication. A number of kinases are involved in T cell activation following stimulation through the T cell receptor including 1720. Therefore, 1720's role in T cell activation indicates that this gene is required for viral replication. The two major mechanisms responsible for the T cell depletion seen in HIV infection are the direct cytopathic effects of viral replication in T cells, and the clearance of HIV infected cells by the immune system. Inhibition of 1720 would prevent T lymphocyte depletion by both of these mechanisms. Due to 1720 mRNA expression in T lymphoctes and T cell lines, along with its functional role, modulators of 1720 activity are useful in treating AIDS and HIV-related disorders. 1720 polypeptides of the present invention are useful to screen for modulators of 1720 activity.

Gene ID 1683

The human 1683 sequence, known also as Btk/Tec family non-receptor tyrosine kinases (TXK), is approximately 2564 nucleotides long including untranslated regions (SEQ ID NO:11). The coding sequence, located at about nucleic acid 87 to 1670 of SEQ ID NO:11, encodes a 527 amino acid protein (SEQ ID NO:12).

As assessed by TaqMan® analysis, 1683 mRNA expression was expressed at high levels in T lymphocytes, T cell lines and tissues that contain high levels of lymphocytes including tonsil and lymphnode. A recent report from Takeba et al. suggested that human TXK is expressed on Th1/Th0 cells and acts as a transcription factor that regulates gamma-IFN production.

T lymphocyte activation is required for viral replication. A number of kinases are involved in T cell activation following stimulation through the T cell receptor. The ACH2 cell line is derived from a T cell line known as CEM, containing a single integrated copy of HIV and produces high levels of virus when stimulated with tumor necrosis factor (TNF alpha). 1683 expression was induced to a higher level of expression in the unstimulated ACH2 cell line compared to the parental CEM cell line and is further induced following stimulation with TNF alpha, indicating that 1683 is required for viral replication. Therefore, the inhibition of 1683 would inhibit T cell activation and viral replication. Due to 1683 mRNA expression in T lymphoctes, T cell lines, tonsil and lymphnode, along with its functional role, modulators of 1683 activity are useful in treating AIDS and HIV-related disorders. 1683 polypeptides of the present invention are useful to screen for modulators of 1683 activity.

Gene ID 1552

The human 1552 sequence, known also as a double-stranded RNA-activated protein kinase, p68 (P1 KIN), is approximately 2562 nucleotides long including untranslated regions (SEQ ID NO:13). The coding sequence, located at about nucleic acid 187 to 1842 of SEQ ID NO:13, encodes a 551 amino acid protein (SEQ ID NO:14).

As assessed by TaqMan® analysis, 1552 mRNA expression was induced in activated T cells and T cells infected with HIV. Meurs, et al, showed that upon activation by dsRNA, in the presence of ATP, 1552 becomes autophosphorylated and can catalyze the phosphorylation of the alpha subunit of eIF2, which leads to the inhibtion of protein synthesis.

1552 enhances NFkB activation, which is essential for viral replication. This indicates that 1552 plays a role in inhibiting T cell activation and viral replication. Due to 1552 mRNA expression in T cell lines, along with its functional role, modulators of 1552 activity are useful in treating AIDS and HIV-related disorders. 1552 polypeptides of the present invention are useful to screen for modulators of 1552 activity.

Gene ID 1682

The human 1682 sequence, known also as TTK protein kinase, is approximately 3866 nucleotides long including untranslated regions (SEQ ID NO:15). The coding sequence, located at about nucleic acid 1026 to 3551 of SEQ ID NO:15, encodes a 841 amino acid protein (SEQ ID NO:16).

As assessed by TaqMan® analysis, 1682 was expressed at high levels in T lymphocytes and T cell lines. Further TaqMan® anaylsis indicated that 1682 expression was induced in HIV infected CD4+ cells.

T lymphocyte activation is required for HIV replication. A number of kinases are involved in T cell activation following stimulation through the T cell receptor. Therefore, 1682 may be required for viral replication and inhibition of 1682 would prevent T cell activation and HIV replication. Due to 1682 mRNA expression in T lymphocytes and T cell lines, along with its functional role, modulators of 1682 activity are useful in treating AIDS and HIV-related disorders. 1682 polypeptides of the present invention are useful to screen for modulators of 1682.

Gene ID 1675

The human 1675 sequence, known also as nonreceptor type protein-tyrosine kinases (TEC), is approximately 3650 nucleotides long including untranslated regions (SEQ ID NO:17). The coding sequence, located at about nucleic acid 118 to 2013 of SEQ ID NO:17, encodes a 631 amino acid protein (SEQ ID NO:18).

As assessed by TaqMan® analysis, 1675 mRNA was expressed at high levels in T lymphocytes, T cell lines and tissues that contained high levels of lymphocytes including tonsil and lymphnode. Further TaqMan® analyses, indicated that 1675 mRNA was expressed in lymphoid and myeloid cell lines. 1675 was also upregulated in SIV infected macrophages and PBMCs from rhesus macaques.

T lymphocyte activation is required for viral replication. A number of kinases are involved in T cell activation following stimulation through the T cell receptor. 1675 is known to be important in T cell activation and proliferation indicating that 1675 plays a role in T cell activation and viral replication. Due to 1675 mRNA expression in T cell lines, tonsil and lymphnode, along with its functional role, modulators of 1675 activity are useful in treating AIDS and HIV-related disorders. 1675 polypeptides of the present invention are useful to screen for modulators of 1675.

Gene ID 12825

The human 12825 sequence, known also as Ira2 Human Interleukin-1 Receptor-Associated Kinase-2 (IRAK2), is approximately 1782 nucleotides long including untranslated regions (SEQ ID NO:19). The coding sequence, located at about nucleic acid 10 to 1782 of SEQ ID NO:19, encodes a 590 amino acid protein (SEQ ID NO:20).

As assessed by TaqMan® analysis, 12825 mRNA was expressed at high levels in macrophages, which are one of two primary cell types in which HIV can replicate. 12825 was also upregulated in SIV infected PBMCs from rhesus macaques.

12825 is a Pelle family member and a MyD88 member which is a death domain-containing adapter molecule. Both molecules associate with the IL-1R signaling complex. Dominant negative forms of either molecule attenuate IL-IR-mediated NF-kB activation. Therefore, 12825 and MyD88 provides additional therapeutic targets for inhibiting IL-1-induced inflammation (Science 278:1612-1615(1997). Both IRAK and IRAK-2 are recruited to the IL-1R complex, and both appear to act upstream of TRAF6 on the pathway regulating NF-kB activation. The binding of IL-1 to the IL-1 receptor results in activation of 12825 and phosphorylation of IkB. IkB phosphorylation results in the activation of NFkB which is an essential factor for HIV transcription, indicating a role in viral replication. Inhibition of 12825 would result in decreased activation of NFkB and HIV replication. Due to 12825 mRNA expression in macrophages, along with its functional role, modulators of 12825 activity are useful in treating AIDS and HIV-related disorders. 12825 polypeptides of the present invention are useful to screen for modulators of 12825 activity.

Gene ID 9952

The human 9952 sequence, known also as a choline kinase, is approximately 2408 nucleotides long including untranslated regions (SEQ ID NO:21). The coding sequence, located at about nucleic acid 28 to 1398 of SEQ ID NO:21, encodes a 456 amino acid protein (SEQ ID NO:22).

As assessed by TaqMan® analysis, 9952 mRNA expression was upregulated in HIV infected T cell lines and primary CD4+ T cells.

9952 is a choline kinase that is involved in HIV replication through the budding of HIV from infected cell membrane called lipid rafts. These lipid rafts are enriched in phospholipids relative to other parts of the cell membrane. The membrane or envelope surrounding the HIV virion contains a higher percentage of phospholipids than the cell membrane that it comes from, therefore inhibition of 9952 results in a reduced availability of phospholipids that would interfere with viral budding and infectivity. Due to 9952 mRNA expression in T cell lines and primary CD4+ T cells, along with its functional role, modulators of 9952 activity are useful in treating AIDS and HIV-related disorders. 9952 polypeptides of the present invention are useful to screen for modulators of 9952.

Gene ID 5816

The human 5816 sequence, known also as receptor tyrosine kinase (TRKB TYROSINE KINASE), is approximately 3707 nucleotides long including untranslated regions (SEQ ID NO:23). The coding sequence, located at about nucleic acid 352 to 2820 of SEQ ID NO:23, encodes a 822 amino acid protein (SEQ ID NO:24).

As assessed by TaqMan® analysis, 5816 mRNA was upregulated in SIV infected macrophages, HIV infected primary CD4 lymphocytes, and the T cell line CEM. 5816 was also upregulated following T cell activation, indicating a role in signal transduction and or proliferation in T cells. Therefore, inhibition of 5816 results in decreased T cell activation and HIV replication. Due to 5816 mRNA expression in T cell lines, primary CD4+ T cells and macrophages, along with its functional role, modulators of 5816 activity are useful in treating AIDS and HIV-related disorders. 5816 polypeptides of the present invention are useful to screen for modulators of 5816.

Gene ID 10002

The human 10002 sequence, known also as Mitogen-Activated Protein Kinase 11 (MAP kinase p38 beta), is approximately 2180 nucleotides long including untranslated regions (SEQ ID NO:25). The coding sequence, located at about nucleic acid 20 to 1138 of SEQ ID NO:25, encodes a 372 amino acid protein (SEQ ID NO:26).

As assessed by TaqMan® analysis, 10002 mRNA expression induced T cell activation by HIV infection in the T cell line ACH2. 10002 contains all of the critical residues, including a TGY dual phosphorylation site, which, according to the primary literature, is required for its kinase activity.

T lymphocyte activation is required for viral replication. MAP kinases are involved in transmitting signals to the nucleus in response to stimulation of cell surface receptors. 10002 is known to be regulated primarily by phosphorylation, but not at the level of transcription. Because 10002 plays a role in T cell activation, it may be required for viral replication. The two major mechanisms responsible for the T cell depletion seen in HIV infection, the direct cytopathic effects of viral replication in T cells, and the clearance of HIV infected cells by the immune system. Therefore, inhibition of 10002 would prevent T lymphocyte depletion by both of these mechanisms. Due to 10002 mRNA expression in T cell lines, along with its functional role, modulators of 10002 activity are useful in treating AIDS and HIV-related disorders. 10002 polypeptides of the present invention are useful to screen for modulators of 10002.

Gene ID 1611

The human 1611 sequence, known also as Proto-Oncogene Tyrosine Protein Kinase (LCK), is approximately 2032 nucleotides long including untranslated regions (SEQ ID NO:27). The coding sequence, located at about nucleic acid 52 to 1581 of SEQ ID NO:27, encodes a 509 amino acid protein (SEQ ID NO:28).

As assessed by TaqMan® analysis, 1611 mRNA was expressed exclusively in T lymphocytes. 1611 was upregulated in HIV infected primary T cells and the T cell line ACH2, an HIV infected clone of CEM. The ACH2 cell line expressed high levels of Tat and Rev viral RNA. The high level expression of 1611 in ACH2 cells indicates that 1611 is necessary for viral production, specifically in protecting the cell from cytopathic effects of the virus. Therefore, inhibiting of 1611 would result in decreased T cell activation and viral production. Due to 1611 mRNA expression in T lymphocytes, along with its functional role, modulators of 1611 activity are useful in treating AIDS and HIV-related disorders. 1611 polypeptides of the present invention are useful to screen for modulators of 1611.

Gene ID 1371

The human 1371 sequence, known also as a tyrosine kinase (BMX), is approximately 3007 nucleotides long including untranslated regions (SEQ ID NO:29). The coding sequence, located at about nucleic acid 119 to 2212 of SEQ ID NO:29, encodes a 697 amino acid protein (SEQ ID NO:30).

As assessed by TaqMan® analysis, 1371 mRNA was expressed in lymphocytes and lymphoid tissue.

T lymphocyte activation is required for viral replication. TEC family kinases are involved in transmitting signals to the nucleus in response to stimulation of cell surface receptors. The expression of 1371 or BMX is induced following HIV infection in the T cell line H9, as well as, in macrophages and thymocytes. BMX is also regulated primarily by phosphorylation, but not at the level of transcription. Transcriptional regulation of 1371 is dramatically increased in infected thymocytes indicating that 1371 is required for viral replication. The two major mechanisms responsible for the T cell depletion in HIV infection are the direct cytopathic effects of viral replication in T cells, and the clearance of HIV infected cells by the immune system. Therefore, inhibition of 1371 prevents T lymphocyte depletion by both of these mechanisms. Due to 1371 mRNA expression in lymphocytes and lymphoid tissue, along with its functional role, modulators of 1371 activity are useful in treating AIDS and HIV-related disorders. 1371 polypeptides of the present invention are useful to screen for modulators of 1371 activity.

Gene ID 14324

The human 14324 sequence, known also as lymphocyte-expressed G-protein coupled receptor (G2A), is approximately 2588 nucleotides long including untranslated regions (SEQ ID NO:31). The coding sequence, located at about nucleic acid 901 to 2043 of SEQ ID NO:31, encodes a 380 amino acid protein (SEQ ID NO:32).

As assessed by TaqMan® analysis, 14324 mRNA was expressed in CD4+ T cells, HIV infected T cells, Tat protein treated macrophages, LPS stimulated macrophages and HIV infected thymocytes.

14324 or G2A is a GPCR involved in transmitting signals following the binding of the ligand, lysophosphatidylcholine, to the receptor. Lysophospholipids regulate different biological processes including cell proliferation and inflammation. HIV infection is characterized by chronic immune stimulation and the release of proinflammatory cytokines. In patients with HIV infection there is a correlation between the level of immune activation and disease progression. Patients with high levels of immune activation have more rapid disease progression, indicating that inhibition of the immune response to HIV results in less damage to the immune system. The expression of 14324 is induced following T cell and macrophage activation and by HIV infection of thymocytes. T lymphocyte activation is required for viral replication. The two major mechanisms responsible for the T cell depletion in HIV infection are the direct cytopathic effects of viral replication in T cells, and the clearance of HIV infected cells by the immune system. Therefore, inhibiting 14324 prevents chronic immune stimulation and T lymphocyte depletion by both of these mechanisms. Due to 14324 mRNA expression in CD4+ T cells, HIV infected T cells, Tat protein treated macrophages, LPS stimulated macrophages and HIV infected thymocytes, along with its functional role, modulators of 14324 activity are useful in treating AIDS and HIV-related disorders. 14324 polypeptides of the present invention are useful to screen for modulators of 14324 activity.

Gene ID 126

The human 126 sequence, known also as muscarinic acetylcholine receptor (M5), is approximately 2261 nucleotides long including untranslated regions (SEQ ID NO:33). The coding sequence, located at about nucleic acid 249 to 1847 of SEQ ID NO:33, encodes a 532 amino acid protein (SEQ ID NO:34).

As assessed by TaqMan® analysis, 126 mRNA was expressed at very low levels in most tissues and was expressed at higher levels in thymocytes, T-cells and T cell lines. Further TaqMan® analysis indicated that 126 mRNA was upregulated in HIV infected primary CD4+ T lymphocytes, thymocytes and in the T cell lines ACH2 and C8166.

The muscarinic acetylcholine receptor M5 is a G protein coupled receptor (GPCR). The M5 subtype is expressed at higher levels on blood mononuclear cells that in the cerebral cortex. Cells stably expressing 126 or M5 stimulate phosphatidylinositol accumulation in response to carbachol and demonstrate increased intracellular Ca++. Both of these intracellular messengers are associated with increased cell activation. Stimulation of GPCRs frequently leads to cell activation and proliferation. HIV replication requires T cell activation. The observation that this 126 or M5 is upregulated in response to HIV infection indicates a potential role for 126 or M5 in viral replication. Therefore, antagonizing 126 or M5 would provide a means to inhibit T cell activation and HIV replication. Due to 126 mRNA expression in HIV infected primary CD4+ T lymphocytes, thymocytes and in the T cell lines ACH2 and C8166, along with its functional role, modulators of 126 activity are useful in treating AIDS and HIV-related disorders. 126 polypeptides of the present invention are useful to screen for modulators of 126 activity.

Gene ID 270

The human 270 sequence, known also as prostaglandin E2 (PGE2) receptor EP2, is approximately 2372 nucleotides long including untranslated regions (SEQ ID NO:35). The coding sequence, located at about nucleic acid 157 to 1233 of SEQ ID NO:35, encodes a 358 amino acid protein (SEQ ID NO:36).

As assessed by TaqMan® analysis, 270 mRNA was expressed at very high levels in thymocytes, T-cells and macrophages. Further TaqMan® analysis indicated that 270 mRNA was upregulated following T cell activation, in HIV infected primary CD4+ T lymphocytes.

The prostaglandin E2 (PGE2) receptor is a potent immunoregulatory molecule. 270 or PGE2 induces chemotaxis in lymphocytes and monocytes. 270 or PGE2 stimulates cAMP production in cells, which results in down regulation of IL-18 induced ICAM-1 and B7.2 expression resulting in control of inflammatory and immune responses. (Takahashi, H. K., et al, J. Immunology 2002, 168:4446-4454). HIV replication requires T cell activation. Chronic immune activation in patients with HIV infection correlates with more rapid disease progression. Therefore, stable analogs of 270 or PGE2 is useful in the treatment of HIV infected individuals by decreasing T cell activation and preventing chronic immune stimulation that results in increased HIV replication and T cell depletion. Due to 270 mRNA expression in thymocytes, T-cells and macrophages, along with its functional role, modulators of 270 activity are useful in treating AIDS and HIV-related disorders. 270 polypeptides of the present invention are useful to screen for modulators of 270 activity.

Gene ID 312

The human 312 sequence, known also as hippocampal neuropeptide receptor (PYY), is approximately 1200 nucleotides long including untranslated regions (SEQ ID NO:37). The coding sequence, located at about nucleic acid 21 to 1166 of SEQ ID NO:37, encodes a 381 amino acid protein (SEQ ID NO:38).

As assessed by TaqMan® analysis, 312 mRNA was expressed at relatively low levels in most tissues and was expressed at higher levels in HIV infected cells. Further TaqMan® analysis indicated that 312 mRNA was upregulated in HIV infected primary CD4+ T lymphocytes, thymocytes and in C8166 cells.

Cells stably expressing hippocampal neuropeptide receptor (PYY) or 312 demonstrate a decrease in the accumulation of cAMP when treated with Forskolin, and stimulates the release of intracellular calcium (Gerald, C. et al, J. Biol. Chem. 1995 p26758-26761). Stimulation of GPCRs frequently leads to cell activation and proliferation, however PYY or 312 stimulates cAMP production in cells, which results in down regulation of IL-18 induced ICAM-1 and B72 expression. This results in the control of inflammatory and immune responses. (Takahashi, H. K., et al, J. Immunology 2002, 168:4446-4454). HIV replication requires T cell activation. Chronic immune activation in patients with HIV infection correlates with more rapid disease progression. Stable analogs of PGE2 is useful in the treatment of HIV infected individuals by decreasing T cell activation and preventing chronic immune stimulation that results in increased HIV replication and T cell depletion.

Transfection of PYY gene family members can cause transformation of primary fibroblasts in an agonist dependent fashion, indicating a potential role of the 312 or PYY receptors in activation and proliferation. Therefore, antagonizing 312 or PYY would provide a means to inhibit T cell activation and HIV replication. Due to 312 mRNA expression in HIV infected primary CD4+ T lymphocytes, thymocytes and in and C8166, along with its functional role, modulators of 312 activity are useful in treating AIDS and HIV-related disorders. 312 polypeptides of the present invention are useful to screen for modulators of 312 activity.

Gene ID 167

The human 167 sequence, known also as serotonin ID receptor 5-HT 1 D, a G protein coupled receptor (GPCR), is approximately 2635 nucleotides long including untranslated regions (SEQ ID NO:39). The coding sequence, located at about nucleic acid 82 to 1254 of SEQ ID NO:39, encodes a 390 amino acid protein (SEQ ID NO:40).

As assessed by TaqMan® analysis, 167 mRNA was expressed at relatively low levels in most tissues and was expressed at higher levels in HIV infected cells. 167 mRNA was upregulated in HIV infected primary CD4+ T lymphocytes, thymocytes and in the T cell lines ACH2 and C8166.

167 is the serotonin 1D receptor 5-HT 1 D, a G protein coupled receptor (GPCR). Cells stably expressing 167 or 5-HT 1D demonstrate a decrease in the accumulation of cAMP when treated with Forskolin, and does not appear to cause alterations in phosphatidylinositol metabolism. Stimulation of GPCRs frequently leads to cell activation and proliferation. Transfection of 5-HT gene family members causes transformation of primary fibroblasts in an agonist dependent fashion, indicating a role of the 5 HT receptors in activation and proliferation. HIV replication requires T cell activation. Therefore antagonizing of 5-HT 1-D would provide a means to inhibit T cell activation and HIV replication. Due to 167 mRNA expression in HIV infected primary CD4+ T lymphocytes, thymocytes and in the T cell lines ACH2 and C8166, along with its functional role, modulators of 167 activity are useful in treating AIDS and HIV-related disorders. 167 polypeptides of the present invention are useful to screen for modulators of 167 activity.

Gene ID 326

The human 326 sequence, known also as a human pyrimidinergic G protein coupled receptor (GPCR) P2Y4, is approximately 1651 nucleotides long including untranslated regions (SEQ ID NO:41). The coding sequence, located at about nucleic acid 391 to 1488 of SEQ ID NO:41, encodes a 365 amino acid protein (SEQ ID NO:42).

As assessed by TaqMan® analysis, 326 mRNA was expressed at relatively low levels in most tissues and was expressed at higher levels in T lymphocytes and macrophages. 326 mRNA was upregulated following T cell activation, in HIV infected macrophages, primary CD4+ T lymphocytes, thymocytes and in the T cell line C8166.

326 is a human pyrimidinergic G protein coupled receptor (GPCR) P2Y4 that exhibits a preference for uridine over adenine nucleotides (J. Biol. Chem. 1995. 72 No. 52: 30849-30852). Extracellular uridine nucleotides exert effects on numerous tissue and cell types. UTP and UDP are full agonists of 326, whereas ATP is a partial agonist with lower affinity that UTP. Cells expressing 326 also express inositol phosphates when stimulated with UTP or UDP. Inositol phosphates is a critical component of signal transduction. Inositol phosphates couple receptor activation with the release of calcium from calcium sequestering compartments. Stimulation of GPCRs leads to cell activation and proliferation. HIV replication requires T cell activation. Therefore antagonizing 326 will inhibit T cell activation and HIV replication. Due to 326 mRNA expression in T lymphocytes and macrophages, along with its functional role, modulators of 326 activity are useful in treating AIDS and HIV-related disorders. 326 polypeptides of the present invention are useful to screen for modulators of 326 activity.

Gene ID 18926

The human 18926 sequence, known also as an acid-sensing channel (ASIC), is approximately 1746 nucleotides long including untranslated regions (SEQ ID NO:43). The coding sequence, located at about nucleic acid 28 to 1623 of SEQ ID NO:43, encodes a 531 amino acid protein (SEQ ID NO:44).

As assessed by TaqMan® analysis, 18962 mRNA was upregulated in HIV infected primary macrophages at multiple time points. Further TaqMan® analysis indicated that 18926 mRNA was dramatically increased at the peak of infection of two T lymphocyte cell lines, H9 and C8166.

18926 is an acid-sensing channel (ASIC) that is permeable to calcium and will cause depolarization of the cell membrane. This depolarization of the call membrane results in open voltage sensitive calcium channels (VSCC's) which leads to increased accumulation of intracellular calcium. (Proc Natl Acad Sci U S A Jan. 16, 2001;98(2):711-6). Calcium is an important intracellular messenger that is released from intracellular storage compartments and the plasma membrane. Inositol triphosphate is involved in signaling through the TCR/CD3 complex resulting in T cell activation. (Cell Oct. 6, 1989;59(1):15-20). T cell activation through the TCR/CD3 complex is required for HIV replication in T lymphocytes. Therefore, antagonizing 18926 potentially inhibits signaling through the TCR/CD3 receptor resulting in decreased T cell activation and HIV replication. Cell Oct. 6, 1989;59(1):15-20. Due to 18926 mRNA expression in HIV infected primary macrophages and T lymphocyte cell lines, H9 and C8166, along with its functional role, modulators of 18926 activity are useful in treating AIDS and HIV-related disorders. 18926 polypeptides of the present invention are useful to screen for modulators of 18926 activity.

Gene ID 6747

The human 6747 sequence, known also as a serine dehydratase, is approximately 1393 nucleotides long including untranslated regions (SEQ ID NO:45). The coding sequence, located at about nucleic acid 90 to 1076 of SEQ ID NO:45, encodes a 328 amino acid protein (SEQ ID NO:46).

As assessed by TaqMan® analysis, 6747 mRNA was expressed in HIV infected T cells macrophages and thymocytes. 6747 mRNA was also expressed in C8166 cells.

6747 catalyzes the removal of ammonia from serine to generate pyruvate which serves as a source of glucose via gluconeogenisis and the synthesis of other biomolecules (J. Biol Chem Sep. 25, 1989;264(27):15818-23). T cell activation induces high levels of transcription, translation and glycosylation. All of these processes are energy dependent. 6747 expression is restricted to T cells, macrophages and liver which contain monocyte derived Kupfer cells. 6747 is induced to high levels of expression following T cell and macrophage activation and following infection with HIV. HIV infected cells are highly metabolically active, therefore inhibition of this pathway will result in decreased viral replication. Due to 6747 mRNA expression in T cells and macrophages and thymocytes, along with its functional role, modulators of 6747 activity are useful in treating AIDS and HIV-related disorders. 6747 polypeptides of the present invention are useful to screen for modulators of 6747 activity.

Gene ID 1793

The human 1793 sequence, known also as a Granzyme H, is approximately 1047 nucleotides long including untranslated regions (SEQ ID NO:47). The coding sequence, Located at about nucleic acid 46 to 786 of SEQ ID NO:47, encodes a 246 amino acid protein (SEQ ID NO:48).

As assessed by TaqMan® analysis, 1793 mRNA was found to be upregulated in HIV infected primary CD4+ T cells and HIV infected thymocytes.

1793 or Granzyme H shows the highest degree (greater than 54%) of amino acid sequence homology with granzyme B and cathepsin G. (Int Immunol January 1991;3(1):57-66). A closely related family member, cathepsin G enhances infection of macrophages with HIV. Macrophages are a major target for HIV and represent a source of infectable cells throughout the clinical course of HIV infection. Macrophages exposed to pertussis toxin prior to cathepsin G treatment, the cathepsin G-mediated effect was almost abrogated, indicating that enhancement of HIV-1 replication by cathepsin G requires Gi protein-mediated signal transduction. Cathepsin G, and other neutrophil-derived serine proteases, have multiple activities in HIV-1 infection of macrophages, including chemoattraction of monocyte/macrophages (HIV-1 targets) to inflamed tissue, activation of target cells, and increase in their susceptibility to acute HIV-1 infection. (J Virol August 2000;74(15):6849-55). Due to 1793 mRNA expression in CD4+ T cells and HIV infected T cells, along with its functional role, modulators of 1793 activity are useful in treating AIDS and HIV-related disorders. 1793 polypeptides of the present invention are useful to screen for modulators of 1793 activity.

Gene ID 1784

The human 1784 sequence, known also as Granzyme A, is approximately 884 nucleotides long including untranslated regions (SEQ ID NO:49). The coding sequence, located at about nucleic acid 9 to 797 of SEQ ID NO:49, encodes a 262 amino acid protein (SEQ ID NO:50).

As assessed by TaqMan® analysis, 1784 mRNA was expressed was in HIV infected thymocytes and primary CD4+ T cells as well as in a Jurkat T cell clone highly permissive to infection. Further TaqMan® analysis indicated that 1784 mRNA expression was highly restricted to T cells and lymphoid tissue and is further induced upon T cell activation and HIV infection.

1784 or Granzyme A is a T cell- and natural killer cell-specific trypsin-like serine RT protease that is released from effector cells during cytotoxic cell killing. (Proc Natl Acad Sci U S A February 1988;85(4):1184-8). 1784 or Granzyme A is found in the blood of normal individuals and at increased levels in patients with RA and acute EBV and HIV infection suggesting that granzymes have additional biological effects. 1784 or Granzyme A is known to induce IL-6 and IL-8 production in fibroblasts and stimulates IL-6, IL-8 and TNF alpha from monocytes. (J. I., 1998, 160: 3610-3616) Proinflamatory cytokines contribute to increased levels of immune activation and viral replication, therefore inhibition of 1784 or Granzyme A should inhibit HIV replication. Due to 1784 mRNA expression in HIV infected thymocytes and primary CD4+ T cells and Jurkat T cell, along with its functional role, modulators of 1784 activity are useful in treating AIDS and HIV-related disorders. 1784 polypeptides of the present invention are useful to screen for modulators of 1784 activity.

Gene ID 2045

The human 2045 sequence, known also as Kallikrein 10, is approximately 1454 nucleotides long including untranslated regions (SEQ ID NO:51). The coding sequence, located at about nucleic acid 82 to 912 of SEQ ID NO:51, encodes a 276 amino acid protein (SEQ ID NO:52).

As assessed by TaqMan® analysis, 2045 mRNA was expressed in HIV infected primary CD4+ T cells and HIV infected thymocytes.

2045 or Kallikrein 10 is structurally similar to polypeptides known to regulate growth factor activity (Cancer Res Jul. 15, 1996;56(14):3371-9). 2045 or Kallikrein 10 is part of a novel enzymatic cascade pathway which is down regulated in aggressive forms of ovarian and probably other cancers (Biol Chem July-August 2002;383(7-8): 1045-57) and is induced in response to HIV infection. Kallikreins are known to be involved in inflammatory and autoimmune diseases. (Endocrine Reviews 22 (2): 184-204 Copyright © 2001) Kallikreins are involved in processing peptide growth hormones which are frequently induced in HIV infection, therefore inhibition of 2045 or Kallikrein 10 results in decreased T cell activation and inflammation which is required for viral replication. Due to 2045 mRNA expression in HIV infected primary CD4+ T cells and HIV infected thymocytes, along with its functional role, modulators of 2045 activity are useful in treating AIDS and HIV-related disorders. 2045 polypeptides of the present invention are useful to screen for modulators of 2045 activity.

Various aspects of the invention are described in further detail in the following subsections:

I. Screening Assays:

The invention provides a method (also referred to herein as a “screening assay”) for identifying modulators, i.e., candidate or test compounds or agents (e.g., peptides, peptidomimetics, small molecules (organic or inorganic) or other drugs) which bind to 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins, have a stimulatory or inhibitory effect on, for example, 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression or 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity, or have a stimulatory or inhibitory effect on, for example, the expression or activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 substrate. Compounds identified using the assays described herein may be useful for treating AIDS or an HIV-related dsorder.

These assays are designed to identify compounds that bind to a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, bind to other intracellular or extracellular proteins that interact with a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, and interfere with the interaction of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein with other intercellular or extracellular proteins. For example, in the case of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, which is a transmembrane receptor-type protein, such techniques can identify ligands for such a receptor. A 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein ligand or substrate can, for example, be used to ameliorate at least one symptom of AIDS or an HIV-related disorder. Such compounds may include, but are not limited to peptides, antibodies, or small organic or inorganic compounds. Such compounds may also include other cellular proteins.

Compounds identified via assays such as those described herein may be useful, for example, for treating AIDS or an HIV-related disorder. In instances whereby AIDS or an HIV-related disorder results from an overall lower level of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene expression and/or 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein in a cell or tissue, compounds that interact with the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein may include compounds which accentuate or amplify the activity of the bound 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. Such compounds would bring about an effective increase in the level of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein activity, thus ameliorating symptoms.

In other instances, mutations within the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene may cause aberrant types or excessive amounts of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins to be made which have a deleterious effect that leads to AIDS or an HIV-related disorder. Similarly, physiological conditions may cause an excessive increase in 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene expression leading to AIDS or an HIV-related disorder. In such cases, compounds that bind to a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein may be identified that inhibit the activity of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. Assays for testing the effectiveness of compounds identified by techniques such as those described in this section are discussed herein.

In one embodiment, the invention provides assays for screening candidate or test compounds which are substrates of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or polypeptide or biologically active portion thereof. In another embodiment, the invention provides assays for screening candidate or test compounds which bind to or modulate the activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or biologically active portion thereof is contacted with a test compound and the ability of the test compound to modulate 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity is determined. Determining the ability of the test compound to modulate 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity can be accomplished by monitoring, for example, intracellular calcium, IP₃, cAMP, or diacylglycerol concentration, the phosphorylation profile of intracellular proteins, cell proliferation and/or migration, gene expression of, for example, cell surface adhesion molecules or genes associated with AIDS or an HIV-related disorder, or the activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-regulated transcription factor. The cell can be of mammalian origin, e.g., a neural cell. In one embodiment, compounds that interact with a receptor domain can be screened for their ability to function as ligands, i.e., to bind to the receptor and modulate a signal transduction pathway. Identification of ligands, and measuring the activity of the ligand-receptor complex, leads to the identification of modulators (e.g., antagonists) of this interaction. Such modulators may be useful in the treatment of AIDS or an HIV-related disorder.

The ability of the test compound to modulate 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 binding to a substrate or to bind to 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 can also be determined. Determining the ability of the test compound to modulate 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 binding to a substrate can be accomplished, for example, by coupling the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 substrate with a radioisotope or enzymatic label such that binding of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 substrate to 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 can be determined by detecting the labeled 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 substrate in a complex. 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 could also be coupled with a radioisotope or enzymatic label to monitor the ability of a test compound to modulate 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 binding to a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 substrate in a complex. Determining the ability of the test compound to bind 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 can be accomplished, for example, by coupling the compound with a radioisotope or enzymatic label such that binding of the compound to 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 can be determined by detecting the labeled 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 compound in a complex. For example, compounds (e.g., 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 ligands or substrates) can be labeled with ¹²⁵I, ³⁵S, ¹⁴C, or ³H, either directly or indirectly, and the radioisotope detected by direct counting of radioemmission or by scintillation counting. Compounds can further be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 ligand or substrate) to interact with 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 without the labeling of any of the interactants. For example, a microphysiometer can be used to detect the interaction of a compound with 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 without the labeling of either the compound or the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 (McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045.

In another embodiment, an assay is a cell-based assay comprising contacting a cell expressing a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 target molecule (e.g., a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 substrate) with a test compound and determining the ability of the test compound to modulate (e.g., stimulate or inhibit) the activity of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 target molecule. Determining the ability of the test compound to modulate the activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 target molecule can be accomplished, for example, by determining the ability of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein to bind to or interact with the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 target molecule.

Determining the ability of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or a biologically active fragment thereof, to bind to or interact with a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 target molecule can be accomplished by one of the methods described above for determining direct binding. In a preferred embodiment, determining the ability of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein to bind to or interact with a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 target molecule can be accomplished by determining the activity of the target molecule. For example, the activity of the target molecule can be determined by detecting induction of a cellular second messenger of the target (i.e., intracellular Ca²⁺, diacylglycerol, IP₃, cAMP), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response (e.g., gene expression).

In yet another embodiment, an assay of the present invention is a cell-free assay in which a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or biologically active portion thereof, is contacted with a test compound and the ability of the test compound to bind to the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or biologically active portion thereof is determined. Preferred biologically active portions of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins to be used in assays of the present invention include fragments which participate in interactions with non-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 molecules, e.g., fragments with high surface probability scores. Binding of the test compound to the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein can be determined either directly or indirectly as described above. In a preferred embodiment, the assay includes contacting the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or biologically active portion thereof with a known compound which binds 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 to form an assay mixture, contacting the assay mixture with a test compound, and determining the ability of the test compound to interact with a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, wherein determining the ability of the test compound to interact with a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein comprises determining the ability of the test compound to preferentially bind to 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 or biologically active portion thereof as compared to the known compound. Compounds that modulate the interaction of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 with a known target protein may be useful in regulating the activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, especially a mutant 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein.

In another embodiment, the assay is a cell-free assay in which a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or biologically active portion thereof is contacted with a test compound and the ability of the test compound to modulate (e.g., stimulate or inhibit) the activity of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or biologically active portion thereof is determined. Determining the ability of the test compound to modulate the activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein can be accomplished, for example, by determining the ability of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein to bind to a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 target molecule by one of the methods described above for determining direct binding. Determining the ability of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein to bind to a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 target molecule can also be accomplished using a technology such as real-time Biomolecular Interaction Analysis (BIA) (Sjolander, S. and Urbaniczky, C: (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, determining the ability of the test compound to modulate the activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein can be accomplished by determining the ability of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein to further modulate the activity of a downstream effector of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 target molecule. For example, the activity of the effector molecule on an appropriate target can be determined or the binding of the effector to an appropriate target can be determined as previously described.

In yet another embodiment, the cell-free assay involves contacting a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or biologically active portion thereof with a known compound which binds the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein to form an assay mixture, contacting the assay mixture with a test compound, and determining the ability of the test compound to interact with the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, wherein determining the ability of the test compound to interact with the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein comprises determining the ability of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein to preferentially bind to or modulate the activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 target molecule.

In more than one embodiment of the above assay methods of the present invention, it may be desirable to immobilize either 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 or its target molecule to facilitate separation of complexed from uncomplexed forms of one or both of the proteins, as well as to accommodate automation of the assay. Binding of a test compound to a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, or interaction of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 binding or activity determined using standard techniques.

Other techniques for immobilizing proteins on matrices can also be used in the screening assays of the invention. For example, either a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 target molecule can be immobilized utilizing conjugation of biotin and streptavidin. Biotinylated 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or target molecules but which do not interfere with binding of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein to its target molecule can be derivatized to the wells of the plate, and unbound target or 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein trapped in the wells by antibody conjugation. Methods for detecting such complexes, in addition to those described above for the GST-immobilized complexes, include immunodetection of complexes using antibodies reactive with the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or target molecule, as well as enzyme-linked assays which rely on detecting an enzymatic activity associated with the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or target molecule.

In another embodiment, modulators of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression are identified in a method wherein a cell is contacted with a candidate compound and the expression of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA or protein in the cell is determined. The level of expression of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA or protein in the presence of the candidate compound is compared to the level of expression of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA or protein in the absence of the candidate compound. The candidate compound can then be identified as a modulator of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression based on this comparison. For example, when expression of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA or protein is greater (statistically significantly greater) in the presence of the candidate compound than in its absence, the candidate compound is identified as a stimulator of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA or protein expression. Alternatively, when expression of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA or protein is less (statistically significantly less) in the presence of the candidate compound than in its absence, the candidate compound is identified as an inhibitor of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA or protein expression. The level of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA or protein expression in the cells can be determined by methods described herein for detecting 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA or protein.

In yet another aspect of the invention, the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 (“1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-binding proteins” or “1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-bp”) and are involved in 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity. Such 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-binding proteins are also likely to be involved in the propagation of signals by the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins or 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 targets as, for example, downstream elements of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-mediated signaling pathway. Alternatively, such 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-binding proteins are likely to be 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 inhibitors.

The two-hybrid system is based on the modular nature of most transcription factors, which consist of separable DNA-binding and activation domains. Briefly, the assay utilizes two different DNA constructs. In one construct, the gene that codes for a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein.

In another aspect, the invention pertains to a combination of two or more of the assays described herein. For example, a modulating agent can be identified using a cell-based or a cell free assay, and the ability of the agent to modulate the activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein can be confirmed in vivo, e.g., in an animal such as an animal model for AIDS or an HIV-related disorder, as described herein.

This invention further pertains to novel agents identified by the above-described screening assays. Accordingly, it is within the scope of this invention to further use an agent identified as described herein in an appropriate animal model. For example, an agent identified as described herein (e.g., a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulating agent, an antisense 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid molecule, a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-specific antibody, or a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Any of the compounds, including but not limited to compounds such as those identified in the foregoing assay systems, may be tested for the ability to ameliorate at least one symptom of AIDS or an HIV-related disorder. Cell-based and animal model-based assays for the identification of compounds exhibiting such an ability to ameliorate at least one symptom of AIDS or an HIV-related disorder are described herein.

In addition, animal-based models of AIDS or an HIV-related disorder, such as those described herein, may be used to identify compounds capable of treating AIDS or an HIV-related disorder. Such animal models may be used as test substrates for the identification of drugs, pharmaceuticals, therapies, and interventions which may be effective in treating AIDS or an HIV-related disorder. For example, animal models may be exposed to a compound, suspected of exhibiting an ability to treat AIDS or an HIV-related disorder, at a sufficient concentration and for a time sufficient to elicit such an amelioration of at least one symptom of AIDS or an HIV-related disorder in the exposed animals. The response of the animals to the exposure may be monitored by assessing the reversal of the symptoms of AIDS or an HIV-related disorder before and after treatment.

With regard to intervention, any treatments which reverse any aspect of a viral disorder (i.e. have an effect on AIDS or an HIV-related disorder) should be considered as candidates for AIDS or an HIV-related disorder therapeutic intervention. Dosages of test agents may be determined by deriving dose-response curves.

Additionally, gene expression patterns may be utilized to assess the ability of a compound to ameliorate at least one symptom of AIDS or an HIV-related disorder. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Gene expression profiles may be generated, for example, by utilizing a differential display procedure, Northern analysis and/or RT-PCR. In one embodiment, 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene sequences may be used as probes and/or PCR primers for the generation and corroboration of such gene expression profiles.

Gene expression profiles may be characterized for known states, either AIDS or an HIV-related disorder or normal, within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of AIDS or an HIV-related disorder disease model system to more closely resemble the control system. Administration of a compound may, alternatively, cause the gene expression profile of a control system to begin to mimic AIDS or an HIV-related disorder or AIDS or an HIV-related disease state. Such a compound may, for example, be used in further characterizing the compound of interest, or may be used in the generation of additional animal models.

II. Cell- and Animal-Based Model Systems

Described herein are cell- and animal-based systems which act as models for AIDS or an HIV-related disorder. These systems may be used in a variety of applications. For example, the cell- and animal-based model systems may be used to further characterize differentially expressed genes associated with AIDS or an HIV-related disorder, e.g., 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045. In addition, animal- and cell-based assays may be used as part of screening strategies designed to identify compounds which are capable of ameliorating at least one symptom of AIDS or an HIV-related disorder, as described, below. Thus, the animal- and cell-based models may be used to identify drugs, pharmaceuticals, therapies and interventions which may be effective in treating AIDS or an HIV-related disorder. Furthermore, such animal models may be used to determine the LD50 and the ED50 in animal subjects, and such data can be used to determine the in vivo efficacy of potential AIDS or HIV-related disorder treatments.

A. Animal-Based Systems

Animal-based model systems of AIDS or an HIV-related disorder may include, but are not limited to, non-recombinant and engineered transgenic animals.

Non-recombinant animal models for AIDS or an HIV-related disorder may include, for example, genetic models.

Additionally, animal models exhibiting AIDS or an HIV-related disorder may be engineered by using, for example, 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene sequences described above, in conjunction with techniques for producing transgenic animals that are well known to those of skill in the art. For example, 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene sequences may be introduced into, and overexpressed in, the genome of the animal of interest, or, if endogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene sequences are present, they may either be overexpressed or, alternatively, be disrupted in order to underexpress or inactivate 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene expression.

The host cells of the invention can also be used to produce non-human transgenic animals. For example, in one embodiment, a host cell of the invention is a fertilized oocyte or an embryonic stem cell into which 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-coding sequences have been introduced. Such host cells can then be used to create non-human transgenic animals in which exogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 sequences have been introduced into their genome or homologous recombinant animals in which endogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 sequences have been altered. Such animals are useful for studying the function and/or activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 and for identifying and/or evaluating modulators of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene has been altered by homologous recombination between the endogenous gene and an exogenous DNA molecule introduced into a cell of the animal, e.g., an embryonic cell of the animal, prior to development of the animal.

A transgenic animal used in the methods of the invention can be created by introducing a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-encoding nucleic acid into the male pronuclei of a fertilized oocyte, e.g., by microinjection, retroviral infection, and allowing the oocyte to develop in a pseudopregnant female foster animal. The 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 cDNA sequence can be introduced as a transgene into the genome of a non-human animal. Alternatively, a nonhuman homologue of a human 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene, such as a mouse or rat 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene, can be used as a transgene. Alternatively, a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene homologue, such as another 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 family member, can be isolated based on hybridization to the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 cDNA sequences and used as a transgene. Intronic sequences and polyadenylation signals can also be included in the transgene to increase the efficiency of expression of the transgene. A tissue-specific regulatory sequence(s) can be operably linked to a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 transgene to direct expression of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 transgene in its genome and/or expression of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA in tissues or cells of the animals. A transgenic founder animal can then be used to breed additional animals carrying the transgene. Moreover, transgenic animals carrying a transgene encoding a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein can further be bred to other transgenic animals carrying other transgenes.

To create a homologous recombinant animal, a vector is prepared which contains at least a portion of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene into which a deletion, addition or substitution has been introduced to thereby alter, e.g., functionally disrupt, the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene. The 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene can be a human gene but more preferably, is a non-human homologue of a human 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene. For example, a rat 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene can be used to construct a homologous recombination nucleic acid molecule, e.g., a vector, suitable for altering an endogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene in the mouse genome. In a preferred embodiment, the homologous recombination of nucleic acid molecule is designed such that, upon homologous recombination, the endogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene is functionally disrupted (i.e., no longer encodes a functional protein; also referred to as a “knock out” vector). Alternatively, the homologous recombination nucleic acid molecule can be designed such that, upon homologous recombination, the endogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene is mutated or otherwise altered but still encodes functional protein (e.g., the upstream regulatory region can be altered to thereby alter the expression of the endogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein). In the homologous recombination nucleic acid molecule, the altered portion of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene is flanked at its 5′ and 3′ ends by additional nucleic acid sequence of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene to allow for homologous recombination to occur between the exogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene carried by the homologous recombination nucleic acid molecule and an endogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene in a cell, e.g., an embryonic stem cell. The additional flanking 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene has homologously recombined with the endogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals for use in the methods of the invention can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

The 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 transgenic animals that express 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA or a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 peptide (detected immunocytochemically, using antibodies directed against 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 epitopes) at easily detectable levels should then be further evaluated to identify those animals which display a characteristic HIV-related disorder.

B. Cell-Based Systems

Cells that contain and express 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene sequences which encode a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, and, further, exhibit cellular phenotypes associated AIDS or an HIV-related disorder, may be used to identify compounds that exhibit an effect on AIDS or an HIV-related disorder. Such cells may include non-recombinant monocyte cell lines, such as U937 (ATCC# CRL-1593), THP-1 (ATCC#TIB-202), and P388D1 (ATCC# TIB-63); endothelial cells such as human umbilical vein endothelial cells (HUVECs), human microvascular endothelial cells (HMVEC), and bovine aortic endothelial cells (BAECs); as well as generic mammalian cell lines such as HeLa cells and COS cells, e.g., COS-7 (ATCC# CRL-1651), and T-cell or monocyte cell lines. Further, such cells may include recombinant, transgenic cell lines. For example, the AIDS or HIV-related disorder animal models of the invention, discussed above, may be used to generate cell lines, containing one or more cell types involved in AIDS or an HIV-related disorder, that can be used as cell culture models for this disorder. While primary cultures derived from the AIDS or HIV-related disorder model transgenic animals of the invention may be utilized, the generation of continuous cell lines is preferred. For examples of techniques which may be used to derive a continuous cell line from the transgenic animals, see Small et al., (1985) Mol. Cell Biol. 5:642-648.

Alternatively, cells of a cell type known to be involved in AIDS or an HIV-related disorder may be transfected with sequences capable of increasing or decreasing the amount of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene expression within the cell. For example, 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene sequences may be introduced into, and overexpressed in, the genome of the cell of interest, or, if endogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene sequences are present, they may be either overexpressed or, alternatively disrupted in order to underexpress or inactivate 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene expression.

In order to overexpress a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene, the coding portion of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene may be ligated to a regulatory sequence which is capable of driving gene expression in the cell type of interest, e.g., an endothelial cell. Such regulatory regions will be well known to those of skill in the art, and may be utilized in the absence of undue experimentation. Recombinant methods for expressing target genes are described above.

For underexpression of an endogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene sequence, such a sequence may be isolated and engineered such that when reintroduced into the genome of the cell type of interest, the endogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 alleles will be inactivated. Preferably, the engineered 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 sequence is introduced via gene targeting such that the endogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 sequence is disrupted upon integration of the engineered 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 sequence into the cell's genome. Transfection of host cells with 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 genes is discussed, above.

Cells treated with compounds or transfected with 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 genes can be examined for phenotypes associated with AIDS or an HIV-related disorder.

Transfection of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid may be accomplished by using standard techniques (described in, for example, Ausubel (1989) supra). Transfected cells should be evaluated for the presence of the recombinant 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene sequences, for expression and accumulation of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA, and for the presence of recombinant 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein production. In instances wherein a decrease in 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene expression is desired, standard techniques may be used to demonstrate whether a decrease in endogenous 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene expression and/or in 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein production is achieved.

III. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein and/or nucleic acid expression as well as 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity, in the context of a biological sample (e.g., blood, serum, cells, e.g., endothelial cells, or tissue, e.g., vascular tissue, lymphoid tissue, peripheral blood cells) to thereby determine whether an individual is afflicted with a predisposition or is experiencing AIDS or an HIV-related disorder. The invention also provides for prognostic (or predictive) assays for determining whether an individual is at risk of developing AIDS or an HIV-related disorder. For example, mutations in a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene can be assayed for in a biological sample. Such assays can be used for prognostic or predictive purpose to thereby phophylactically treat an individual prior to the onset of AIDS or an HIV-related disorder.

Another aspect of the invention pertains to monitoring the influence of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulators (e.g., anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibodies or 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 ribozymes) on the expression or activity of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 in clinical trials.

These and other agents are described in further detail in the following sections.

A. Diagnostic Assays

To determine whether a subject is afflicted with a disease, a biological sample may be obtained from a subject and the biological sample may be contacted with a compound or an agent capable of detecting a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or nucleic acid (e.g., mRNA or genomic DNA) that encodes a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, in the biological sample. A preferred agent for detecting 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA or genomic DNA is a labeled nucleic acid probe capable of hybridizing to 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA or genomic DNA. The nucleic acid probe can be, for example, the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid set forth in SEQ ID NO:1, 3, 5, 7, 9, 11, 13, 15, 17, 19, 21, 23, 25, 27, 29, 31, 33, 35, 37, 39, 41, 43, 45, 47, 49 or 51 or a portion thereof, such as an oligonucleotide of at least 15, 20, 25, 30, 25, 40, 45, 50, 100, 250 or 500 nucleotides in length and sufficient to specifically hybridize under stringent conditions to 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA or genomic DNA. Other suitable probes for use in the diagnostic assays of the invention are described herein.

A preferred agent for detecting 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein in a sample is an antibody capable of binding to 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA, protein, or genomic DNA in a biological sample in vitro as well as in vivo. For example, in vitro techniques for detection of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA include Northern hybridizations and in situ hybridizations. In vitro techniques for detection of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein include enzyme linked immunosorbent assays (ELISAs), Western blots, immunoprecipitations and immunofluorescence. In vitro techniques for detection of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 genomic DNA include Southern hybridizations. Furthermore, in vivo techniques for detection of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein include introducing into a subject a labeled anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibody. For example, the antibody can be labeled with a radioactive marker whose presence and location in a subject can be detected by standard imaging techniques.

In another embodiment, the methods further involve obtaining a control biological sample from a control subject, contacting the control sample with a compound or agent capable of detecting 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, mRNA, or genomic DNA, such that the presence of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, mRNA or genomic DNA is detected in the biological sample, and comparing the presence of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, mRNA or genomic DNA in the control sample with the presence of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, mRNA or genomic DNA in the test sample.

B. Prognostic Assays

The present invention further pertains to methods for identifying subjects having or at risk of developing a disease associated with aberrant 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression or activity.

As used herein, the term “aberrant” includes a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression or activity which deviates from the wild type 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression or activity. Aberrant expression or activity includes increased or decreased expression or activity, as well as expression or activity which does not follow the wild type developmental pattern of expression or the subcellular pattern of expression. For example, aberrant 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression or activity is intended to include the cases in which a mutation in the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene causes the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene to be under-expressed or over-expressed and situations in which such mutations result in a non-functional 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or a protein which does not function in a wild-type fashion, e.g., a protein which does not interact with a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 substrate, or one which interacts with a non-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 substrate.

The assays described herein, such as the preceding diagnostic assays or the following assays, can be used to identify a subject having or at risk of developing a disease. A biological sample may be obtained from a subject and tested for the presence or absence of a genetic alteration. For example, such genetic alterations can be detected by ascertaining the existence of at least one of 1) a deletion of one or more nucleotides from a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene, 2) an addition of one or more nucleotides to a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene, 3) a substitution of one or more nucleotides of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene, 4) a chromosomal rearrangement of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene, 5) an alteration in the level of a messenger RNA transcript of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene, 6) aberrant modification of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene, such as of the methylation pattern of the genomic DNA, 7) the presence of a non-wild type splicing pattern of a messenger RNA transcript of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene, 8) a non-wild type level of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-protein, 9) allelic loss of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene, and 10) inappropriate post-translational modification of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-protein.

As described herein, there are a large number of assays known in the art which can be used for detecting genetic alterations in a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene. For example, a genetic alteration in a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene may be detected using a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene (see Abravaya et al. (1995) Nucleic Acids Res. 23:675-682). This method includes collecting a biological sample from a subject, isolating nucleic acid (e.g., genomic DNA, mRNA or both) from the sample, contacting the nucleic acid sample with one or more primers which specifically hybridize to a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene under conditions such that hybridization and amplification of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene from a biological sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 can be identified by hybridizing biological sample derived and control nucleic acids, e.g., DNA or RNA, to high density arrays containing hundreds or thousands of oligonucleotide probes (Cronin, M. T. et al. (1996) Human Mutation 7:244-255; Kozal, M. J. et al. (1996) Nature Medicine 2:753-759). For example, genetic mutations in 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 can be identified in two dimensional arrays containing light-generated DNA probes as described in Cronin, M. T. et al. (1996) supra. Briefly, a first hybridization array of probes can be used to scan through long stretches of DNA in a sample and control to identify base changes between the sequences by making linear arrays of sequential, overlapping probes. This step allows for the identification of point mutations. This step is followed by a second hybridization array that allows for the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene in a biological sample and detect mutations by comparing the sequence of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 in the biological sample with the corresponding wild-type (control) sequence. Examples of sequencing reactions include those based on techniques developed by Maxam and Gilbert (1977) Proc. Natl. Acad. Sci. USA 74:560) or Sanger (1977) Proc. Natl. Acad. Sci. USA 74:5463). It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl Acad Sci USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 sequence, e.g., a wild-type 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 genes. For example, single strand conformation polymorphism (SSCP) may be used to detect differences in electrophoretic mobility between mutant and wild type nucleic acids (Orita et al. (1989) Proc Natl. Acad. Sci USA: 86:2766; see also Cotton (1993) Mutat. Res. 285:125-144 and Hayashi (1992) Genet. Anal. Tech. Appl. 9:73-79). Single-stranded DNA fragments of sample and control 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6: 1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Furthermore, the prognostic assays described herein can be used to determine whether a subject can be administered a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator (e.g., an agonist, antagonist, peptidomimetic, protein, peptide, nucleic acid, or small molecule) to effectively treat a disease.

C. Monitoring of Effects During Clinical Trials

The present invention further provides methods for determining the effectiveness of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator (e.g., a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator identified herein) in treating a disease. For example, the effectiveness of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator in increasing 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene expression, protein levels, or in upregulating 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity, can be monitored in clinical trials of subjects exhibiting decreased 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene expression, protein levels, or downregulated 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity. Alternatively, the effectiveness of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator in decreasing 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene expression, protein levels, or in downregulating 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity, can be monitored in clinical trials of subjects exhibiting increased 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene expression, protein levels, or 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity. In such clinical trials, the expression or activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene, and preferably, other genes that have been implicated in nociception can be used as a “read out” or marker of the phenotype of a particular cell.

For example, and not by way of limitation, genes, including 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045, that are modulated in cells by treatment with an agent which modulates 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity (e.g., identified in a screening assay as described herein) can be identified. Thus, to study the effect of agents which modulate 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity on subjects suffering from AIDS or an HIV-related disorder in, for example, a clinical trial, cells can be isolated and RNA prepared and analyzed for the levels of expression of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 and other genes implicated in the HIV-related disorder. The levels of gene expression (e.g., a gene expression pattern) can be quantified by Northern blot analysis or RT-PCR, as described herein, or alternatively by measuring the amount of protein produced, by one of the methods described herein, or by measuring the levels of activity of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 or other genes. In this way, the gene expression pattern can serve as a marker, indicative of the physiological response of the cells to the agent which modulates 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity. This response state may be determined before, and at various points during treatment of the individual with the agent which modulates 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity.

In a preferred embodiment, the present invention provides a method for monitoring the effectiveness of treatment of a subject with an agent which modulates 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity (e.g., an agonist, antagonist, peptidomimetic, protein, peptide, nucleic acid, or small molecule identified by the screening assays described herein) including the steps of (i) obtaining a pre-administration sample from a subject prior to administration of the agent; (ii) detecting the level of expression of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, mRNA, or genomic DNA in the pre-administration sample; (iii) obtaining one or more post-administration samples from the subject; (iv) detecting the level of expression or activity of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, mRNA, or genomic DNA in the post-administration samples; (v) comparing the level of expression or activity of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, mRNA, or genomic DNA in the pre-administration sample with the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, mRNA, or genomic DNA in the post administration sample or samples; and (vi) altering the administration of the agent to the subject accordingly. For example, increased administration of the agent may be desirable to increase the expression or activity of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 to higher levels than detected, i.e., to increase the effectiveness of the agent. Alternatively, decreased administration of the agent may be desirable to decrease expression or activity of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 to lower levels than detected, i.e. to decrease the effectiveness of the agent. According to such an embodiment, 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression or activity may be used as an indicator of the effectiveness of an agent, even in the absence of an observable phenotypic response.

IV. Methods of Treatment:

The present invention provides for both prophylactic and therapeutic methods of treating a subject, e.g., a human, at risk of (or susceptible to) a disease. With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring an subject's prophylactic or therapeutic treatment with either the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 molecules of the present invention or 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing in a subject, a disease by administering to the subject an agent which modulates 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression or 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity. Subjects at risk for AIDS or an HIV-related disorder, e.g., can be identified by, for example, any or a combination of the diagnostic or prognostic assays described herein. Administration of a prophylactic agent can occur prior to the manifestation of symptoms characteristic of aberrant 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression or activity, such that a disease is prevented or, alternatively, delayed in its progression. Depending on the type of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 aberrancy, for example, a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 agonist or 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antagonist agent can be used for treating the subject. The appropriate agent can be determined based on screening assays described herein.

B. Therapeutic Methods

Described herein are methods and compositions whereby AIDS or an HIV-related disorder may be ameliorated. Certain virological disorders are brought about, at least in part, by an excessive level of a gene product, or by the presence of a gene product exhibiting an abnormal or excessive activity. As such, the reduction in the level and/or activity of such gene products would bring about the amelioration of at least one symptom of AIDS or an HIV-related disorder. Techniques for the reduction of gene expression levels or the activity of a protein are discussed below.

Alternatively, certain other HIV-related disorders are brought about, at least in part, by the absence or reduction of the level of gene expression, or a reduction in the level of a protein's activity. As such, an increase in the level of gene expression and/or the activity of such proteins would bring about the amelioration of at least one symptom of AIDS or an HIV-related disorder.

In some cases, the up-regulation of a gene in a disease state reflects a protective role for that gene product in responding to the disease condition. Enhancement of such a gene's expression, or the activity of the gene product, will reinforce the protective effect it exerts. Some AIDS or HIV-related disease states may result from an abnormally low level of activity of such a protective gene. In these cases also, an increase in the level of gene expression and/or the activity of such gene products would bring about the amelioration of a least one symptom of AIDS or an HIV-related disorder. Techniques for increasing target gene expression levels or target gene product activity levels are discussed herein.

Accordingly, another aspect of the invention pertains to methods of modulating 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression or activity for therapeutic purposes. Accordingly, in an exemplary embodiment, the modulatory method of the invention involves contacting a cell with a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 or agent that modulates one or more of the activities of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein activity associated with the cell (e.g., an endothelial cell, ovarian cell, T-cell or monocyte). An agent that modulates 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein activity can be an agent as described herein, such as a nucleic acid or a protein, a naturally-occurring target molecule of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein (e.g., a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 ligand or substrate), a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibody, a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 agonist or antagonist, a peptidomimetic of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 agonist or antagonist, or other small molecule. In one embodiment, the agent stimulates one or more 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activities. Examples of such stimulatory agents include active 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein and a nucleic acid molecule encoding 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 that has been introduced into the cell. In another embodiment, the agent inhibits one or more 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activities. Examples of such inhibitory agents include antisense 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid molecules, anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibodies, and 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or nucleic acid molecule. In one embodiment, the method involves administering an agent (e.g., an agent identified by a screening assay described herein), or combination of agents that modulates (e.g., upregulates or downregulates) 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression or activity. In another embodiment, the method involves administering a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or nucleic acid molecule as therapy to compensate for reduced, aberrant, or unwanted 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression or activity.

Stimulation of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity is desirable in situations in which 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 is abnormally downregulated and/or in which increased 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity is likely to have a beneficial effect. Likewise, inhibition of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity is desirable in situations in which 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 is abnormally upregulated and/or in which decreased 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity is likely to have a beneficial effect.

(i) Methods for Inhibiting Target Gene Expression, Synthesis, or Activity

As discussed above, genes involved in virological disorders may cause such disorders via an increased level of gene activity. In some cases, such up-regulation may have a causative or exacerbating effect on the disease state. A variety of techniques may be used to inhibit the expression, synthesis, or activity of such genes and/or proteins.

For example, compounds such as those identified through assays described above, which exhibit inhibitory activity, may be used in accordance with the invention to ameliorate at least one symptom of AIDS or an HIV-related disorder. Such molecules may include, but are not limited to, small organic molecules, peptides, antibodies, and the like.

For example, compounds can be administered that compete with endogenous ligand for the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. The resulting reduction in the amount of ligand-bound 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein will modulate endothelial cell physiology. Compounds that can be particularly useful for this purpose include, for example, soluble proteins or peptides, such as peptides comprising one or more of the extracellular domains, or portions and/or analogs thereof, of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, including, for example, soluble fusion proteins such as Ig-tailed fusion proteins. (For a discussion of the production of Ig-tailed fusion proteins, see, for example, U.S. Pat. No. 5,116,964). Alternatively, compounds, such as ligand analogs or antibodies, that bind to the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 receptor site, but do not activate the protein, (e.g., receptor-ligand antagonists) can be effective in inhibiting 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein activity.

Further, antisense and ribozyme molecules which inhibit expression of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene may also be used in accordance with the invention to inhibit aberrant 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene activity. Still further, triple helix molecules may be utilized in inhibiting aberrant 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene activity.

The antisense nucleic acid molecules used in the methods of the invention are typically administered to a subject or generated in situ such that they hybridize with or bind to cellular mRNA and/or genomic DNA encoding a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, an antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA transcripts to thereby inhibit translation of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA. A ribozyme having specificity for a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-encoding nucleic acid can be designed based upon the nucleotide sequence of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 cDNA disclosed herein (i.e., SEQ ID NO:1, 3, 5, 7, 9, 11, 13, 15, 17, 19, 21, 23, 25, 27, 29, 31, 33, 35, 37, 39, 41, 43, 45, 47, 49 or 51). For example, a derivative of a Tetrahymena L-19 IVS RNA can be constructed in which the nucleotide sequence of the active site is complementary to the nucleotide sequence to be cleaved in a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-encoding mRNA (see, for example, Cech et al. U.S. Pat. No. 4,987,071; and Cech et al. U.S. Pat. No. 5,116,742). Alternatively, 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA can be used to select a catalytic RNA having a specific ribonuclease activity from a pool of RNA molecules (see, for example, Bartel, D. and Szostak, J. W. (1993) Science 261:1411-1418).

1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene expression can also be inhibited by targeting nucleotide sequences complementary to the regulatory region of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 (e.g., the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 promoter and/or enhancers) to form triple helical structures that prevent transcription of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene in target cells (see, for example, Helene, C. (1991) Anticancer Drug Des. 6(6):569-84; Helene, C. et al. (1992) Ann. N.Y Acad. Sci. 660:27-36; and Maher, L. J. (1992) Bioassays 14(12):807-15).

Antibodies that are both specific for the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein and interfere with its activity may also be used to modulate or inhibit 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein function. Such antibodies may be generated using standard techniques described herein, against the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein itself or against peptides corresponding to portions of the protein. Such antibodies include but are not limited to polyclonal, monoclonal, Fab fragments, single chain antibodies, or chimeric antibodies.

In instances where the target gene protein is intracellular and whole antibodies are used, internalizing antibodies may be preferred. Lipofectin liposomes may be used to deliver the antibody or a fragment of the Fab region which binds to the target epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (described in, for example, Creighton (1983), supra; and Sambrook et al. (1989) supra). Single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (1993) Proc. Natl. Acad. Sci. USA 90:7889-7893).

In some instances, the target gene protein is extracellular, or is a transmembrane protein, such as the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. Antibodies that are specific for one or more extracellular domains of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, for example, and that interfere with its activity, are particularly useful in treating AIDS or an HIV-related disorder. Such antibodies are especially efficient because they can access the target domains directly from the bloodstream. Any of the administration techniques described below which are appropriate for peptide administration may be utilized to effectively administer inhibitory target gene antibodies to their site of action.

(ii) Methods for Restoring or Enhancing Target Gene Activity

Genes that cause AIDS or an HIV-related disorder may be underexpressed within BPH and/or UI. Alternatively, the activity of the protein products of such genes may be decreased, leading to the development of AIDS or an HIV-related disorder. Such down-regulation of gene expression or decrease of protein activity might have a causative or exacerbating effect on the disease state.

In some cases, genes that are up-regulated in the disease state might be exerting a protective effect. A variety of techniques may be used to increase the expression, synthesis, or activity of genes and/or proteins that exert a protective effect in response to AIDS or an HIV-related disorder.

Described in this section are methods whereby the level of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity may be increased to levels wherein the symptoms of the HIV-related disorder are ameliorated. The level of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity may be increased, for example, by either increasing the level of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene expression or by increasing the level of active 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein which is present.

For example, a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, at a level sufficient to ameliorate at least one symptom of AIDS or an HIV-related disorder may be administered to a patient exhibiting such symptoms. Any of the techniques discussed below may be used for such administration. One of skill in the art will readily know how to determine the concentration of effective, non-toxic doses of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, utilizing techniques such as those described below.

Additionally, RNA sequences encoding a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein may be directly administered to a patient exhibiting AIDS or an HIV-related disorder, at a concentration sufficient to produce a level of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein such that AIDS or an HIV-related disorder are ameliorated. Any of the techniques discussed below, which achieve intracellular administration of compounds, such as, for example, liposome administration, may be used for the administration of such RNA molecules. The RNA molecules may be produced, for example, by recombinant techniques such as those described herein.

Further, subjects may be treated by gene replacement therapy. One or more copies of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene, or a portion thereof, that directs the production of a normal 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein with 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 function, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene sequences into human cells.

Cells, preferably, autologous cells, containing 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expressing gene sequences may then be introduced or reintroduced into the subject at positions which allow for the amelioration of at least one symptom of AIDS or an HIV-related disorder. Such cell replacement techniques may be preferred, for example, when the gene product is a secreted, extracellular gene product.

C. Pharmaceutical Compositions

Another aspect of the invention pertains to methods for treating a subject suffering from a disease. These methods involve administering to a subject an agent which modulates 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression or activity (e.g., an agent identified by a screening assay described herein), or a combination of such agents. In another embodiment, the method involves administering to a subject a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or nucleic acid molecule as therapy to compensate for reduced, aberrant, or unwanted 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 expression or activity.

Stimulation of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity is desirable in situations in which 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 is abnormally downregulated and/or in which increased 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity is likely to have a beneficial effect. Likewise, inhibition of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity is desirable in situations in which 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 is abnormally upregulated and/or in which decreased 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity is likely to have a beneficial effect.

The agents which modulate 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity (e.g., a fragment of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or an anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity can also be prepared in the form of suppositories (e.g., with conventional suppository bases such as cocoa butter and other glycerides) or retention enemas for rectal delivery.

In one embodiment, the agents that modulate 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“1L-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

D. Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agent which modulates 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity, as well as tailoring the dosage and/or therapeutic regimen of treatment with an agent which modulates 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity.

Pharmacogenomics deals with clinically significant hereditary variations in the response to drugs due to altered drug disposition and abnormal action in affected persons. See, for example, Eichelbaum, M. et al. (1996) Clin. Exp.Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein used in the methods of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 molecule or 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator used in the methods of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from AIDS or an HIV-related disorder, with an agent which modulates 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity.

V. Recombinant Expression Vectors and Host Cells Used in the Methods of the Invention

The methods of the invention (e.g., the screening assays described herein) include the use of vectors, preferably expression vectors, containing a nucleic acid encoding a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins, mutant forms of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins, fusion proteins, and the like).

The recombinant expression vectors to be used in the methods of the invention can be designed for expression of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins in prokaryotic or eukaryotic cells. For example, 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity assays, (e.g., direct assays or competitive assays described in detail below), or to generate antibodies specific for 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins. In a preferred embodiment, a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid molecule of the invention is introduced, e.g., a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid molecule within a recombinant expression vector or a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. Accordingly, the invention further provides methods for producing a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein using the host cells of the invention. In one embodiment, the method comprises culturing the host cell of the invention (into which a recombinant expression vector encoding a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein has been introduced) in a suitable medium such that a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein is produced. In another embodiment, the method further comprises isolating a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein from the medium or the host cell.

VI. Isolated Nucleic Acid Molecules Used in the Methods of the Invention

The methods of the invention include the use of isolated nucleic acid molecules that encode 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins or biologically active portions thereof, as well as nucleic acid fragments sufficient for use as hybridization probes to identify 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-encoding nucleic acid molecules (e.g., 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA) and fragments for use as PCR primers for the amplification or mutation of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51 or a portion thereof, can be isolated using standard molecular biology techniques and the sequence information provided herein. Using all or portion of the nucleic acid sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51 asa hybridization probe, 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3, 5, 7, 9, 11, 13, 15, 17, 19, 21, 23, 25, 27, 29, 31, 33, 35, 37, 39, 41, 43, 45, 47, 49 or 51 can be isolated by the polymerase chain reaction (PCR) using synthetic oligonucleotide primers designed based upon the sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51, a complement of the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51, or a portion of any of these nucleotide sequences. A nucleic acid molecule which is complementary to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51 is one which is sufficiently complementary to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51 such that it can hybridize to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51 thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 95%, 96%, 97%, 98%, 99% or more identical to the entire length of the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51, or a portion of any of this nucleotide sequence.

Moreover, the nucleic acid molecules used in the methods of the invention can comprise only a portion of the nucleic acid sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51 for example, a fragment which can be used as a probe or primer or a fragment encoding a portion of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, e.g., a biologically active portion of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51 of an anti-sense sequence of SEQ ID NO: l, 3, 5, 7, 9, 11, 13, 15, 17, 19, 21, 23, 25, 27, 29, 31, 33, 35, 37, 39, 41, 43, 45, 47, 49 or 51, or of a naturally occurring allelic variant or mutant of SEQ ID NO:1, 3, 5, 7, 9, 11, 13, 15, 17, 19, 21, 23, 25, 27, 29, 31, 33, 35, 37, 39, 41, 43, 45, 47, 49 or 51. In one embodiment, a nucleic acid molecule used in the methods of the present invention comprises a nucleotide sequence which is greater than 100, 100-200, 200-300, 300-400, 400-500, 500-600, 600-700, 700-800, 800-900, 900-1000, 1000-1100, 1100-1200, 1200-1300, or more nucleotides in length and hybridizes under stringent hybridization conditions to a nucleic acid molecule of SEQ ID NO:1, 3, 5, 7, 9, 11, 13, 15, 17, 19, 21, 23, 25, 27, 29, 31, 33, 35, 37, 39, 41, 43, 45, 47, 49 or 51.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (IxSSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(%G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₇PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, such as by measuring a level of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-encoding nucleic acid in a sample of cells from a subject e.g., detecting 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA levels or determining whether a genomic 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene has been mutated or deleted.

The methods of the invention further encompass the use of nucleic acid molecules that differ from the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51 due to degeneracy of the genetic code and thus encode the same 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins as those encoded by the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49 or 51. In another embodiment, an isolated nucleic acid molecule included in the methods of the invention has a nucleotide sequence encoding a protein having an amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50 or 52.

The methods of the invention further include the use of allelic variants of human 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045, e.g., functional and non-functional allelic variants. Functional allelic variants are naturally occurring amino acid sequence variants of the human 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein that maintain a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity. Functional allelic variants will typically contain only conservative substitution of one or more amino acids of SEQ ID NO:2, 4, 6, 8, 10, 12, 14, 16, 18, 20, 22, 24, 26, 28, 30, 32, 34, 36, 38, 40, 42, 44, 46, 48, 50 or 52 or substitution, deletion or insertion of non-critical residues in non-critical regions of the protein.

Non-functional allelic variants are naturally occurring amino acid sequence variants of the human 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein that do not have a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity. Non-functional allelic variants will typically contain a non-conservative substitution, deletion, or insertion or premature truncation of the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50 or 52, or a substitution, insertion or deletion in critical residues or critical regions of the protein.

The methods of the present invention may further use non-human orthologues of the human 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. Orthologues of the human 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein are proteins that are isolated from non-human organisms and possess the same 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity.

The methods of the present invention further include the use of nucleic acid molecules comprising the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51, or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 (e.g., the sequence of SEQ ID NO:2, 4, 6, 8, 10, 12, 14, 16, 18, 20, 22, 24, 26, 28, 30, 32, 34, 36, 38, 40, 42, 44, 46, 48, 50 or 52) without altering the biological activity, whereas an “essential” amino acid residue is required for biological activity. For example, amino acid residues that are conserved among the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins of the present invention are not likely to be amenable to alteration.

Mutations can be introduced into SEQ ID NO:1, 3, 5, 7, 9, 11, 13, 15, 17, 19, 21, 23, 25, 27, 29, 31, 33, 35, 37, 39, 41, 43, 45, 47, 49 or 51 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein is preferably replaced with another amino acid residue from the same side chain family. Alternatively, in another embodiment, mutations can be introduced randomly along all or part of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 coding sequence, such as by saturation mutagenesis, and the resultant mutants can be screened for 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 biological activity to identify mutants that retain activity. Following mutagenesis of SEQ ID NO:1, 3, 5, 7, 9, 11, 13, 15, 17, 19, 21, 23, 25, 27, 29, 31, 33, 35, 37, 39, 41, 43, 45, 47, 49 or 51, the encoded protein can be expressed recombinantly and the activity of the protein can be determined using the assay described herein.

Another aspect of the invention pertains to the use of isolated nucleic acid molecules which are antisense to the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49 or 51. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 coding strand, or to only a portion thereof. In one embodiment, an antisense nucleic acid molecule is antisense to a “coding region” of the coding strand of a nucleotide sequence encoding a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045. The term “noncoding region” refers to 5′ and 3′ sequences which flank the coding region that are not translated into amino acids (also referred to as 5′ and 3′ untranslated regions).

Given the coding strand sequences encoding 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 disclosed herein, antisense nucleic acids of the invention can be designed according to the rules of Watson and Crick base pairing. The antisense nucleic acid molecule can be complementary to the entire coding region of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA, but more preferably is an oligonucleotide which is antisense to only a portion of the coding or noncoding region of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA. For example, the antisense oligonucleotide can be complementary to the region surrounding the translation start site of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-S-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest). Antisense nucleic acid molecules used in the methods of the invention are further described above, in section IV.

In yet another embodiment, the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. (1996) Proc. Natl. Acad. Sci. 93:14670-675.

PNAs of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe et al. (1996) supra).

In another embodiment, PNAs of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. et al. (1996) supra). The synthesis of PNA-DNA chimeras can be performed as described in Hyrup B. et al. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

VII. Isolated 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 Proteins and Anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 Antibodies Used in the Methods of the Invention

The methods of the invention include the use of isolated 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins, and biologically active portions thereof, as well as polypeptide fragments suitable for use as immunogens to raise anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibodies. In one embodiment, native 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins can be isolated from cells or tissue sources by an appropriate purification scheme using standard protein purification techniques. In another embodiment, 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins are produced by recombinant DNA techniques. Alternative to recombinant expression, a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or polypeptide can be synthesized chemically using standard peptide synthesis techniques.

As used herein, a “biologically active portion” of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein includes a fragment of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein having a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity. Biologically active portions of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein include peptides comprising amino acid sequences sufficiently identical to or derived from the amino acid sequence of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, e.g., the amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50 or 52, which include fewer amino acids than the full length 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins, and exhibit at least one activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. Typically, biologically active portions comprise a domain or motif with at least one activity of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein (e.g., the N-terminal region of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein that is believed to be involved in the regulation of apoptotic activity). A biologically active portion of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein can be a polypeptide which is, for example, 25, 50, 75, 100, 125, 150, 175, 200, 250, 300 or more amino acids in length. Biologically active portions of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein can be used as targets for developing agents which modulate a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 activity.

In a preferred embodiment, the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein used in the methods of the invention has an amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50 or 52. In other embodiments, the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein is substantially identical to SEQ ID NO:2, 4, 6, 8, 10, 12, 14, 16, 18, 20, 22, 24, 26, 28, 30, 32, 34, 36, 38, 40, 42, 44, 46, 48, 50 or 52, and retains the functional activity of the protein of SEQ ID NO:2, 4, 6, 8, 10, 12, 14, 16, 18, 20, 22, 24, 26, 28, 30, 32, 34, 36, 38, 40, 42, 44, 46, 48, 50 or 52, yet differs in amino acid sequence due to natural allelic variation or mutagenesis, as described in detail in subsection V above. Accordingly, in another embodiment, the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein used in the methods of the invention is a protein which comprises an amino acid sequence at least about 50%, 55%, 60%, 65%, 70%, 75%, 80%, 85%, 90%, 95%, 96%, 97%, 98%, 99% or more identical to SEQ ID NO:2, 4, 6, 8, 10, 12, 14, 16, 18, 20, 22, 24, 26, 28, 30, 32, 34, 36, 38, 40, 42, 44, 46, 48, 50 or 52.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50 or 52, having 500 amino acid residues, at least 75, preferably at least 150, more preferably at least 225, even more preferably at least 300, and even more preferably at least 400 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0U), using a PAM120 weight residue table, a gap length penalty of 12 and a gap penalty of 4.

The methods of the invention may also use 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 chimeric or fusion proteins. As used herein, a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 “chimeric protein” or “fusion protein” comprises a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide operatively linked to a non-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide. An “1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide” refers to a polypeptide having an amino acid sequence corresponding to a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 molecule, whereas a “non-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide” refers to a polypeptide having an amino acid sequence corresponding to a protein which is not substantially homologous to the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, e.g., a protein which is different from the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein and which is derived from the same or a different organism. Within a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 fusion protein the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide can correspond to all or a portion of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. In a preferred embodiment, a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 fusion protein comprises at least one biologically active portion of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. In another preferred embodiment, a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 fusion protein comprises at least two biologically active portions of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. Within the fusion protein, the term “operatively linked” is intended to indicate that the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide and the non-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide are fused in-frame to each other. The non-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide can be fused to the N-terminus or C-terminus of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide.

For example, in one embodiment, the fusion protein is a GST-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 fusion protein in which the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 sequences are fused to the C-terminus of the GST sequences. Such fusion proteins can facilitate the purification of recombinant 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045.

In another embodiment, this fusion protein is a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein containing a heterologous signal sequence at its N-terminus. In certain host cells (e.g., mammalian host cells), expression and/or secretion of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 can be increased through use of a heterologous signal sequence.

The 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 fusion proteins used in the methods of the invention can be incorporated into pharmaceutical compositions and administered to a subject in vivo. The 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 fusion proteins can be used to affect the bioavailability of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 substrate. Use of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 fusion proteins may be useful therapeutically for the treatment of disorders caused by, for example, (i) aberrant modification or mutation of a gene encoding a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein; (ii) mis-regulation of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 gene; and (iii) aberrant post-translational modification of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein.

Moreover, the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-fusion proteins used in the methods of the invention can be used as immunogens to produce anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibodies in a subject, to purify 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 ligands and in screening assays to identify molecules which inhibit the interaction of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 with a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 substrate.

Preferably, a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-encoding nucleic acid can be cloned into such an expression vector such that the fusion moiety is linked in-frame to the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein.

The present invention also pertains to the use of variants of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins which function as either 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 agonists (mimetics) or as 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antagonists. Variants of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins can be generated by mutagenesis, e.g., discrete point mutation or truncation of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. An agonist of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins can retain substantially the same, or a subset, of the biological activities of the naturally occurring form of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. An antagonist of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein can inhibit one or more of the activities of the naturally occurring form of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein by, for example, competitively modulating a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045-mediated activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein.

In one embodiment, variants of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein which function as either 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 agonists (mimetics) or as 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antagonists can be identified by screening combinatorial libraries of mutants, e.g., truncation mutants, of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein for 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein agonist or antagonist activity. In one embodiment, a variegated library of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 variants is generated by combinatorial mutagenesis at the nucleic acid level and is encoded by a variegated gene library. A variegated library of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 variants can be produced by, for example, enzymatically ligating a mixture of synthetic oligonucleotides into gene sequences such that a degenerate set of potential 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 sequences is expressible as individual polypeptides, or alternatively, as a set of larger fusion proteins (e.g., for phage display) containing the set of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 sequences therein. There are a variety of methods which can be used to produce libraries of potential 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein coding sequence can be used to generate a variegated population of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 fragments for screening and subsequent selection of variants of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. In one embodiment, a library of coding sequence fragments can be generated by treating a double stranded PCR fragment of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with SI nuclease, and ligating the resulting fragment library into an expression vector. By this method, an expression library can be derived which encodes N-terminal, C-terminal and internal fragments of various sizes of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 variants (Arkin and Yourvan (1992) Proc. Natl. Acad. Sci. USA 89:7811-7815; Delgrave et al. (1993) Protein Engineering 6(3):327-331).

The methods of the present invention further include the use of anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibodies. An isolated 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein, or a portion or fragment thereof, can be used as an immunogen to generate antibodies that bind 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 using standard techniques for polyclonal and monoclonal antibody preparation. A full-length 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein can be used or, alternatively, antigenic peptide fragments of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 can be used as immunogens. The antigenic peptide of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 comprises at least 8 amino acid residues of the amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50 or 52, and encompasses an epitope of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 such that an antibody raised against the peptide forms a specific immune complex with the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 that are located on the surface of the protein, e.g., hydrophilic regions, as well as regions with high antigenicity.

A 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 immunogen is typically used to prepare antibodies by immunizing a suitable subject, (e.g., rabbit, goat, mouse, or other mammal) with the immunogen. An appropriate immunogenic preparation can contain, for example, recombinantly expressed 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein or a chemically synthesized 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide. The preparation can further include an adjuvant, such as Freund's complete or incomplete adjuvant, or similar immunostimulatory agent. Immunization of a suitable subject with an immunogenic 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 preparation induces a polyclonal anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibody response.

The term “antibody” as used herein refers to immunoglobulin molecules and immunologically active portions of immunoglobulin molecules, i.e., molecules that contain an antigen binding site which specifically binds (immunoreacts with) an antigen, such as a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045. Examples of immunologically active portions of immunoglobulin molecules include F(ab) and F(ab)₂ fragments which can be generated by treating the antibody with an enzyme such as pepsin. The invention provides polyclonal and monoclonal antibodies that bind 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 molecules. The term “monoclonal antibody” or “monoclonal antibody composition”, as used herein, refers to a population of antibody molecules that contain only one species of an antigen binding site capable of immunoreacting with a particular epitope of 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045. A monoclonal antibody composition thus typically displays a single binding affinity for a particular 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein with which it immunoreacts.

Polyclonal anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibodies can be prepared as described above by immunizing a suitable subject with a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 immunogen. The anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibody titer in the immunized subject can be monitored over time by standard techniques, such as with an enzyme linked immunosorbent assay (ELISA) using immobilized 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045. If desired, the antibody molecules directed against 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 can be isolated from the mammal (e.g., from the blood) and further purified by well known techniques, such as protein A chromatography to obtain the IgG fraction. At an appropriate time after immunization, e.g., when the anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 immunogen as described above, and the culture supernatants of the resulting hybridoma cells are screened to identify a hybridoma producing a monoclonal antibody that binds 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045.

Any of the many well known protocols used for fusing lymphocytes and immortalized cell lines can be applied for the purpose of generating an anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 monoclonal antibody (see, e.g., G. Galfre et al. (1977) Nature 266:55052; Gefter et al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045, e.g., using a standard ELISA assay.

Alternative to preparing monoclonal antibody-secreting hybridomas, a monoclonal anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibody can be identified and isolated by screening a recombinant combinatorial immunoglobulin library (e.g., an antibody phage display library) with 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 to thereby isolate immunoglobulin library members that bind 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045. Kits for generating and screening phage display libraries are commercially available (e.g., the Pharmacia Recombinant Phage Antibocly System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. (1990) Nature 348:552-554.

Additionally, recombinant anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

An anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibody can be used to detect 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein (e.g., in a cellular lysate or cell supernatant) in order to evaluate the abundance and pattern of expression of the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 protein. Anti-1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

This invention is further illustrated by the following examples which should not be construed as limiting. The contents of all references, patents and published patent applications cited throughout this application, as well as the Figure and the Sequence Listing is incorporated herein by reference.

### EXAMPLES

### Example 1

### Tissue Distribution of Using TaqMan® Analysis

This example describes the TaqMan® procedure. The TaqMan® procedure is a quantitative, reverse transcription PCR-based approach for detecting mRNA. The RT-PCR reaction exploits the 5′ nuclease activity of AmpliTaq Gold™ DNA Polymerase to cleave a TaqMan® probe during PCR. Briefly, CDNA was generated from the samples of interest, e.g., heart, kidney, liver, skeletal muscle, and various vessels, and used as the starting material for PCR amplification. In addition to the 5′ and 3′ gene-specific primers, a gene-specific oligonucleotide probe (complementary to the region being amplified) was included in the reaction (i.e., the TaqMan® probe). The TaqMan® probe includes the oligonucleotide with a fluorescent reporter dye covalently linked to the 5′ end of the probe (such as FAM (6-carboxyfluorescein), TET (6-carboxy-4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method for identifying a compound capable of treating AIDS or an HIV-related disorder, comprising assaying the ability of the compound to modulate 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid expression or 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide activity, thereby identifying a compound capable of treating aids or an hiv-related disorder.

2. A method for identifying a compound associated with AIDS or an HIV-related disorder comprising:
a) contacting a cell which expresses 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 with a test compound; and
b) assaying the ability of the test compound to modulate the expression of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid, or the activity of a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide, thereby identifying a compound associated with AIDS or an HIV-related disorder.

3. A method for modulating AIDS or an HIV-related disorder in a cell comprising contacting a cell with a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator, thereby modulating AIDS or an HIV-related disorder in the cell.

4. The method of claim 2, wherein the cell is a T cell.

5. The method of claim 3, wherein the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator is a small organic molecule, peptide, antibody or antisense nucleic acid molecule.

6. The method of claim 3, wherein the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator is capable of modulating 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide activity.

7. The method of claim 6, wherein the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator is a small organic molecule, peptide, antibody or antisense nucleic acid molecule.

8. The method of claim 6, wherein the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator is capable of modulating 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid expression.

9. A method for treating a subject having AIDS or an HIV-related disorder characterized by aberrant 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide activity or aberrant 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 nucleic acid expression comprising administering to the subject a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator, thereby treating said subject having AIDS or an HIV-related disorder.

10. The method of claim 9, wherein said 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator is administered in a pharmaceutically acceptable formulation.

11. The method of claim 9, wherein the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator is a small organic molecule, peptide, antibody or antisense nucleic acid molecule.

12. The method of claim 9, wherein the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator is capable of modulating 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide activity.

13. A method for modulating viral replication in a cell comprising contacting a cell with a 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator, thereby modulating viral replication in the cell.

14. The method of claim 13, wherein the cell is a T cell.

15. The method of claim 13, wherein the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator is a small organic molecule, peptide, antibody or antisense nucleic acid molecule.

16. The method of claim 13, wherein the 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 modulator is capable of modulating 1414, 1481, 1553, 34021, 1720, 1683, 1552, 1682, 1675, 12825, 9952, 5816, 10002, 1611, 1371, 14324, 126, 270, 312, 167, 326, 18926, 6747, 1793, 1784 or 2045 polypeptide activity.
